(19) United States
(12) Patent Application Publication
(10) Pub. No.: US 2006/0264494 A1
(43) Pub. Date: Nov. 23, 2006

(54) Heterocyclic amide derivatives which process glycogen phorylase inhibitory activity

(75) Inventors: Stuart Norman Lile Bennett, Cheshire (GB); Iain Simpson, Macclesfield (GB); Paul Robert Owen Whittamore, Macclesfield (GB)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 10/567,801

(22) PCT Filed: Aug. 18, 2004
(86) PCT No.: PCT/GB04/03546

§ 371 Date: Feb. 9, 2006

(30) Foreign Application Priority Data
Aug. 22, 2003 (GB) 0319759.7

(51) Int. Cl. A61K 31/407; C07D 498/02

(52) U.S. Cl. 514/412; 548/453

(57) ABSTRACT

A compound of the formula (1) or a pharmaceutically-acceptable salt, or pro-drug thereof; (1) wherein, for example: R⁴ and R⁵ together are either —S—C(R⁶═C(R⁷)— or —C(R⁷)═C(R⁶)—S—; R⁶ and R⁷ are independently selected from hydrogen and halo; A is phenylene or heteroarylene; n is 0, 1 or 2; R¹ is halo, cyano or carboxy; R² is for example methyl; R³ is for example selected from halo(1-4C)alkyl, dihalo(1-4C)alkyl, tifluoromethyl, hydroxy(1-4C)alkyl, dihydroxy(2-4C)alkyl, trihydroxy(3-4C)alkyl, cyano(1-4C)alkyl (optionally substituted on alkyl with hydroxy), (1-4C)alkoxy(1-4C)alkyl, (1-4C)alkoxy(1-4C)alkoxy(1-4C)alkyl, di[(1-4C)alkoxy](1-4C)alkyl, (hydroxy)[(1-4C)alkoxy](1-4C)alkyl; possess glycogen phosphorylase inhibitory activity and accordingly have value in the treatment of disease states associated with increased glycogen phosphorylase activity. Processes for the manufacture of compounds and pharmaceutical compositions containing them are described.

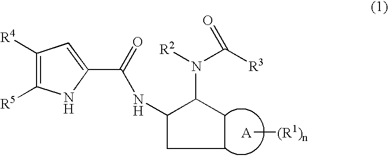

The present invention relates to heterocyclic amide derivatives, pharmaceutically acceptable salts and in-vivo hydrolysable esters thereof. These heterocyclic amides possess glycogen phosphorylase inhibitory activity and accordingly have value in the treatment of disease states associated with increased glycogen phosphorylase activity and thus are potentially useful in methods of treatment of a warm-blooded animal such as man. The invention also relates to processes for the manufacture of said heterocyclic amide derivatives, to pharmaceutical compositions containing them and to their use in the manufacture of medicaments to inhibit glycogen phosphorylase activity in a warm-blooded animal such as man.

The liver is the major organ regulating glycaemia in the post-absorptive state. Additionally, although having a smaller role in the contribution to post-prandial blood glucose levels, the response of the liver to exogenous sources of plasma glucose is key to an ability to maintain euglycaemia. An increased hepatic glucose output (HGO) is considered to play an important role in maintaining the elevated fasting plasma glucose (FPG) levels seen in type 2 diabetics; particularly those with a FPG>140 mg/dl (7.8 mM). (Weyer et al, (1999), J Clin Invest 104: 787-794; Clore & Blackgard (1994), Diabetes 43: 256-262; De Fronzo, R. A., et al, (1992) Diabetes Care 15; 318-355; Reaven, G. M. (1995) Diabetologia 38; 3-13).

Since current oral, anti-diabetic therapies fail to bring FPG levels to within the normal, non-diabetic range and since raised FPG (and glycHbA1c) levels are risk factors for both macro- (Charles, M. A. et al (1996) Lancet 348, 1657-1658; Coutinho, M. et al (1999) Diabetes Care 22; 233-240; Shaw, J. E. et al (2000) Diabetes Care 23, 34-39) and micro-vascular disease (DCCT Research Group (1993) New. Eng. J. Med. 329; 977-986); the reduction and normalisation of elevated FPG levels remains a treatment goal in type 2 DM.

It has been estimated that, after an overnight fast, 74% of HGO was derived from glycogenolysis with the remainder derived from gluconeogenic precursors (Hellerstein et al (1997) Am J Physiol, 272: E163). Glycogen phosphorylase is a key enzyme in the generation by glycogenolysis of glucose-1-phosphate, and hence glucose in liver and also in other tissues such as muscle and neuronal tissue.

Liver glycogen phosphorylase a activity is elevated in diabetic animal models including the db/db mouse and the fa/fa rat (Aiston S et al (2000). Diabetalogia 43, 589-597).

Inhibition of hepatic glycogen phosphorylase with chloroindole inhibitors (CP91149 and CP320626) has been shown to reduce both glucagon stimulated glycogenolysis and glucose output in hepatocytes (Hoover et al (1998) J Med Chem 41, 2934-8; Martin et al (1998) PNAS 95, 1776-81). Additionally, plasma glucose concentration is reduced, in a dose related manner, db/db and ob/ob mice following treatment with these compounds.

Studies in conscious dogs with glucagon challenge in the absence and presence of another glycogen phosphorylase inhibitor, Bay K 3401, also show the potential utility of such agents where there is elevated circulating levels of glucagon, as in both Type 1 and Type 2 diabetes. In the presence of Bay R 3401, hepatic glucose output and arterial plasma glucose following a glucagon challenge were reduced significantly (Shiota et al, (1997), Am J Physiol, 273: E868).

The heterocyclic amides of the present invention possess glycogen phosphorylase inhibitory activity and accordingly are expected to be of use in the treatment of type 2 diabetes, insulin resistance, syndrome X, hyperinsulinaemia, hyperglucagonaemia, cardiac ischaemia and obesity, particularly type 2 diabetes.

Our patent application WO 02/20530 discloses a spectrum of active glycogen phosphorylase inhibitors, amongst which are a very limited number of amino-indan containing compounds.

Our co-pending patent applications PCT/GB03/00883 and PCT/GB03/00875 disclose a variety of substituted amino-indan glycogen phosphorylase inhibitors, generally containing only one substitutent on the nitrogen of the amino-indan moiety, although a number are disubstituted and contain an N-acetyl group as one substituent.

Surprisingly, we have found that a group of N-disubstituted amino-indans have improved physical properties (for example solubility, plasma-protein binding) in comparison with that of the compounds previously disclosed, which are particularly beneficial for a pharmaceutical.

According to one aspect of the present invention there is provided a compound of formula (1): 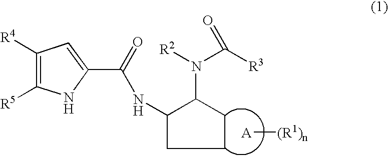
wherein:
R⁴ and R⁵ together are either —S—C(R⁶)═C(R⁷)— or —C(R⁷)═C(R⁶)—S—;
R⁶ and R⁷ are independently selected from hydrogen, halo, nitro, cyano, hydroxy, fluoromethyl, difluoromethyl, trifluoromethyl, trifluoromethoxy, carboxy, carbamoyl, (1-4C)alkyl, (2-4C)alkenyl, (2-4C)alkynyl, (1-4C)alkoxy and (1-4C)alkanoyl;
A is phenylene or heteroarylene;
n is 0, 1 or 2;
R¹ is independently selected from halo, nitro, cyano, hydroxy, carboxy, carbamoyl,
N-(1-4C)alkylcarbamoyl, N,N-((1-4C)alkyl)₂carbamoyl, sulphamoyl, N-(1-4C)alkylsulphamoyl, N,N-((1-4C)alkyl)₂sulphamoyl, —S(O)_{b}(1-4C)alkyl (wherein b is 0, 1, or 2), —OS(O)₂(1-4C)alkyl, (1-4C)alkyl, (2-4C)alkenyl, (2-4C)alkynyl, (1-4C)alkoxy, (1-4C)alkanoyl, (1-4C)alkanoyloxy, hydroxy(1-4C)alkyl, fluoromethyl, difluoromethyl, trifluoromethyl, trifluoromethoxy and —NHSO₂(1-4C)alkyl;
or, when n is 2, the two R¹ groups, together with the carbon atoms of A to which they are attached, may form a 4 to 7 membered saturated ring, optionally containing 1 or 2 heteroatoms independently selected from O, S and N, and optionally being substituted by one or two methyl groups;
one of R² and R³ is selected from R_{N}a, and the other is selected from R_{N}b;
R_{N}a: (1-3C)alkyl, halo(1-3C)alkyl, dihalo(1-3)alkyl, trifluoromethyl, hydroxy(1-3C)alkyl, dihydroxy(2-3C)alkyl, cyano(1-3C)alkyl (optionally substituted on alkyl with hydroxy), methoxymethyl, ethoxymethyl, methoxyethyl, methoxymethoxymethyl, dimethoxyethyl, (hydroxy)(methoxy)ethyl, 5- and 6-membered acetals and mono- and di-methyl derivatives thereof, (amino)(hydroxy)(2-3C)alkyl, (aminocarbonyl)(hydroxy)(2-3C)alkyl, (methylaminocarbonyl)(hydroxy)(2-3C)alkyl, (dimethylaminocarbonyl)(hydroxy)(2-3C)alkyl, (methylcarbonylamino)(hydroxy)(2-3C)alkyl, (methylS(O)ₚ-)(hydroxy)(2-3C)alkyl (wherein p is 0, 1 or 2);
R_{N}b: (1-4C)alkyl, halo(1-4C)alkyl, dihalo(1-4C)alkyl, trifluoromethyl, hydroxy(1-4C)alkyl, dihydroxy(2-4C)alkyl, trihydroxy(3-4C)alkyl, cyano(1-4C)alkyl (optionally substituted on alkyl with hydroxy), (1-4C)alkoxy(1-4C)alkyl, (1-4C)alkoxy(1-4C)alkoxy(1-4C)alkyl, di[(1-4C)alkoxy](2-4C)alkyl, (hydroxy)[(1-4C)alkoxy](2-4C)alkyl, 5- and 6-membered acetals and mono- and di-methyl derivatives thereof, (amino)(hydroxy)(2-4C)alkyl, (aminocarbonyl)(hydroxy)(2-4C)alkyl, ((1-4C)alkylaminocarbonyl)(hydroxy)(2-4C)alkyl, (di(1-4C)alkylaminocarbonyl)(hydroxy)(2-4C)alkyl, ((1-4C)alkylcarbonylamino)(hydroxy)(2-4C)alkyl, ((1-4C)alkylS(O)ₚ-)(hydroxy)(2-4C)alkyl (wherein p is 0, 1 or 2);
wherein any alkyl or alkoxy group within any group in R_{N}A and R_{N}B may also optionally be substituted on an available carbon atom with a hydroxy group (provided that said carbon atom is not already substituted by a group linked by a heteroatom);
provided that if R² is (1-3C)alkyl or (1-4C)alkyl then R³ is not (1-4C)alkyl or (1-3C)alkyl;
or a pharmaceutically acceptable salt or pro-drug thereof.

In another aspect of the invention, there is provided a compound of the formula (1) or a pharmaceutically acceptable salt or pro-drug thereof wherein one of R² and R³ is selected from R_{N}a, and the other is selected from R_{N}b; and R_{N}a and R_{N}b are selected from:
R_{N}a: (1-3C)alkyl, dihalo(1-3)alkyl, trifluoromethyl, hydroxy(1-3C)alkyl, dihydroxy(2-3C)alkyl, cyano(1-3C)alkyl, methoxymethyl, ethoxymethyl, methoxyethyl, methoxymethoxymethyl, dimethoxyethyl, (hydroxy)(methoxy)ethyl, 5- and 6-membered acetals and mono- and di-methyl derivatives thereof;
R_{N}b: (1-4C)alkyl, halo(1-4C)alkyl, dihalo(1-4C)alkyl, trifluoromethyl, hydroxy(1-4C)alkyl, dihydroxy(2-4C)alkyl, trihydroxy(3-4C)alkyl, cyano(1-4C)alkyl, (1-4C)alkoxy(1-4C)alkyl, (1-4C)alkoxy(1-4C)alkoxy(1-4C)alkyl, di[(1-4C)alkoxy](2-4C)alkyl, (hydroxy)[(1-4C)alkoxy](2-4C)alkyl, 5- and 6-membered acetals and mono- and di-methyl derivatives thereof;
and wherein R¹, R⁴, A, m and n are as hereinbefore defined.

It is to be understood that when A is heteroarylene, the bridgehead atoms joining ring A to the ring may be heteroatoms. Therefore, for example, the definition of 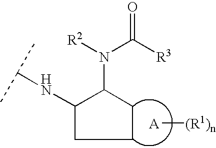
when A is heteroarylene encompasses the structures:

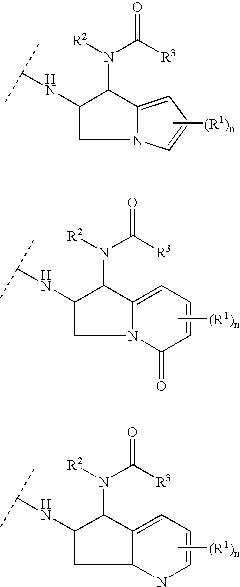

It is to be understood that where substituents contain two substituents on an alkyl chain, in which both are linked by a heteroatom (for example two alkoxy substituents, or an amino and a hydroxy substituent), then these two substituents are not substituents on the same carbon atom of the alkyl chain.

In another aspect, the invention relates to compounds of formula (1) as hereinabove defined or to a pharmaceutically acceptable salt.

In another aspect, the invention relates to compounds of formula (1) as hereinabove defined or to a pro-drug thereof. Suitable examples of pro-drugs of compounds of formula (1) are in-vivo hydrolysable esters of compounds of formula (1). Therefore in another aspect, the invention relates to compounds of formula (1) as hereinabove defined or to an in-vivo hydrolysable ester thereof.

It is to be understood that, insofar as certain of the compounds of formula (1) defined above may exist in optically active or racemic forms by virtue of one or more asymmetric carbon atoms, the invention includes in its definition any such optically active or racemic form which possesses glycogen phosphorylase inhibition activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

Within the present invention it is to be understood that a compound of the formula (1) or a salt thereof may exhibit the phenomenon of tautomerism and that the formulae drawings within this specification can represent only one of the possible tautomeric forms. It is to be understood that the invention encompasses any tautomeric form which has glycogen phosphorylase inhibition activity and is not to be limited merely to any one tautomeric form utilised within the formulae drawings. The formulae drawings within this specification can represent only one of the possible tautomeric forms and it is to be understood that the specification encompasses all possible tautomeric forms of the compounds drawn not just those forms which it has been possible to show graphically herein.

It is also to be understood that certain compounds of the formula (1) and salts thereof can exist in solvated as well as unsolvated forms such as, for example, hydrated forms. It is to be understood that the invention encompasses all such solvated forms which have glycogen phosphorylase inhibition activity.

It is also to be understood that certain compounds of the formula (1) may exhibit polymorphism, and that the invention encompasses all such forms which possess glycogen phosphorylase inhibition activity.

The present invention relates to the compounds of formula (1) as hereinbefore defined as well as to the salts thereof. Salts for use in pharmaceutical compositions will be pharmaceutically acceptable salts, but other salts may be useful in the production of the compounds of formula (1) and their pharmaceutically acceptable salts. Pharmaceutically acceptable salts of the invention may, for example, include acid addition salts of the compounds of formula (1) as hereinbefore defined which are sufficiently basic to form such salts. Such acid addition salts include for example salts with inorganic or organic acids affording pharmaceutically acceptable anions such as with hydrogen halides (especially hydrochloric or hydrobromic acid of which hydrochloric acid is particularly preferred) or with sulphuric or phosphoric acid, or with trifluoroacetic, citric or maleic acid. Suitable salts include hydrochlorides, hydrobromides, phosphates, sulphates, hydrogen sulphates, alkylsulphonates, arylsulphonates, acetates, benzoates, citrates, maleates, fumarates, succinates, lactates and tartrates. In addition where the compounds of formula (1) are sufficiently acidic, pharmaceutically acceptable salts may be formed with an inorganic or organic base which affords a pharmaceutically acceptable cation. Such salts with inorganic or organic bases include for example an alkali metal salt, such as a sodium or potassium salt, an alkaline earth metal salt such as a calcium or magnesium salt, an ammonium salt or for example a salt with methylamine, dimethylamine, trimethylamine, piperidine, morpholine or tris-(2-hydroxyethyl)amine.

The compounds of the invention may be administered in the form of a pro-drug which is broken down in the human or animal body to give a compound of the invention. A prodrug may be used to alter or improve the physical and/or pharmacokinetic profile of the parent compound and can be formed when the parent compound contains a suitable group or substituent which can be derivatised to form a prodrug. Examples of pro-drugs include in-vivo hydrolysable esters of a compound of the invention or a pharmaceutically-acceptable salt thereof.

Various forms of prodrugs are known in the art, for examples see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem Pharm Bull, 32, 692 (1984).

An in-vivo hydrolysable ester of a compound of formula (1) containing carboxy or hydroxy group is, for example, a pharmaceutically acceptable ester which is cleaved in the human or animal body to produce the parent acid or alcohol.

Suitable pharmaceutically acceptable esters for carboxy include alkyl, (1-6C)alkoxymethyl esters for example methoxymethyl, (1-6C)alkanoyloxymethyl esters for example pivaloyloxymethyl, phthalidyl esters, (3-8C)cycloalkoxycarbonyloxy(1-6C)alkyl esters for example 1-cyclohexylcarbonyloxyethyl; 1,3-dioxolen-2-onylmethyl esters for example 5-methyl-1,3-dioxolen-2-onylmethyl; and (1-6C)alkoxycarbonyloxyethyl esters for example 1-methoxycarbonyloxyethyl and may be formed at any carboxy group in the compounds of this invention.

Suitable pharmaceutically-acceptable esters for hydroxy include inorganic esters such as phosphate esters (including phosphoramidic cyclic esters) and α-acyloxyalkyl ethers and related compounds which as a result of the in-vivo hydrolysis of the ester breakdown to give the parent hydroxy groups. Examples of α-acyloxyalkyl ethers include acetoxymethoxy and 2,2-dimethylpropionyloxymethoxy. A selection of in-vivo hydrolysable ester forming groups for hydroxy include (1-10C)alkanoyl, for example acetyl; benzoyl; phenylacetyl; substituted benzoyl and phenylacetyl, (1-10C)alkoxycarbonyl (to give alkyl carbonate esters), for example ethoxycarbonyl; di-((1-4C))alkylcarbamoyl and N-(di-((1-4C))alkylaminoethyl)-N-((1-4C)) alkylcarbamoyl (to give carbamates); di-((1-4C))alkylaminoacetyl and carboxyacetyl. Examples of ring substituents on phenylacetyl and benzoyl include aminomethyl, ((1-4C))alkylaminomethyl and di-(((1-4C))alkyl)aminomethyl, and morpholino or piperazino linked from a ring nitrogen atom via a methylene linking group to the 3- or 4-position of the benzoyl ring. Other interesting in-vivo hydrolysable esters include, for example, R^{A}C(O)O((1-6C))alkyl-CO—, wherein R^{A} is for example, benzyloxy-((1-4C))alkyl, or phenyl). Suitable substituents on a phenyl group in such esters include, for example, 4-((1-4C))piperazino-((1-4C))alkyl, piperazino-((1-4C))alkyl and morpholino(1-4C)alkyl.

In this specification the generic term “alkyl” includes both straight-chain and branched-chain alkyl groups. However references to individual alkyl groups such as “propyl” are specific for the straight chain version only and references to individual branched-chain alkyl groups such as t-butyl are specific for the branched chain version only. For example, “(1-3C)alkyl” includes methyl, ethyl, propyl and isopropyl, “(1-4C)alkyl” includes methyl, ethyl, propyl, isopropyl and t-butyl and examples of “(1-6C)alkyl” include the examples of “(1-4C)alkyl” and additionally pentyl, 2,3-dimethylpropyl, 3-methylbutyl and hexyl. An analogous convention applies to other generic terms, for example “(2-4C)alkenyl” includes vinyl, allyl and 1-propenyl and examples of “(2-6C)alkenyl” include the examples of “(2-4C)alkenyl” and additionally 1-butenyl, 2-butenyl, 3-butenyl, 2-methylbut-2-enyl, 3-methylbut-1-enyl, 1-pentenyl, 3-pentenyl and 4-hexenyl. Examples of “(2-4C)alkynyl” includes ethynyl, 1-propynyl and 2-propynyl and examples of “C₂₋₆alkynyl” include the examples of “(2-4C)alkynyl” and additionally 3-butynyl, 2-pentynyl and 1-methylpent-2-ynyl.

The term “hydroxy(1-3C)alkyl” includes hydroxymethyl, hydroxyethyl, hydroxypropyl and hydroxyisopropyl. The term “hydroxy(2-3C)alkyl” includes hydroxyethyl, hydroxypropyl and hydroxyisopropyl. The term “hydroxy(1-4C)alkyl” includes hydroxymethyl, hydroxyethyl, hydroxypropyl, hydroxyisopropyl and hydroxybutyl. The term “hydroxy(1-4C)alkyl” also includes hydroxycyclopropyl and hydroxycyclobutyl. The term “hydroxyethyl” includes 1-hydroxyethyl and 2-hydroxyethyl. The term “hydroxypropyl” includes 1-hydroxypropyl, 2-hydroxypropyl and 3-hydroxypropyl and an analogous convention applies to terms such as hydroxybutyl. The term “dihydroxy(2-3C)alkyl” includes dihydroxyethyl, dihydroxypropyl and dihydroxyisopropyl. The term “dihydroxy(2-4C)alkyl” includes dihydroxyethyl, dihydroxypropyl, dihydroxyisopropyl and dihydroxybutyl. The term “dihydroxypropyl” includes 1,2-dihydroxypropyl, 2,3-dihydroxypropyl and 1,3-dihydroxypropyl. An analogous convention applies to terms such as dihydroxyisopropyl and dihydroxybutyl. The term dihydroxy(2-4C)alkyl is not intended to include structures which are geminally disubstituted and thereby unstable.

The term “trihydroxy(3-4C)alkyl” includes 1,2,3-trihydroxypropyl and 1,2,3-trihydroxybutyl. The term trihydroxy(3-4C)alkyl is not intended to include structures which are geminally di- or tri-substituted and thereby unstable.

The term “halo” refers to fluoro, chloro, bromo and iodo. The term “halo(1-3C)alkyl” includes fluoromethyl, chloromethyl, fluoroethyl, fluoropropyl and chloropropyl. The term “halo(1-4C)alkyl” includes “halo(1-3C)alkyl” and additionally fluorobutyl. The term “dihalo(1-4C)alkyl” includes difluoromethyl and dichloromethyl. The term “dihalo(1-3C)alkyl” includes difluoromethyl and dichloromethyl. The term “trihalo(1-4C)alkyl” includes trifluoromethyl.

Examples of “5- and 6-membered cyclic acetals and mono- and di-methyl derivatives thereof” are:
1,3-dioxolan-4-yl, 2-methyl-1,3-dioxolan-4-yl, 2,2-dimethyl-1,3-dioxolan-4-yl; 2,2-dimethyl-1,3-dioxan-4-yl; 2,2-dimethyl-1,3-dioxan-5-yl; 1,3-dioxan-2-yl.

Examples of “(1-4C)alkoxy” include methoxy, ethoxy, propoxy and isopropoxy. Examples of “(1-6C)alkoxy” include the examples of “(1-4C)alkoxy” and additionally butyloxy, t-butyloxy, pentoxy and 1,2-(methyl)₂propoxy. Examples of “(1-4C)alkanoyl” include formyl, acetyl and propionyl. Examples of “(1-6C)alkanoyl” include the example of “(1-4C)alkanoyl” and additionally butanoyl, pentanoyl, hexanoyl and 1,2-(methyl)₂propionyl. Examples of “(1-4C)alkanoyloxy” are formyloxy, acetoxy and propionoxy. Examples of “(1-6C)alkanoyloxy” include the examples of “(1-4C)alkanoyloxy” and additionally butanoyloxy, pentanoyloxy, hexanoyloxy and 1,2-(methyl)₂propionyloxy. Examples of “N-((1-4C)alkyl)amino” include methylamino and ethylamino. Examples of “N-((1-6C)alkyl)amino” include the examples of “N-((1-4C)alkyl)amino” and additionally pentylamino, hexylamino and 3-methylbutylamino. Examples of “N,N-((1-4C)alkyl)₂amino” include N-N-(methyl)₂amino, N-N-(ethyl)₂amino and N-ethyl-N-methylamino. Examples of “N,N-((1-6C)alkyl)₂amino” include the example of “N,N-((1-4C)alkyl)₂amino” and additionally N-methyl-N-pentylamino and N,N-(pentyl)₂amino. Examples of “N-((1-4C)alkyl)carbamoyl” are methylcarbamoyl and ethylcarbamoyl. Examples of “N-((1-6C)alkyl)carbamoyl” are the examples of “N-((1-4C)alkyl)carbamoyl” and additionally pentylcarbamoyl, hexylcarbamoyl and 1,2-(methyl)₂propylcarbamoyl. Examples of “N,N-((1-4C)alkyl)₂carbamoyl” are N,N-(methyl)₂carbamoyl, N,N-(ethyl)₂carbamoyl and N-methyl-N-ethylcarbamoyl. Examples of “N,N-((1-6C)alkyl)₂carbamoyl” are the examples of “N,N-((1-4C)alkyl)₂carbamoyl” and additionally N,N-(pentyl)₂carbamoyl, N-methyl-N-pentylcarbamoyl and N-ethyl-N-hexylcarbamoyl. Examples of “N-((1-4C)alkyl)sulphamoyl” are N-(methyl)sulphamoyl and N-(ethyl)sulphamoyl. Examples of “N-((1-6C)alkyl)sulphamoyl” are the examples of “N-((1-4C)alkyl)sulphamoyl” and additionally N-pentylsulphamoyl, N-hexylsulphamoyl and 1,2-(methyl)₂propylsulphamoyl. Examples of “N,N-((1-4C)alkyl)₂sulphamoyl” are N,N-(methyl)₂sulphamoyl, N,N-(ethyl)₂sulphamoyl and N-(methyl)-N-(ethyl)sulphamoyl. Examples of “N,N-((1-6C)alkyl)₂sulphamoyl” are the examples of “N,N-((1-4C)alkyl)₂sulphamoyl” and additionally N,N-(pentyl)₂sulphamoyl, N-methyl-N-pentylsulphamoyl and N-ethyl-N-hexylsulphamoyl.

Examples of “cyano(1-3C)alkyl” and “cyano(1-4C)alkyl” are cyanomethyl, cyanoethyl and cyanopropyl. Examples of “(3-6C)cycloalkyl” include cyclopropyl, cyclobutyl, cyclopentyl and cyclohexyl. Examples of “(3-6C)cycloalkyl(1-4C)alkyl” include cyclopropylmethyl, cyclopropylethyl, cyclopropylpropyl, cyclobutylmethyl, cyclopentylmethyl and cyclohexylmethyl. Examples of “cyano(1-3C)alkyl” and “cyano(1-4C)alkyl” substituted with hydroxy include 1-(hydroxy)-2-(cyano)ethyl.

The term “amino(1-4C)alkyl” includes aminomethyl, aminoethyl, aminopropyl, aminoisopropyl and aminobutyl. The term “aminoethyl” includes 1-aminoethyl and 2-aminoethyl. The term “aminopropyl” includes 1-aminopropyl, 2-aminopropyl and 3-aminopropyl and an analogous convention applies to terms such as aminoethyl and aminobutyl.

Examples of “(1-4C)alkoxy(1-4C)alkoxy” are methoxymethoxy, ethoxymethoxy, ethoxyethoxy and methoxyethoxy. Examples of “hydroxy(1-4C)alkoxy” are hydroxyethoxy and hydroxypropoxy. Examples of “hydroxypropoxy” are 2-hydroxypropoxy and 3-hydroxypropoxy. Examples of “(1-4C)alkoxy(1-4C)alkyl” include methoxymethyl, ethoxymethyl, methoxyethyl, ethoxypropyl and propoxymethyl. Examples of “(1-4C)alkoxy(1-4C)alkoxy(1-4C)alkyl” include methoxymethoxymethyl, ethoxyethoxyethyl, ethoxymethoxymethyl, methoxyethoxymethyl, methoxymethoxyethyl, methoxyethoxyethyl and ethoxymethoxymethyl. Examples of “di[(1-4C)alkoxy](2-4C)alkyl” include 1,2-dimethoxyethyl, 2,3-dimethoxypropyl and 1-methoxy-2-ethoxy-ethyl. Examples of “(hydroxy)[(1-4C)alkoxy](2-4C)alkyl” include 1-hydroxy-2-methoxyethyl and 1-hydroxy-3-methoxypropyl.

Examples of “—S(O)_{b}(1-4C)alkyl (wherein b is 0, 1 or 2)” include methylthio, ethylthio, propylthio, methylsulphinyl, ethylsulphinyl, propanesulphinyl, mesyl, ethylsulphonyl, propylsulphony and isopropylsulphonyl.

Examples of “(1-6C)alkoxycarbonyl” include methoxycarbonyl, ethoxycarbonyl, n-and t-butoxycarbonyl.

Examples of “(amino)(hydroxy)(2-3C)alkyl” and “(amino)(hydroxy)(2-4C)alkyl” include 1-amino-2-hydroxyethyl, 1-hydroxy-2-aminoethyl, 1-hydroxy-2-aminopropyl and 1-amino-2-hydroxypropyl. Examples of “(aminocarbonyl)(hydroxy)(2-3C)alkyl” and “(aminocarbonyl)(hydroxy)(2-4C)alkyl” include 1-(hydroxy)-2-(aminocarbonyl)ethyl and 1-(hydroxy)-3-(aminocarbonyl)propyl. Examples of “((1-4C)alkylaminocarbonyl)(hydroxy)(2-4C)alkyl” and “(methylaminocarbonyl)(hydroxy)(2-3C)alkyl” include 1-(hydroxy)-2-(N-methylaminocarbonyl)ethyl. Examples of “(di(1-4C)alkylaminocarbonyl)(hydroxy)(2-4C)alkyl” and “(dimethylaminocarbonyl)(hydroxy)(2-3C)alkyl” include 1-(hydroxy)-2-(N,N-dimethylaminocarbonyl)ethyl. Examples of “(1-4C)alkylcarbonylamino)(hydroxy)(2-4C)alkyl” and “methylcarbonylamino)(hydroxy)(2-3C)alkyl” include 1-hydroxy-2-(methylcarbonylamino)ethyl and 1-(methylcarbonylamino)-2-(hydroxy)ethyl.

Examples of “((1-4C)alkylS(O)p-)(hydroxy)(2-4C)alkyl” and “(methylS(O)p-)(hydroxy)(2-4C)alkyl” (wherein p is 0, 1 or 2) include 1-(hydroxy)-2-(methylthio)ethyl, 1-(hydroxy)-2-(methylsulfinyl)ethyl and 1-(hydroxy)-2-(methylsulfonyl)ethyl.

Examples of additional substitution on an alkyl or alkoxy group within a definition of R_{N}A and R_{N}B by hydroxy is to be understood to mean, for example, substitution of a hydroxy in di(halo)(1-4C)alkyl to give groups such as 1-hydroxy-2,2-difluoromethyl; or for example substitution of a hydroxy into an (amino)(hydroxy)(2-4C)alkyl group to give a group such as 1,2-dihydroxy-3-aminopropyl; or for example substitution of a hydroxy into a “((1-4C)alkylS(O)p-)(hydroxy)(2-4C)alkyl,” to give for example HOCH₂CH₂S(O)₂CH₂CH(OH)—, or C₂H₅S(O)₂CH₂CH(OH)CH(OH)—.

Within this specification composite terms are used to describe groups comprising more that one functionality such as -(1-4C)alkylSO₂(1-4C)alkyl. Such terms are to be interpreted in accordance with the meaning which is understood by a person skilled in the art for each component part. For example -(1-4C)alkylSO₂(1-4C)alkyl includes -methylsulphonylmethyl, -methylsulphonylethyl, -ethylsulphonylmethyl, and -propylsulphonylbutyl.

Where optional substituents are chosen from “0, 1, 2 or 3” groups it is to be understood that this definition includes all substituents being chosen from one of the specified groups or the substituents being chosen from two or more of the specified groups. An analogous convention applies to substituents chose from “0, 1 or 2” groups and “1 or 2” groups.

“Heteroarylene” is a diradical of a heteroaryl group. A heteroaryl group is an aryl, monocyclic ring containing 5 to 7 atoms of which 1, 2, 3 or 4 ring atoms are chosen from nitrogen, sulphur or oxygen. Examples of heteroarylene are oxazolylene, oxadiazolylene, pyridylene, pyrimidinylene, imidazolylene, triazolylene, tetrazolylene, pyrazinylene, pyridazinylene, pyrrolylene, thienylene and furylene.

Suitable optional substituents for heteroaryl groups, unless otherwise defined, are 1, 2 or 3 substituents independently selected from halo, cyano, nitro, amino, hydroxy, (1-4C)alkyl, (1-4C)alkoxy, (1-4C)alkylS(O)_{b} (wherein b is 0, 1 or 2), N-((1-4C)alkyl)amino and N,N-((1-4C)alkyl)₂amino. Further suitable optional substituents for “heteroaryl” groups are 1, 2 or 3 substituents independently selected from fluoro, chloro, cyano, nitro, amino, methylamino, dimethylamino, hydroxy, methyl, ethyl, methoxy, methylthio, methylsulfinyl and methylsulfonyl.

Preferred values of A, R¹ to R⁷ and n are as follows. Such values may be used where appropriate with any of the definitions, claims, aspects or embodiments defined hereinbefore or hereinafter.

In one embodiment of the invention are provided compounds of formula (1), in an alternative embodiment are provided pharmaceutically-acceptable salts of compounds of formula (1), in a further alternative embodiment are provided in-vivo hydrolysable esters of compounds of formula (1), and in a further alternative embodiment are provided pharmaceutically-acceptable salts of in-vivo hydrolysable esters of compounds of formula (1).

In one aspect of the present invention there is provided a compound of formula (1) as depicted above wherein R⁴ and R⁵ are together —S—C(R⁶)═C(R⁷)—.

In another aspect of the invention R⁴ and R⁵ are together —C(R⁷)═C(R⁶)—S—.

In a further aspect of the invention, R⁶ and R⁷ are independently selected from hydrogen, halo or (1-6C)alkyl.

Preferably R⁶ and R⁷ are independently selected from hydrogen, chloro, bromo or methyl.

Particularly R⁶ and R⁷ are independently selected from hydrogen or chloro.

More particularly one of R⁶ and R⁷ is chloro.

In one embodiment, one of R⁶ and R⁷ is chloro and the other is hydrogen.

In another embodiment, both R⁶ and R⁷ are chloro.

In one aspect of the invention A is phenylene.

In another aspect of the invention A is heteroarylene.

Preferably A is selected from phenylene, pyridylene, pyrimidinylene, pyrrolylene, imidazolylene, triazolylene, tetrazolylene, oxazolylene, oxadiazolylene, thienylene and furylene.

Further suitable values for A are phenylene, pyridylene, pyrimidinylene, pyrrolylene and imidazolylene.

Further suitable values for A are phenylene, pyridylene and pyrimidinylene.

Further suitable values for A are phenylene and pyridylene.

In one embodiment, when A is heteroarylene, there is a nitrogen in a bridgehead position. In another embodiment, when A is heteroarylene, the heteroatoms are not in bridgehead positions. It will be appreciated that the preferred (more stable) bridgehead position is as shown below:

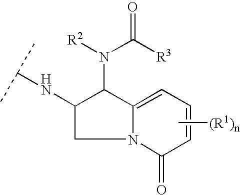

In one aspect of the invention n is 0 or 1.

In one aspect preferably n is 1.

In another aspect, preferably n is 0.

When n is 2, and the two R¹ groups, together with the carbon atoms of A to which they are attached, form a 4 to 7 membered saturated ring, optionally containing 1 or 2 heteroatoms independently selected from O, S and N, conveniently such a ring is a 5 or 6 membered ring. In one embodiment, such a 5 or 6 membered ring contains two O atoms (ie a cyclic acetal). When the two R¹ groups together form such a cyclic acetal, preferably it is not substituted. Most preferably the two R¹ groups together are the group —O—CH₂—O—.

In another aspect of the present invention R¹ is selected from halo, nitro, cyano, hydroxy, fluoromethyl, difluoromethyl, trifluoromethyl and (1-4C)alkoxy.

In a further aspect R¹ is selected from halo, nitro, cyano, hydroxy, fluoromethyl, difluoromethyl, trifluoromethyl, —S(O)_{b}(1-4C)alkyl (wherein b is 0, 1 or 2), —OS(O)₂(1-4C)alkyl, (1-4C)alkyl and (1-4C)alkoxy.

In a further aspect R¹ is selected from halo, nitro, cyano, hydroxy, fluoromethyl, difluoromethyl, trifluoromethyl, —S(O)_{b}Me (wherein b is 0, 1 or 2), —OS(O)₂Me, methyl and methoxy.

In a further aspect, R¹ is (1-4C)alkyl.

Preferably R¹ is selected from halo and (1-4C)alkoxy.

In another embodiment preferably R¹ is selected from fluoro, chloro, methyl, ethyl, methoxy and —O—CH₂—O—.

In one aspect R² is selected from R_{N}a where R_{N}a is selected from:
R_{N}a: (1-3C)alkyl, halo(1-3C)alkyl, dihalo(1-3)alkyl, trifluoromethyl, hydroxy(1-3C)alkyl, dihydroxy(2-3C)alkyl, cyano(1-3C)alkyl (optionally substituted on alkyl with hydroxy), methoxymethyl, ethoxymethyl, methoxyethyl, methoxymethoxymethyl, dimethoxyethyl, (hydroxy)(methoxy)ethyl, 5- and 6-membered acetals and mono- and di-methyl derivatives thereof, (amino)(hydroxy)(2-3C)alkyl, (aminocarbonyl)(hydroxy)(2-3C)alkyl, (methylaminocarbonyl)(hydroxy)(2-3C)alkyl, (dimethylaminocarbonyl)(hydroxy)(2-3C)alkyl, (methylcarbonylamino)(hydroxy)(2-3C)alkyl, (methylS(O)ₚ-)(hydroxy)(2-3C)alkyl (wherein p is 0, 1 or 2);
   and R³ is selected from R_{N}b where R_{N}b is selected from:
R_{N}b: (1-4C)alkyl, halo(1-4C)alkyl, dihalo(1-4C)alkyl, trifluoromethyl, hydroxy(1-4C)alkyl, dihydroxy(2-4C)alkyl, trihydroxy(3-4C)alkyl, cyano(1-4C)alkyl (optionally substituted on alkyl with hydroxy), (1-4C)alkoxy(1-4C)alkyl, (1-4C)alkoxy(1-4C)alkoxy(1-4C)alkyl, di[(1-4C)alkoxy](2-4C)alkyl, (hydroxy)[(1-4C)alkoxy](2-4C)alkyl, 5- and 6-membered acetals and mono- and di-methyl derivatives thereof, (amino)(hydroxy)(2-4C)alkyl, (aminocarbonyl)(hydroxy)(2-4C)alkyl, ((1-4C)alkylaminocarbonyl)(hydroxy)(2-4C)alkyl, (di(1-4C)alkylaminocarbonyl)(hydroxy)(2-4C)alkyl, ((1-4C)alkylcarbonylamino)(hydroxy)(2-4C)alkyl, ((1-4C)alkylS(O)ₚ-)(hydroxy)(2-4C)alkyl (wherein p is 0, 1 or 2);
   provided that when R_{N}a is (1-3C)alkyl, then R_{N}b is not (1-4C)alkyl.

In another aspect R² is selected from R_{N}a where R_{N}a is selected from
R_{N}a: (1-3C)alkyl, halo(1-3C)alkyl, dihalo(1-3)alkyl, trifluoromethyl, hydroxy(2-3C)alkyl, dihydroxy(2-3C)alkyl, cyano(1-3C)alkyl, methoxymethyl, ethoxymethyl, methoxyethyl, methoxymethoxymethyl, dimethoxyethyl, (hydroxy)(methoxy)ethyl, 5- and 6-membered acetals and mono- and di-methyl derivatives thereof;
   and R³ is selected from R_{N}b where R_{N}b is selected from:
R_{N}b: (1-4C)alkyl, halo(1-4C)alkyl, dihalo(1-4C)alkyl, trifluoromethyl, hydroxy(1-4C)alkyl, dihydroxy(2-4C)alkyl, trihydroxy(3-4C)alkyl, cyano(1-4C)alkyl, (1-4C)alkoxy(1-4C)alkyl, (1-4C)alkoxy(1-4C)alkoxy(1-4C)alkyl, di[(1-4C)alkoxy](2-4C)alkyl, (hydroxy)[(1-4C)alkoxy](1-4C)alkyl, 5- and 6-membered acetals and mono- and di-methyl derivatives thereof;
   provided that when R_{N}a is (1-3C)alkyl, then R_{N}b is not (1-4C)alkyl.

In another aspect R³ is selected from R_{N}a where R_{N}a is selected from
R_{N}a: (1-3C)alkyl, halo(1-3C)alkyl, dihalo(1-3)alkyl, trifluoromethyl, hydroxy(1-3C)alkyl, dihydroxy(2-3C)alkyl, cyano(1-3C)alkyl, methoxymethyl, ethoxymethyl, methoxyethyl, methoxymethoxymethyl, dimethoxyethyl, (hydroxy)(methoxy)ethyl, 5- and 6-membered acetals and mono- and di-methyl derivatives thereof;
   and R² is selected from R_{N}b where R_{N}b is selected from:
R_{N}b: (1-4C)alkyl, halo(1-4C)alkyl, dihalo(1-4C)alkyl, trifluoromethyl, hydroxy(2-4C)alkyl, dihydroxy(2-4C)alkyl, trihydroxy(3-4C)alkyl, cyano(1-4C)alkyl, (1-4C)alkoxy(1-4C)alkyl, (1-4C)alkoxy(1-4C)alkoxy(1-4C)alkyl, di[(1-4C)alkoxy](2-4C)alkyl, (hydroxy)[(1-4C)alkoxy](2-4C)alkyl, 5- and 6-membered acetals and mono- and di-methyl derivatives thereof;
   provided that when R_{N}a is (1-3C)alkyl, then R_{N}b is not (1-4C)alkyl.

In another aspect R² is selected from R_{N}a and R³ is selected from R_{N}b, wherein R_{N}a and R_{N}b are selected from any of the values for these groups defined hereinbefore or hereinafter.

In one embodiment, any alkyl or alkoxy group within any group in R_{N}A and R_{N}B is additionally substituted on an available carbon atom with a hydroxy group (provided that said carbon atom is not already substituted by a group linked by a heteroatom).

In another embodiment, any alkyl or alkoxy group within any group in R_{N}A and R_{N}B is not additionally substituted on an available carbon atom with a hydroxy group.

In one aspect, R_{N}a is selected from (1-3C)alkyl, halo(1-3C)alkyl, dihalo(1-3C)alkyl, trifluoromethyl, hydroxy(1-3C)alkyl, dihydroxy(2-3C)alkyl and cyano(1-3C)alkyl.

In one embodiment R_{N}a is selected from methyl, ethyl, fluoromethyl, chloromethyl, dichloromethyl, difluoromethyl, trifluoromethyl, hydroxymethyl, hydroxyethyl, hydroxypropyl, dihydroxy ethyl, dihydroxypropyl and cyanomethyl.

In another aspect R_{N}a is selected from (1-4C)alkyl, hydroxy(1-4C)alkyl, and (1-4C)alkoxy(1-4C)alkyl.

In another embodiment R_{N}a is selected from:
(1-3C)alkyl, halo(1-3C)alkyl, dihalo(1-3C)alkyl, trifluoromethyl, hydroxy(1-3C)alkyl, dihydroxy(2-3C)alkyl, cyano(1-3C)alkyl, methoxymethyl, ethoxymethyl, methoxyethyl, methoxymethoxymethyl, dimethoxyethyl and (hydroxy)(methoxy)ethyl.

In another embodiment R_{N}a is selected from:
methyl, ethyl, fluoromethyl, difluoromethyl, trifluoromethyl, hydroxymethyl, hydroxyethyl, dihydroxyethyl, dihydroxypropyl, methoxymethyl, methoxyethyl and dimethoxyethyl.

In another embodiment R_{N}a is selected from methyl, ethyl, hydroxymethyl, hydroxyethyl, dihydroxyethyl, and dihydroxypropyl.

In another embodiment R_{N}a is selected from methyl, ethyl, hydroxymethyl and hydroxyethyl.

In another embodiment R_{N}a is selected from methyl and hydroxyethyl.

In another embodiment R_{N}a is selected from methyl and ethyl.

In another embodiment R_{N}a is methyl.

In one embodiment R_{N}b is selected from hydroxy(1-4C)alkyl, dihydroxy(2-4C)alkyl, trihydroxy(3-4C)alkyl, cyano(1-4C)alkyl (substituted on alkyl with hydroxy), (1-4C)alkoxy(1-4C)alkyl, (1-4C)alkoxy(1-4C)alkoxy(1-4C)alkyl, di[(1-4C)alkoxy](2-4C)alkyl, (hydroxy)[(1-4C)alkoxy](2-4C)alkyl, 5- and 6-membered acetals and mono- and di-methyl derivatives thereof, (amino)(hydroxy)(2-4C)alkyl, (aminocarbonyl)(hydroxy)(1-4C)alkyl, ((1-4C)alkylaminocarbonyl)(hydroxy)(2-4C)alkyl, (di(1-4C)alkylaminocarbonyl)(hydroxy)(2-4C)alkyl, ((1-4C)alkylcarbonylamino)(hydroxy)(1-4C)alkyl, and ((1-4C)alkylS(O)ₚ-)(hydroxy)(1-4C)alkyl (wherein p is 0, 1 or 2).

In another embodiment R_{N}b is selected from hydroxy(1-4C)alkyl, dihydroxy(2-4C)alkyl, trihydroxy(1-4C)alkyl, (1-4C)alkoxy(1-4C)alkyl, (1-4C)alkoxy(1-4C)alkoxy(1-4C)alkyl, di[(1-4C)alkoxy](1-4C)alkyl, (hydroxy)[(1-4C)alkoxy](1-4C)alkyl, 5- and 6-membered acetals and mono- and di-methyl derivatives thereof.

In another embodiment R_{N}b is selected from hydroxy(1-4C)alkyl, dihydroxy(2-4C)alkyl, cyano(1-4C)alkyl (substituted on alkyl with hydroxy), (1-4C)alkoxy(1-4C)alkyl, (hydroxy)[(1-4C)alkoxy](1-4C)alkyl, (amino)(hydroxy)(1-4C)alkyl, (aminocarbonyl)(hydroxy)(1-4C)alkyl, ((1-4C)alkylaminocarbonyl)(hydroxy)(2-4C)alkyl, (di(1-4C)alkylaminocarbonyl)(hydroxy)(2-4C)alkyl, ((1-4C)alkylcarbonylamino)(hydroxy)(1-4C)alkyl, and ((1-4C)alkylS(O)ₚ-)(hydroxy)(1-4C)alkyl (wherein p is 0, 1 or 2).

In another embodiment R_{N}b is selected from hydroxy(1-4C)alkyl, dihydroxy(2-4C)alkyl, trihydroxy(3-4C)alkyl, 5- and 6-membered acetals and mono- and di-methyl derivatives thereof.

In another embodiment R_{N}b is selected from hydroxy(1-4C)alkyl and dihydroxy(2-4C)alkyl.

In another embodiment, R_{N}b is selected from dihydroxy(2-4C)alkyl and (hydroxy)[(1-4C)alkoxy](1-4C)alkyl.

In one aspect R_{N}b is selected from hydroxymethyl, hydroxyethyl, hydroxypropyl, dihydroxyethyl, 1,2-dihydroxypropyl, 2,3-dihydroxypropyl, 1,3-dihydroxypropyl, 1,2,3-trihydroxypropyl, methoxymethyl, methoxyethyl, methoxymethoxymethyl, dimethoxyethyl, hydroxyethoxyethyl, 3-dioxolan-4-yl, 2-methyl-1,3-dioxolan-4-yl, 2,2-dimethyl-1,3-dioxolan-4-yl; 2,2-dimethyl-1,3-dioxan-4-yl; 2,2-dimethyl-1,3-dioxan-5-yl; 1,3-dioxan-2-yl.

In another aspect R_{N}b is selected from hydroxymethyl, hydroxyethyl, hydroxypropyl, dihydroxyethyl, 1,2-dihydroxypropyl, 2,3-dihydroxypropyl, 1,3-dihydroxypropyl ,3-dioxolan-4-yl, 2-methyl-1,3-dioxolan-4-yl, 2,2-dimethyl-1,3-dioxolan-4-yl, 2,2-dimethyl-1,3-dioxan-4-yl, 2,2-dimethyl-1,3-dioxan-5-yl and 1,3-dioxan-2-yl.

In another aspect R_{N}b is selected from hydroxymethyl, hydroxyethyl, hydroxypropyl, dihydroxyethyl, 1,2-dihydroxypropyl, 2,3-dihydroxypropyl and 1,3-dihydroxypropyl.

In a further aspect, R_{N}b is selected from hydroxymethyl, 1-hydroxyethyl, 2-hydroxyethyl, hydroxypropyl, hydroxyisobutyl, dihydroxyethyl, 1,2-dihydroxypropyl, 2,3-dihydroxypropyl, 1,3-dihydroxypropyl, 1-(hydroxy)-2-(methoxy)ethyl, 1-(hydroxy)-2-(methylthio)ethyl, 1-(hydroxy)-2-(methylsulfonyl)ethyl, 1-(hydroxy)-2-(cyano)ethyl, 1-(hydroxy)-2-(amino)ethyl, 1-(amino)-2-(hydroxy)ethyl, 1-(hydroxy)-2-(aminocarbonyl)ethyl, 1-(hydroxy)-3-(aminocarbonyl)propyl, 1-(hydroxy)-2-(N-methylaminocarbonyl)ethyl, 1-(hydroxy)-2-(N,N-dimethylaminocarbonyl)ethyl and 1-(methylcarbonylamino)-2-(hydroxy)ethyl.

In a further aspect, R_{N}b is selected from hydroxymethyl, 1-hydroxyethyl, 2-hydroxyethyl, hydroxypropyl, hydroxyisobutyl, dihydroxyethyl, 1,2-dihydroxypropyl, 2,3-dihydroxypropyl, 1,3-dihydroxypropyl, 1-(hydroxy)-2-(methoxy)ethyl, 1-(hydroxy)-2-(methylthio)ethyl, 1-(hydroxy)-2-(methylsulfonyl)ethyl, 1-(hydroxy)-2-(cyano)ethyl, 1-(hydroxy)-2-(amino)ethyl and 1-(amino)-2-(hydroxy)ethyl.

In a further aspect, R_{N}b is selected from hydroxymethyl, 1-hydroxyethyl, 2-hydroxyethyl, hydroxypropyl, hydroxyisobutyl, dihydroxyethyl, 1,2-dihydroxypropyl, 2,3-dihydroxypropyl, 1,3-dihydroxypropyl, 1-(hydroxy)-2-(methoxy)ethyl, 1-(hydroxy)-2-(methylthio)ethyl and 1-(hydroxy)-2-(methylsulfonyl)ethyl.

In a further aspect, R_{N}b is selected from hydroxymethyl, 1-hydroxyethyl, 2-hydroxyethyl, hydroxypropyl, hydroxyisobutyl, dihydroxyethyl, 1,2-dihydroxypropyl, 2,3-dihydroxypropyl, 1,3-dihydroxypropyl and 1-(hydroxy)-2-(methoxy)ethyl.

In a further aspect, R_{N}b is selected from 1,2-dihydroxypropyl, 2,3-dihydroxypropyl, 1,3-dihydroxypropyl and 1-(hydroxy)-2-(methoxy)ethyl.

In one aspect of the invention is provided a compound of the formula (I) wherein
A is phenylene;
n is 0, 1 or 2;
R⁴ and R⁵ are together —S—C(R⁶)═C(R⁷)— or —C(R⁷)═C(R⁶)—S—;
R⁶ and R⁷ are independently selected from hydrogen, chloro, bromo or methyl;
R¹ is selected from fluoro, chloro, methyl, ethyl, methoxy and —O—CH₂—O—;
R² is selected from R_{N}a where R_{N}a is selected from
R_{N}a: (1-3C)alkyl, halo(1-3C)alkyl, dihalo(1-3)alkyl, trifluoromethyl, hydroxy(2-3C)alkyl, dihydroxy(2-3C)alkyl, cyano(1-3C)alkyl, methoxymethyl, ethoxymethyl, methoxyethyl, methoxymethoxymethyl, dimethoxyethyl, (hydroxy)(methoxy)ethyl, 5- and 6-membered acetals and mono- and di-methyl derivatives thereof;
and R³ is selected from R_{N}b where R_{N}b is selected from:
R_{N}b: (1-4C)alkyl, halo(1-4C)alkyl, dihalo(1-4C)alkyl, trifluoromethyl, hydroxy(1-4C)alkyl, dihydroxy(2-4C)alkyl, trihydroxy(3-4C)alkyl, cyano(1-4C)alkyl, (1-4C)alkoxy(1-4C)alkyl, (1-4C)alkoxy(1-4C)alkoxy(1-4C)alkyl, di[(1-4C)alkoxy](1-4C)alkyl, (hydroxy)[(1-4C)alkoxy](1-4C)alkyl, 5- and 6-membered acetals and mono- and di-methyl derivatives thereof;
provided that when R_{N}a is (1-3C)alkyl, then R_{N}b is not (1-4C)alkyl;
and pharmaceutically acceptable salts and in-vivo hydrolysable esters thereof.

In another aspect of the invention is provided a compound of the formula (I) wherein
A is heteroarylene;
n is 0, 1 or 2;
R⁴ and R⁵ are together —S—C(R⁶)═C(R⁷)— or —C(R⁷)═C(R⁶)—S—;
R⁶ and R⁷ are independently selected from hydrogen, chloro, bromo or methyl;
R¹ is selected from fluoro, chloro, methyl, ethyl, methoxy and —O—CH₂—O—;
R² is selected from R_{N}a where R_{N}a is selected from
R_{N}a: (1-3C)alkyl, halo(1-3C)alkyl, dihalo(1-3)alkyl, trifluoromethyl, hydroxy(2-3C)alkyl, dihydroxy(2-3C)alkyl, cyano(1-3C)alkyl, methoxymethyl, ethoxymethyl, methoxyethyl, methoxymethoxymethyl, dimethoxyethyl, (hydroxy)(methoxy)ethyl, 5- and 6-membered acetals and mono- and di-methyl derivatives thereof;
and R³ is selected from R_{N}b where R_{N}b is selected from:
R_{N}b: (1-4C)alkyl, halo(1-4C)alkyl, dihalo(1-4C)alkyl, trifluoromethyl, hydroxy(1-4C)alkyl, dihydroxy(2-4C)alkyl, trihydroxy(3-4C)alkyl, cyano(1-4C)alkyl, (1-4C)alkoxy(1-4C)alkyl, (1-4C)alkoxy(1-4C)alkoxy(1-4C)alkyl, di[(1-4C)alkoxy](1-4C)alkyl, (hydroxy)[(1-4C)alkoxy](1-4C)alkyl, 5- and 6-membered acetals and mono- and di-methyl derivatives thereof;
and pharmaceutically acceptable salts and in-vivo hydrolysable esters thereof.

In one aspect of the invention is provided a compound of the formula (I) wherein
A is phenylene;
n is 0, 1 or 2;
R⁴ and R⁵ are together —S—C(R⁶)═C(R⁷)— or —C(R⁷)═C(R⁶)—S—;
R⁶ and R⁷ are independently selected from hydrogen, chloro, bromo or methyl;
R¹ is selected from fluoro, chloro, methyl, ethyl, methoxy and —O—CH₂—O—;
R³ is selected from R_{N}a where R_{N}a is selected from
R_{N}a: (1-3C)alkyl, halo(1-3C)alkyl, dihalo(1-3)alkyl, trifluoromethyl, hydroxy(1-3C)alkyl, dihydroxy(2-3C)alkyl, cyano(1-3C)alkyl, methoxymethyl, ethoxymethyl, methoxyethyl, methoxymethoxymethyl, dimethoxyethyl, (hydroxy)(methoxy)ethyl, 5- and 6-membered acetals and mono- and di-methyl derivatives thereof;
and R² is selected from R_{N}b where R_{N}b is selected from:
R_{N}b: (1-4C)alkyl, halo(1-4C)alkyl, dihalo(1-4C)alkyl, trifluoromethyl, hydroxy(2-4C)alkyl, dihydroxy(2-4C)alkyl, trihydroxy(3-4C)alkyl, cyano(1-4C)alkyl, (1-4C)alkoxy(1-4C)alkyl, (1-4C)alkoxy(1-4C)alkoxy(1-4C)alkyl, di[(1-4C)alkoxy](1-4C)alkyl, (hydroxy)[(1-4C)alkoxy](1-4C)alkyl, 5- and 6-membered acetals and mono- and di-methyl derivatives thereof;
provided that when R_{N}a is (1-3C)alkyl, then R_{N}b is not (1-4C)alkyl;
and pharmaceutically acceptable salts and in-vivo hydrolysable esters thereof.

In another aspect of the invention is provided a compound of the formula (I) wherein
A is heteroarylene;
n is 0, 1 or 2;
R⁴ and R⁵ are together —S—C(R⁶)═C(R⁷)— or —C(R⁷)═C(R⁶)—S—;
R⁶ and R⁷ are independently selected from hydrogen, chloro, bromo or methyl;
R¹ is selected from fluoro, chloro, methyl, ethyl, methoxy and —O—CH₂—O—;
R³ is selected from R_{N}a where R_{N}a is selected from
R_{N}a: (1-3C)alkyl, halo(1-3C)alkyl, dihalo(1-3)alkyl, trifluoromethyl, hydroxy(1-3C)alkyl, dihydroxy(2-3C)alkyl, cyano(1-3C)alkyl, methoxymethyl, ethoxymethyl, methoxyethyl, methoxymethoxymethyl, dimethoxyethyl, (hydroxy)(methoxy)ethyl, 5- and 6-membered acetals and mono- and di-methyl derivatives thereof;
and R² is selected from R_{N}b where R_{N}b is selected from:
R_{N}b: (1-4C)alkyl, halo(1-4C)alkyl, dihalo(1-4C)alkyl, trifluoromethyl, hydroxy(2-4C)alkyl, dihydroxy(2-4C)alkyl, trihydroxy(3-4C)alkyl, cyano(1-4C)alkyl, (1-4C)alkoxy(1-4C)alkyl, (1-4C)alkoxy(1-4C)alkoxy(1-4C)alkyl, di[(1-4C)alkoxy](1-4C)alkyl, (hydroxy)[(1-4C)alkoxy](1-4C)alkyl, 5- and 6-membered acetals and mono- and di-methyl derivatives thereof;
and pharmaceutically acceptable salts and in-vivo hydrolysable esters thereof.

In another aspect of the invention is provided a compound of the formula (I) wherein
A is phenylene;
n is 0, 1 or 2;
R⁴ and R⁵ are together —S—C(R⁶)═C(R⁷)— or —C(R⁷)═C(R⁶)—S—;
R⁶ and R⁷ are independently selected from hydrogen, chloro, bromo or methyl;
R¹ is selected from fluoro, chloro, methyl, ethyl, methoxy and —O—CH₂—O—;
one of R² and R³ is selected from R_{N}a, and the other is selected from R_{N}b;
R_{N}a is selected from: (1-3C)alkyl, halo(1-3C)alkyl, dihalo(1-3C)alkyl, trifluoromethyl, hydroxy(1-3C)alkyl, dihydroxy(2-3C)alkyl, cyano(1-3C)alkyl, methoxymethyl, ethoxymethyl, methoxyethyl, methoxymethoxymethyl, dimethoxyethyl and (hydroxy)(methoxy)ethyl;
R_{N}b is selected from: hydroxy(1-4C)alkyl, dihydroxy(2-4C)alkyl, trihydroxy(1-4C)alkyl, (1-4C)alkoxy(1-4C)alkyl, (1-4C)alkoxy(1-4C)alkoxy(1-4C)alkyl, di[(1-4C)alkoxy](1-4C)alkyl, (hydroxy)[(1-4C)alkoxy](1-4C)alkyl, 5- and 6-membered acetals and mono- and di-methyl derivatives thereof;
and pharmaceutically acceptable salts and in-vivo hydrolysable esters thereof.

In another aspect of the invention is provided a compound of the formula (I) wherein
A is phenylene;
n is 0, 1 or 2;
R⁴ and R⁵ are together —S—C(R⁶)═C(R⁷)— or —C(R⁷)═C(R⁶)—S—;
R⁶ and R⁷ are independently selected from hydrogen, chloro, bromo or methyl;
R¹ is selected from fluoro, chloro, methyl, ethyl, methoxy and —O—CH₂—O—;
one of R² and R³ is selected from R_{N}a, and the other is selected from R_{N}b;
R_{N}a is selected from: methyl, ethyl, fluoromethyl, difluoromethyl, trifluoromethyl, hydroxymethyl, hydroxyethyl, dihydroxyethyl, dihydroxypropyl, methoxymethyl, methoxyethyl and dimethoxyethyl.
R_{N}b is selected from: hydroxymethyl, hydroxyethyl, hydroxypropyl, dihydroxyethyl, 1,2-dihydroxypropyl, 2,3-dihydroxypropyl, 1,3-dihydroxypropyl, 1,2,3-trihydroxypropyl, methoxymethyl, methoxyethyl, methoxymethoxymethyl, dimethoxyethyl, hydroxyethoxyethyl, 3-dioxolan-4-yl, 2-methyl-1,3-dioxolan-4-yl, 2,2-dimethyl-1,3-dioxolan-4-yl; 2,2-dimethyl-1,3-dioxan-4-yl; 2,2-dimethyl-1,3-dioxan-5-yl; 1,3-dioxan-2-yl;
and pharmaceutically acceptable salts and in-vivo hydrolysable esters thereof.

In another aspect of the invention is provided a compound of the formula (I) wherein
A is phenylene;
n is 0, 1 or 2;
R⁴ and R⁵ are together —S—C(R⁶)═C(R⁷)— or —C(R⁷)═C(R⁶)—S—;
R⁶ and R⁷ are independently selected from hydrogen, chloro, bromo or methyl;
R¹ is selected from fluoro, chloro, methyl, ethyl, methoxy and —O—CH₂—O—;
one of R² and R³ is selected from R_{N}a, and the other is selected from R_{N}b;
R_{N}a is selected from: methyl, ethyl, hydroxymethyl, hydroxyethyl, dihydroxyethyl, and dihydroxypropyl;
R_{N}b is selected from: hydroxymethyl, hydroxyethyl, hydroxypropyl, dihydroxyethyl, 1,2-dihydroxypropyl, 2,3-dihydroxypropyl, 1,3-dihydroxypropyl, 3-dioxolan-4-yl, 2-methyl-1,3-dioxolan-4-yl, 2,2-dimethyl-1,3-dioxolan-4-yl; 2,2-dimethyl-1,3-dioxan-4-yl; 2,2-dimethyl-1,3-dioxan-5-yl and 1,3-dioxan-2-yl;
and pharmaceutically acceptable salts and in-vivo hydrolysable esters thereof.

In another aspect of the invention is provided a compound of the formula (I) wherein
A is phenylene;
n is 0;
R⁴ and R⁵ are together —S—C(R⁶)═C(R⁷)— or —C(R⁷)═C(R⁶)—S—;
R⁶ and R⁷ are independently selected from hydrogen or chloro;
R² is (1-4C)alkyl;
one of R² and R³ is selected from R_{N}a, and the other is selected from R_{N}b;
R_{N}a is selected from: methyl and ethyl;
R_{N}b is selected from hydroxymethyl, hydroxyethyl, hydroxypropyl, dihydroxyethyl, 1,2-dihydroxypropyl, 2,3-dihydroxypropyl, and 1,3-dihydroxypropyl;
and pharmaceutically acceptable salts and in-vivo hydrolysable esters thereof.

In another aspect of the invention is provided a compound of the formula (I) wherein
A is phenylene;
n is 0, 1 or 2;
R⁴ and R⁵ are together —S—C(R⁶)═C(R⁷)— or —C(R⁷)═C(R⁶)—S—;
R⁶ and R⁷ are independently selected from hydrogen, chloro, bromo or methyl;
R¹ is selected from fluoro, chloro, methyl, ethyl, methoxy and —O—CH₂—O—;
R² is selected from R_{N}a where R_{N}a is selected from methyl, ethyl, hydroxymethyl and hydroxyethyl;
R³ is selected from R_{N}b where R_{N}b is selected from halo(1-4C)alkyl, dihalo(1-4C)alkyl, trifluoromethyl, hydroxy(1-4C)alkyl, dihydroxy(2-4C)alkyl, trihydroxy(3-4C)alkyl, cyano(1-4C)alkyl (optionally substituted on alkyl with hydroxy), (1-4C)alkoxy(1-4C)alkyl, (1-4C)alkoxy(1-4C)alkoxy(1-4C)alkyl, di[(1-4C)alkoxy](2-4C)alkyl, (hydroxy)[(1-4C)alkoxy](2-4C)alkyl, 5- and 6-membered acetals and mono- and di-methyl derivatives thereof, (amino)(hydroxy)(2-4C)alkyl, (aminocarbonyl)(hydroxy)(2-4C)alkyl, ((1-4C)alkylcarbonylamino)(hydroxy)(2-4C)alkyl, ((1-4C)alkylaminocarbonyl)(hydroxy)(2-4C)alkyl, (di(1-4C)alkylaminocarbonyl)(hydroxy)(2-4C)alkyl, ((1-4C)alkylS(O)ₚ-)(hydroxy)(2-4C)alkyl (wherein p is 0, 1 or 2);
and pharmaceutically acceptable salts and in-vivo hydrolysable esters thereof.

In another aspect of the invention is provided a compound of the formula (I) wherein
A is phenylene;
n is 0, 1 or 2;
R⁴ and R⁵ are together —S—C(R⁶)═C(R⁷)—or —C(R⁷)═C(R⁶)—S—;
R⁶ and R⁷ are independently selected from hydrogen, chloro, bromo or methyl;
R¹ is selected from fluoro, chloro, methyl, ethyl, methoxy and —O—CH₂—O—;
R² is selected from R_{N}a where R_{N}a is selected from methyl, ethyl, hydroxymethyl and hydroxyethyl;
R³ is selected from R_{N}b where R_{N}b is selected from hydroxy(1-4C)alkyl, dihydroxy(2-4C)alkyl, trihydroxy(3-4C)alkyl, cyano(1-4C)alkyl (substituted on alkyl with hydroxy), (1-4C)alkoxy(1-4C)alkyl, (1-4C)alkoxy(1-4C)alkoxy(1-4C)alkyl, di[(1-4C)alkoxy](2-4C)alkyl, (hydroxy)[(1-4C)alkoxy](2-4C)alkyl, 5- and 6-membered acetals and mono- and di-methyl derivatives thereof, (amino)(hydroxy)(2-4C)alkyl, (aminocarbonyl)(hydroxy)(2-4C)alkyl, ((1-4C)alkylaminocarbonyl)(hydroxy)(2-4C)alkyl, (di(1-4C)alkylaminocarbonyl)(hydroxy)(2-4C)alkyl, ((1-4C)alkylcarbonylamino)(hydroxy)(2-4C)alkyl, and ((1-4C)alkylS(O)ₚ-)(hydroxy)(2-4C)alkyl (wherein p is 0, 1 or 2);
and pharmaceutically acceptable salts and in-vivo hydrolysable esters thereof.

In another aspect of the invention is provided a compound of the formula (I) wherein
A is phenylene;
n is 0, 1 or 2;
R⁴ and R⁵ are together —S—C(R⁶)═C(R⁷)— or —C(R⁷)═C(R⁶)—S—;
R⁶ and R⁷ are independently selected from hydrogen, chloro, bromo or methyl;
R¹ is selected from fluoro, chloro, methyl, ethyl, methoxy and —O—CH₂—O—;
R² is selected from R_{N}a where R_{N}a is selected from methyl, ethyl, hydroxymethyl and hydroxyethyl;
R³ is selected from R_{N}b where R_{N}b is selected from hydroxy(1-4C)alkyl, dihydroxy(2-4C)alkyl, cyano(1-4C)alkyl (substituted on alkyl with hydroxy), (1-4C)alkoxy(1-4C)alkyl, (hydroxy)[(1-4C)alkoxy](2-4C)alkyl, (amino)(hydroxy)(2-4C)alkyl, (aminocarbonyl)(hydroxy)(2-4C)alkyl, ((1-4C)alkylaminocarbonyl)(hydroxy)(2-4C)alkyl, (di(1-4C)alkylaminocarbonyl)(hydroxy)(2-4C)alkyl, ((1-4C)alkylcarbonylamino)(hydroxy)(2-4C)alkyl, and ((1-4C)alkylS(O)ₚ-)(hydroxy)(2-4C)alkyl (wherein p is 0, 1 or 2);
and pharmaceutically acceptable salts and in-vivo hydrolysable esters thereof.

In another aspect of the invention is provided a compound of the formula (I) wherein
A is phenylene;
n is 0;
R⁴ and R⁵ are together —S—C(R⁶)═C(R⁷)— or —C(R⁷)═C(R⁶)—S—;
R⁶ and R⁷ are independently selected from hydrogen or chloro;
R² is selected from R_{N}a where R_{N}a is selected from methyl, ethyl, hydroxymethyl and hydroxyethyl;
R³ is selected from R_{N}b where R_{N}b is selected from hydroxy(1-4C)alkyl, dihydroxy(2-4C)alkyl, cyano(1-4C)alkyl (substituted on alkyl with hydroxy), (1-4C)alkoxy(1-4C)alkyl, (hydroxy)[(1-4C)alkoxy](2-4C)alkyl, (amino)(hydroxy)(2-4C)alkyl, (aminocarbonyl)(hydroxy)(2-4C)alkyl, ((1-4C)alkylaminocarbonyl)(hydroxy)(2-4C)alkyl, (di (1-4C)alkylaminocarbonyl)(hydroxy)(2-4C)alkyl, ((1-4C)alkylcarbonylamino)(hydroxy)(2-4C)alkyl, and ((1-4C)alkylS(O)ₚ-)(hydroxy)(2-4C)alkyl (wherein p is 0, 1 or 2);
and pharmaceutically acceptable salts and in-vivo hydrolysable esters thereof.

In another aspect of the invention is provided a compound of the formula (I) wherein
A is phenylene;
n is 0;
R⁴ and R⁵ are together —S—C(R⁶)═C(R⁷)— or —C(R⁷)═C(R⁶)—S—;
R⁶ and R⁷ are independently selected from hydrogen or chloro;
R² is selected from R_{N}a where R_{N}a is selected from methyl, ethyl, hydroxymethyl and hydroxyethyl;
R³ is selected from R_{N}b where R_{N}b is selected from hydroxy(1-4C)alkyl, dihydroxy(2-4C)alkyl, cyano(1-4C)alkyl (substituted on alkyl with hydroxy), (1-4C)alkoxy(1-4C)alkyl, (hydroxy)[(1-4C)alkoxy](2-4C)alkyl, (amino)(hydroxy)(2-4C)alkyl and ((1-4C)alkylS(O)ₚ-)(hydroxy)(2-4C)alkyl (wherein p is 0, 1 or 2);
and pharmaceutically acceptable salts and in-vivo hydrolysable esters thereof.

In another aspect of the invention is provided a compound of the formula (I) wherein
A is phenylene;
n is 0;
R⁴ and R⁵ are together —S—C(R⁶)═C(R⁷)— or —C(R⁷)═C(R⁶)—S—;
R⁶ and R⁷ are independently selected from hydrogen or chloro;
R² is selected from R_{N}a where R_{N}a is selected from methyl, ethyl, hydroxymethyl and hydroxyethyl;
R³ is selected from R_{N}b where R_{N}b is selected from hydroxymethyl, 1-hydroxyethyl, 2-hydroxyethyl, hydroxypropyl, hydroxyisobutyl, dihydroxyethyl, 1,2-dihydroxypropyl, 2,3-dihydroxypropyl, 1,3-dihydroxypropyl, 1-(hydroxy)-2-(methoxy)ethyl, 1-(hydroxy)-2-(methylthio)ethyl, 1-(hydroxy)-2-(methylsulfonyl)ethyl, 1-(hydroxy)-2-(cyano)ethyl, 1-(hydroxy)-2-(amino)ethyl, 1-(amino)-2-(hydroxy)ethyl, 1-(hydroxy)-2-(aminocarbonyl)ethyl, 1-(hydroxy)-3-(aminocarbonyl)propyl, 1-(hydroxy)-2-(N-methylaminocarbonyl)ethyl, 1-(hydroxy)-2-(N,N-dimethylaminocarbonyl)ethyl, 1-(methylcarbonylamino)-2-(hydroxy)ethyl;
and pharmaceutically acceptable salts and in-vivo hydrolysable esters thereof.

In another aspect of the invention is provided a compound of the formula (I) wherein
A is phenylene;
n is 0;
R⁴ and R⁵ are together —S—C(R⁶)═C(R⁷)— or —C(R⁷)═C(R⁶)—S—;
R⁶ and R⁷ are independently selected from hydrogen or chloro;
R² is selected from R_{N}a where R_{N}a is selected from methyl, ethyl, hydroxymethyl and hydroxyethyl;
R³ is selected from R_{N}b where R_{N}b is selected from hydroxymethyl, 1-hydroxyethyl, 2-hydroxyethyl, hydroxypropyl, hydroxyisobutyl, dihydroxyethyl, 1,2-dihydroxypropyl, 2,3-dihydroxypropyl, 1,3-dihydroxypropyl, 1-(hydroxy)-2-(methoxy)ethyl, 1-(hydroxy)-2-(methylthio)ethyl, 1-(hydroxy)-2-(methylsulfonyl)ethyl, 1-(hydroxy)-2-(cyano)ethyl, 1-(hydroxy)-2-(amino)ethyl and 1-(amino)-2-(hydroxy)ethyl;
and pharmaceutically acceptable salts and in-vivo hydrolysable esters thereof.

In another aspect of the invention is provided a compound of the formula (I) wherein
A is phenylene;
n is 0;
R⁴ and R⁵ are together —S—C(R⁶)═C(R⁷)— or —C(R⁷)═C(R⁶)—S—;
R⁶ and R⁷ are independently selected from hydrogen or chloro;
R² is selected from R_{N}a where R_{N}a is selected from methyl, ethyl, hydroxymethyl and hydroxyethyl;
R³ is selected from R_{N}b where R_{N}b is selected from hydroxymethyl, 1-hydroxyethyl, 2-hydroxyethyl, hydroxypropyl, hydroxyisobutyl, dihydroxyethyl, 1,2-dihydroxypropyl, 2,3-dihydroxypropyl, 1,3-dihydroxypropyl, 1-(hydroxy)-2-(methoxy)ethyl, 1-(hydroxy)-2-(methylthio)ethyl and 1-(hydroxy)-2-(methylsulfonyl)ethyl;
and pharmaceutically acceptable salts and in-vivo hydrolysable esters thereof.

In another aspect of the invention is provided a compound of the formula (I) wherein
A is phenylene;
n is 0;
R⁴ and R⁵ are together —S—C(R⁶)═C(R⁷)— or —C(R⁷)═C(R⁶)—S—;
R⁶ and R⁷ are independently selected from hydrogen or chloro;
R² is selected from R_{N}a where R_{N}a is selected from methyl, ethyl, hydroxymethyl and hydroxyethyl;
R³ is selected from R_{N}b where R_{N}b is selected from hydroxymethyl, 1-hydroxyethyl, 2-hydroxyethyl, hydroxypropyl, hydroxyisobutyl, dihydroxyethyl, 1,2-dihydroxypropyl, 2,3-dihydroxypropyl, 1,3-dihydroxypropyl and 1-(hydroxy)-2-(methoxy)ethyl;
and pharmaceutically acceptable salts and in-vivo hydrolysable esters thereof.

In another aspect of the invention is provided a compound of the formula (I) wherein
A is phenylene;
n is 0;
R⁴ and R⁵ are together —S—C(R⁶)═C(R⁷)— or —C(R⁷)═C(R⁶)—S—;
R⁶ and R⁷ are independently selected from hydrogen or chloro;
R² is selected from R_{N}a where R_{N}a is selected from methyl, ethyl, hydroxymethyl and hydroxyethyl;
R³ is selected from R_{N}b where R_{N}b is selected from 1,2-dihydroxypropyl, 2,3-dihydroxypropyl, 1,3-dihydroxypropyl and 1-(hydroxy)-2-(methoxy)ethyl;
and pharmaceutically acceptable salts and in-vivo hydrolysable esters thereof.

Preferred compounds of the invention are of the formula (1A), wherein R¹ to R⁵ and n are as defined in any aspect or embodiment described hereinbefore or hereinafter.

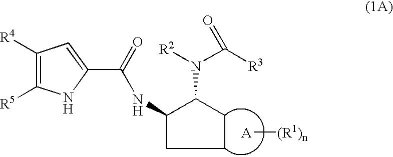

In one aspect, preferred compounds of the invention are compounds of the formula (1) or (1A) as defined hereinbefore or hereinafter wherein R³ contains an hydroxy group on the carbon adjacent to the carbonyl group. Further preferred compounds of the invention are compounds of the formula (1) or (1A) as defined hereinbefore or hereinafter wherein R³ contains an amino group on the carbon adjacent to the carbonyl group.

Particular compounds of the invention are each of the Examples, each of which provides a further independent aspect of the invention. In a further aspect of the invention there is provided any two or more of the Examples or a pharmaceutically acceptable salt or pro-drug thereof.

Another aspect of the present invention provides a process for preparing a compound of formula (1) or a pharmaceutically acceptable salt or an in-vivo hydrolysable ester thereof which process (wherein A, R¹ to R⁵ and n are, unless otherwise specified, as defined in formula (1)) comprises of:
a) reacting an acid of the formula (2): 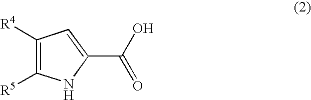
   or an activated derivative thereof; with an amine of formula (3): 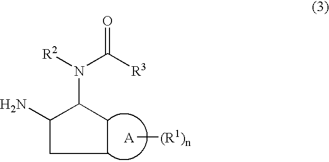
   and thereafter if necessary:
i) converting a compound of the formula (1) into another compound of the formula (1);
ii) removing any protecting groups;
iii) forming a pharmaceutically acceptable salt or in-vivo hydrolysable ester.

Specific reaction conditions for the above reaction are as follows.

Process a)

Acids of formula (2) and amines of formula (3) may be coupled together in the presence of a suitable coupling reagent. Standard peptide coupling reagents known in the art can be employed as suitable coupling reagents, or for example carbonyldiimidazole, 1-ethyl-3-(3-dimethylaminopropyl)carbodiimide hydrochloride (EDCI) and dicyclohexyl-carbodiimide (DCCI), optionally in the presence of a catalyst such as 1-hydroxybenzotriazole, dimethylaminopyridine or 4-pyrrolidinopyridine, optionally in the presence of a base for example triethylamine, di-isopropylethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Suitable activated acid derivatives include acid halides, for example acid chlorides, and active esters, for example pentafluorophenyl esters. The reaction of these types of compounds with amines is well known in the art, for example they may be reacted in the presence of a base, such as those described above, and in a suitable solvent, such as those described above. The reaction may conveniently be performed at a temperature in the range of −40 to 40° C.

A compounds of formula (2) may be prepared according to Scheme 1:

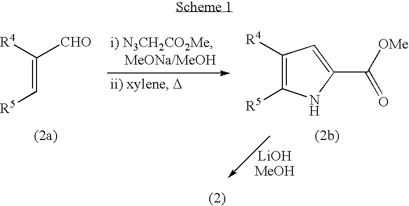

Compounds of formula (2a) are commercially available or they are known compounds or they are prepared by processes known in the art.

Compounds of formula (2b) may also be prepared as illustrated in Scheme 2: 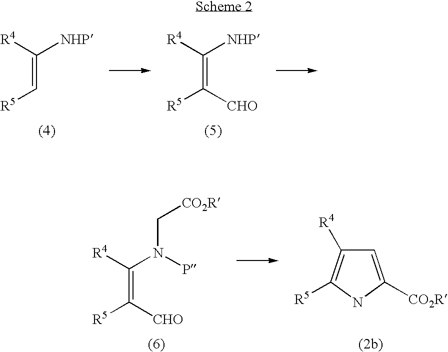
The conversion of compounds of formula (4) into compounds of formula (5) may be carried out by directed ortho lithiation reactions (J. Org. Chem, 2001, volume 66, 3662-3670), for example with n-butyl lithium and (CHO)N(alkyl)₂. The protecting group P′ in compounds of formula (4) must be suitable directing group for this reaction and may be for example —CO₂tBu. Reaction of compounds of formula (5) with LCH₂CO₂R where L is a leaving group, and replacement of the protecting group P′ with an alternative P″ (for example —COalkyl) according to standard processes, gives a compound of formula (6). This may be cyclised using a base, for example potassium carbonate or sodium methoxide.

Compounds of formula (3) may be prepared according to Scheme 3:

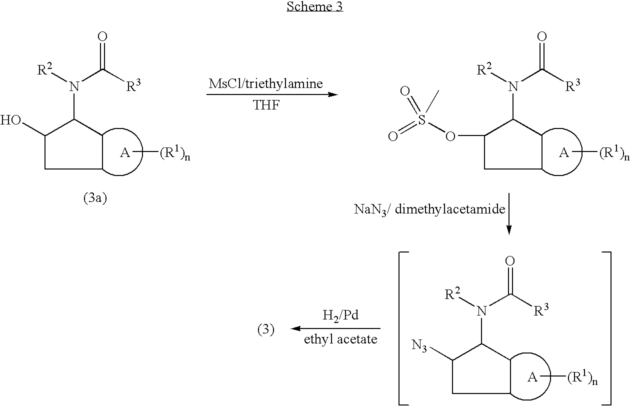

Compounds of formula (3a) are commercially available or they are known compounds or they are prepared by processes known in the art. For example, starting from primary amines of formula (7), in which R is H or a suitable protecting group, one or both of R² and/or R³ may be introduced by acylation, (for example reacting with acetoxyacetic acid and 1-(3-dimethylaminopropyl)-3-ethyl-carbodiimide hydrochloride-EDAC), alkylation, reductive alkylation, sulphonation or related processes, followed by O-deprotection when appropriate. Alternatively, one or both of R² and/or R³ may be obtained by modification of functionality in groups previously thus introduced, by reduction, oxidation, hydrolysis (for example the conversion of an acetoxy group to a hydroxy group), nucleophilic displacement, amidation, or a related process, or a combination of these processes, followed by O-deprotection when appropriate. It will be appreciated that such modifications may include modifications which convert one compound of the formula (1) into another compound of the formula (1).

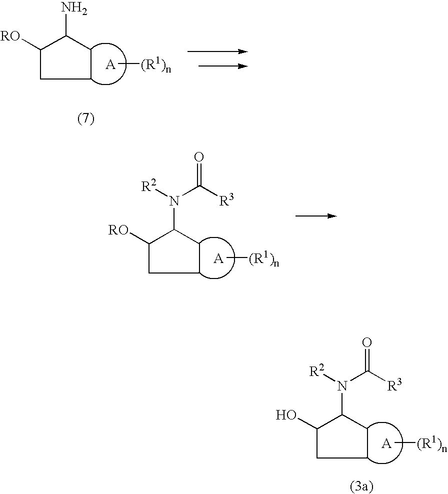

Amines of formula (3) may alternatively be obtained by applying the processes described for the preparation of compounds of formula (3a) to compounds of formula (8) in which W is NH₂ or a nitrogen atom with one or two suitable protecting groups.

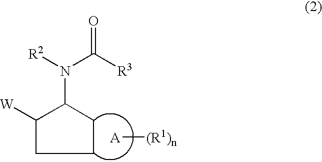

Alternatively, amines of formula (3) may also be prepared by the process in Scheme 3A. Compounds of formula A are commercially available or they are known compounds or they are prepared by processes known in the art. For example compound A can be converted to the phthalamido-protected intermediate C under standard conditions (Step 1). Alkylation can then be performed under standard conditions (Step 2: NaH, MeI, DMA). Removal of the phthalamide then affords amine D (Step 3; hydrazine hydrate, EtOH).

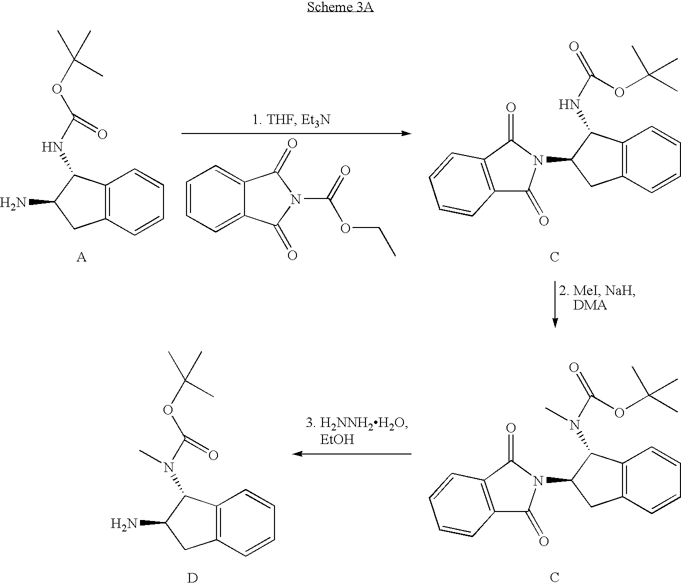

Compounds of the formula (3) where r=1 and wherein A is heteroarylene can be prepared from suitably functionalised cycloalkyl fused heterocycles. For example, when A is pyridine, 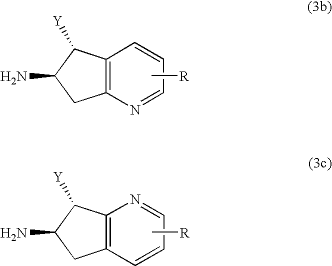
compounds of formula (3b) and (3c) may be prepared from the corresponding azaindanone regioisomer according to Scheme 4:

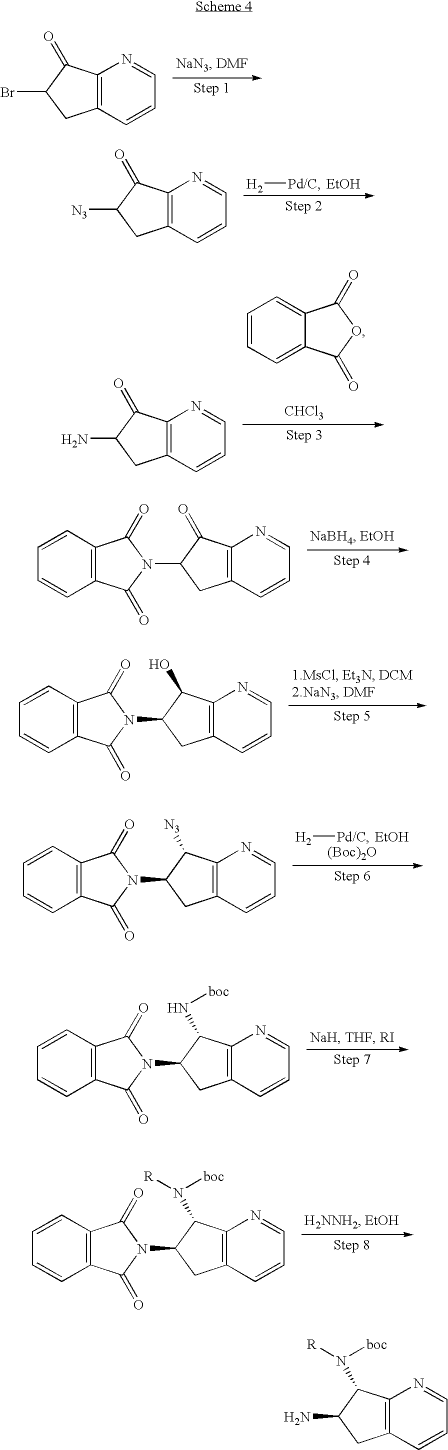

Step 1 is performed on a compound known in the literature (Jpn. Kokai Tokkyo Koho, 1995, 14. JP 07070136). Steps 2, 3, 4, 5, 6, 7 and 8 are performed using standard techniques known in the art.

It will be appreciated that the bromoazaindanone isomers (21a, 21b and 21c) could 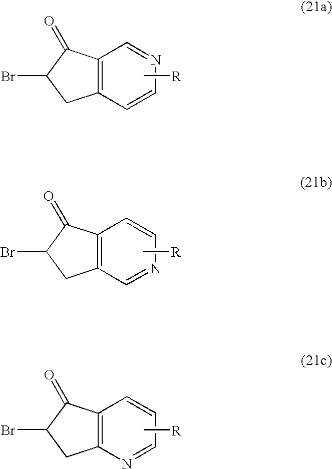
be converted to the corresponding heterocylic version of (3) by the means described in Scheme 4. The bromoazaindanone can be prepared from the corresponding azaindanone by standard techniques known in the art. The azaindanone (22a, 22b, 22c) are known in the literature or they are prepared by processes known in the art.

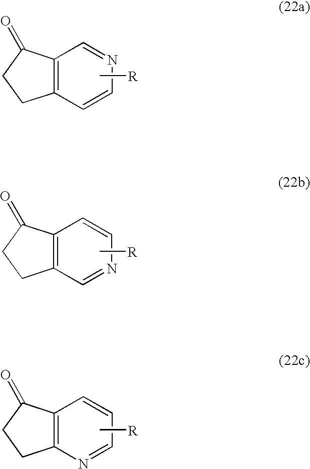

The process described above and shown in Scheme 4 may also be applied to other six membered heterocycles containing more than one nitrogen.

It will be appreciated that, in a similar manner, compounds of the formula (3) wherein A is heteroarylene containing a bridgehead nitrogen can be prepared from the appropriate suitably functionalised cycloalkyl fused heterocycles.

It will be appreciated that the processes described above for formation and modification of —NR²C(O)R³ may be applied similarly whether to make the compound of formula (3) before coupling to the acid of formula (2) or whether to the product of such a coupling.

It will be appreciated that certain of the various ring substituents in the compounds of the present invention, for example R¹ may be introduced by standard aromatic substitution reactions or generated by conventional functional group modifications either prior to or immediately following the processes mentioned above, and as such are included in the process aspect of the invention. Such reactions may convert one compound of the formula (1) into another compound of the formula (1). Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogen group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Certain intermediates in the preparation of a compound of the formula (1) are novel and form another aspect of the invention.

Compounds of the invention generally possess improved physical properties (for example solubility and/or plasma-protein binding) in comparison with those of the compounds previously disclosed, which are particularly beneficial for a pharmaceutical. In combination with glycogen phosphorylase inhibitory activity, such physical properties render the compounds of the invention particularly useful as pharmaceuticals.

For example, the compounds of the invention generally show improved solubility in comparison with the equivalent compounds where R² is H. This effect is illustrated by the thermodynamic solubilities of Examples 8 and 17, and Reference Example 1 given in the table below.

| Ex- | | Solu- | Ac- |
|---|---|---|---|
| am- | | bil- | tivity |
| ple | | ity | (μM) |
| No | Structure | (μM) | IC₅₀ |
| 8 | 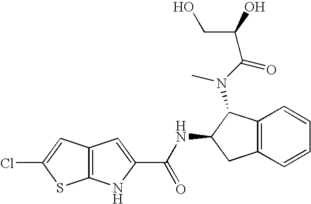 | 176 | 0.07 |
| 27 | 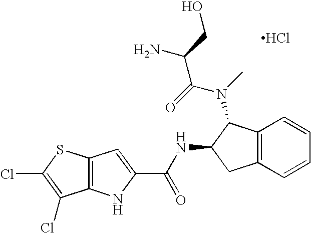 | 50 | 0.11 |
| Ref- er- ence Eg 1 | 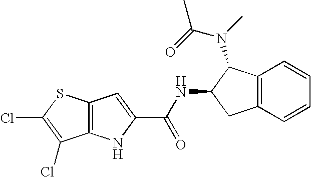 | 1.4 | 0.4 ap- prox |

For Reference Example 1, See WO 02/20530 example 154.

The thermodynamic solubility data for the compounds of the invention as given above may be measured by agitating the compound in 0.1 M phosphate at pH7.4 for 24 hours, then analysis of the supernatant (for example by LCUV/MS) using a solution (for example in DMSO) of known concentration as the calibrant.

Plasma Protein binding may be measured using an equilibrium dialysis technique, whereby compound is added to 10% plasma giving a concentration of 20 μM and dialysed with isotonic buffer for 18 hours at 37° C. The plasma and buffer solutions are analysed using LCUVMS and the first apparent binding constant for the compound derived. The binding constant is then used to determine the % free in 100% plasma.

The binding constant derived from the dialysis experiment is based upon a model of 1:1 binding between compound and albumin.

$$P + D ⇌ P D$$

$$K 1 = \frac{[ P D ]}{[ P ] \times [ D ]}$$

where P=free protein, D=free drug, PD=drug protein complex, K1=first apparent binding constant.

As stated hereinbefore the compounds defined in the present invention possesses glycogen phosphorylase inhibitory activity. This property may be assessed by, for example, using the procedure set out below.

Assay

The activity of the compounds is determined by measuring the inhibitory effect of the compounds on glycogen degradation, the production of glucose-1-phosphate from glycogen is monitored by the multienzyme coupled assay, as described in EP 0 846 464 A2, general method of Pesce et al (Pesce, M A, Bodourian, S H, Harris, R C, and Nicholson, J F (1977) Clinical Chemistry 23, 1171-1717). The reactions were in 384 well microplate format in a volume of 50 μl. The change in fluorescence due to the conversion of the co-factor NAD to NADH is measured at 340 nM excitation, 465 nm emission in a Tecan Ultra Multifunctional Microplate Reader. The reaction is in 50 mM HEPES, 3.5 mM KH₂PO₄, 2.5 mM MgCl₂, 2.5 mM ethylene glycol-bis(b-aminoethyl ether) N,N,N′,N′-tetraacetic acid, 100 mM KCl, 8 mM D-(+)-glucose pH7.2, containing 0.5 mM dithiothreitol, the assay buffer solution. Human recombinant liver glycogen phosphorylase a (hrl GPa) 20 nM is pre-incubated in assay buffer solution with 6.25 mM NAD, 1.25 mg type III glycogen at 1.25 mg ml⁻¹ the reagent buffer, for 30 minutes. The coupling enzymes, phosphoglucomutase and glucose-6-phosphate dehydrogenase (Sigma) are prepared in reagent buffer, final concentration 0.25 Units per well. 20 μl of the hrl GPa solution is added to 10 μl compound solution and the reaction started with the addition of 20 μl coupling enzyme solution. Compounds to be tested are prepared in 10 μl 5% DMSO in assay buffer solution, with final concentration of 1% DMSO in the assay. The non-inhibited activity of GPa is measured in the presence of 10 μl 5% DMSO in assay buffer solution and maximum inhibition measured in the presence of 5 mgs ml⁻¹ N-ethylmaleimide. After 6 hours at 30° C. Relative Fluoresence Units (RFUs) are measured at 340 nM excitation, 465 nm emission.

The assay is performed at a test concentration of inhibitor of 10 μM or 100 μM. Compounds demonstrating significant inhibition at one or both of these concentrations may be further evaluated using a range of test concentrations of inhibitor to determine an IC₅₀, a concentration predicted to inhibit the enzyme reaction by 50%.

Activity is calculated as follows:—
% inhibition=(1−(compound RFUs−fully inhibited RFUs)/(non-inhibited rate RFUs−fully inhibited RFUs))*100.

Typical IC₅₀ values for compounds of the invention when tested in the above assay are in the range 100 μM to 1 nM. For example, Example 1 was found to have an IC₅₀ of 265 nM and Example 8 to have an IC₅₀ of 176 nm.

The inhibitory activity of compounds was further tested in rat primary hepatocytes. Rat hepatocytes were isolated by the collagenase perfusion technique, general method of Seglen (P. O. Seglen, Methods Cell Biology (1976) 13 29-83). Cells were cultured on Nunclon six well culture plates in DMEM (Dulbeco's Modified Eagle's Medium) with high level of glucose containing 10% foetal calf serum, NEAA (non essential amino acids), Glutamine, penicillin/streptomycin ((100 units/100 μg)/ml) for 4 to 6 hours. The hepatocytes were then cultured in the DMEM solution without foetal calf serum and with 10 nM insulin and 10 nM dexamethasone. Experiments were initiated after 18-20 hours culture by washing the cells and adding Krebs-Henseleit bicarbonate buffer containing 2.5 mM CaCl₂ and 1% gelatin. The test compound was added and 5 minutes later the cells were challenged with 25 nM glucagon. The Krebs-Henseleit solution was removed after 60 min incubation at 37° C., 95% O₂/5% CO₂ and the glucose concentration of the Krebs-Henseleit solution measured.

According to a further aspect of the invention there is provided a pharmaceutical composition which comprises a compound of the formula (1), or a pharmaceutically acceptable salt or in vivo hydrolysable ester thereof, as defined hereinbefore in association with a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents. In one aspect, the compositions of the invention are a form suitable for oral dosage.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The compound of formula (1) will normally be administered to a warm-blooded animal at a unit dose within the range 5-5000 mg per square meter body area of the animal, i.e. approximately 0.1-100 mg/kg, and this normally provides a therapeutically-effective dose. A unit dose form such as a tablet or capsule will usually contain, for example 1-250 mg of active ingredient. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

The inhibition of glycogen phosphorylase activity described herein may be applied as a sole therapy or may involve, in addition to the subject of the present invention, one or more other substances and/or treatments. Such conjoint treatment may be achieved by way of the simultaneous, sequential or separate administration of the individual components of the treatment. Simultaneous treatment may be in a single tablet or in separate tablets. For example, in order to prevent, delay or treat type 2 diabetes mellitus, the compounds of the present invention or their pharmaceutically acceptable salts may be administered in combination with one or more of the following agent(s):
1) Insulin and insulin analogues;
2) Insulin secretagogues including sulphonylureas (for example glibenclamide, glipizide), prandial glucose regulators (for example repaglinide, nateglinide) and glucokinase activators
3) Agents that improve incretin action (for example dipeptidyl peptidase IV inhibitors, GLP-1 agonists)
4) Insulin sensitising agents including PPARgamma agonists (for example pioglitazone and rosiglitazone); and agents with combined PPARalpha and gamma activity
5) Agents that modulate hepatic glucose balance (for example metformin, fructose 1, 6 bisphosphatase inhibitors, glycogen synthase kinase inhibitors, glucokinase activators)
6) Agents designed to reduce the absorption of glucose from the intestine (for example acarbose);
7) Agents that prevent the reabsorption of glucose by the kidney (SGLT inhibitors)
8) Agents designed to treat the complications of prolonged hyperglycaemia (for example aldose reductase inhibitors)
9) Anti-obesity agents (for example sibutramine and orlistat);
10) Anti-dyslipidaemia agents such as, HMG-CoA reductase inhibitors (statins, eg pravastatin); PPARα agonists (fibrates, eg gemfibrozil); bile acid sequestrants (cholestyramine); cholesterol absorption inhibitors (plant stanols, synthetic inhibitors); bile acid absorption inhibitors (IBATi) and nicotinic acid and analogues (niacin and slow release formulations);
11) Antihypertensive agents such as, β blockers (eg atenolol, inderal); ACE inhibitors (eg lisinopril); Calcium antagonists (eg. nifedipine); Angiotensin receptor antagonists (eg candesartan), α antagonists and diuretic agents (eg. furosemide, benzthiazide);
12) Haemostasis modulators such as, antithrombotics, activators of fibrinolysis and antiplatelet agents; thrombin antagonists; factor Xa inhibitors; factor VIIa inhibitors); antiplatelet agents (eg. aspirin, clopidogrel); anticoagulants (heparin and Low molecular weight analogues, hirudin) and warfarin;
13) Agents which antagonise the actions of glucagon; and
14) Anti-inflammatory agents, such as non-steroidal anti-inflammatory drugs (eg. aspirin) and steroidal anti-inflammatory agents (eg. cortisone).

According to a further aspect of the present invention there is provided a compound of the formula (1), or a pharmaceutically acceptable salt or in vivo hydrolysable ester thereof, as defined hereinbefore, for use in a method of treatment of a warm-blooded animal such as man by therapy.

According to an additional aspect of the invention there is provided a compound of the formula (1), or a pharmaceutically acceptable salt or in vivo hydrolysable ester thereof, as defined hereinbefore, for use as a medicament.

According to an additional aspect of the invention there is provided a compound of the formula (1), or a pharmaceutically acceptable salt or in vivo hydrolysable ester thereof, as defined hereinbefore, for use as a medicament in the treatment of type 2 diabetes, insulin resistance, syndrome X, hyperinsulinaemia, hyperglucagonaemia, cardiac ischaemia or obesity in a warm-blooded animal such as man.

According to this another aspect of the invention there is provided the use of a compound of the formula (1), or a pharmaceutically acceptable salt or in-vivo hydrolysable ester thereof, as defined hereinbefore in the manufacture of a medicament for use in the treatment of type 2 diabetes, insulin resistance, syndrome X, hyperinsulinaemia, hyperglucagonaemia, cardiac ischaemia or obesity in a warm-blooded animal such as man.

According to this another aspect of the invention there is provided the use of a compound of the formula (1), or a pharmaceutically acceptable salt or in-vivo hydrolysable ester thereof, as defined hereinbefore in the manufacture of a medicament for use in the treatment of type 2 diabetes in a warm-blooded animal such as man.

According to a further feature of this aspect of the invention there is provided a method of producing a glycogen phosphorylase inhibitory effect in a warm-blooded animal, such as man, in need of such treatment which comprises administering to said animal an effective amount of a compound of formula (1).

According to this further feature of this aspect of the invention there is provided a method of treating type 2 diabetes, insulin resistance, syndrome X, hyperinsulinaemia, hyperglucagonaemia, cardiac ischaemia or obesity in a warm-blooded animal, such as man, in need of such treatment which comprises administering to said animal an effective amount of a compound of formula (1).

According to this further feature of this aspect of the invention there is provided a method of treating type 2 diabetes in a warm-blooded animal, such as man, in need of such treatment which comprises administering to said animal an effective amount of a compound of formula (1).

As stated above the size of the dose required for the therapeutic or prophylactic treatment of a particular cell-proliferation disease will necessarily be varied depending on the host treated, the route of administration and the severity of the illness being treated. A unit dose in the range, for example, 1-100 mg/kg, preferably 1-50 mg/kg is envisaged.

In addition to their use in therapeutic medicine, the compounds of formula (1) and their pharmaceutically acceptable salts are also useful as pharmacological tools in the development and standardisation of in vitro and in vivo test systems for the evaluation of the effects of inhibitors of cell cycle activity in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated by the following examples in which, unless stated otherwise:
(i) temperatures are given in degrees Celsius (° C.); operations were carried out at room or ambient temperature, that is, at a temperature in the range of 18-25° C. and under an atmosphere of an inert gas such as argon;
(ii) organic solutions were dried over anhydrous magnesium sulphate; evaporation of solvent was carried out using a rotary evaporator under reduced pressure (600-4000 Pascals; 4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC and reaction times are given for illustration only;
(v) yields are given for illustration only and are not necessarily those which can be obtained by diligent process development; preparations were repeated if more material was required;
(vi) where given, NMR data is in the form of delta values for major diagnostic protons, given in parts per million (ppm) relative to tetramethylsilane (TMS) as an internal standard, determined at 300 MHz using perdeuterio dimethyl sulphoxide (DMSO-δ₆) as solvent unless otherwise indicated, other solvents (where indicated in the text) include deuterated chloroform CDCl₃;
(vii) chemical symbols have their usual meanings; SI units and symbols are used;
(viii) reduced pressures are given as absolute pressures in Pascals (Pa); elevated pressures are given as gauge pressures in bars;
(ix) solvent ratios are given in volume: volume (v/v) terms;
(x) mass spectra (MS) were run with an electron energy of 70 electron volts in the chemical ionisation (CI) mode using a direct exposure probe; where indicated ionisation was effected by electron impact (EI), fast atom bombardment (FAB) or electrospray (ESP); values for m/z are given; generally, only ions which indicate the parent mass are reported and unless otherwise stated the value quoted is (M−H)⁻;
(xi) The following abbreviations may be used:
   SM starting material;
   EtOAc ethyl acetate;
   MeOH methanol;
   EtOH ethanol;
   DCM dichloromethane;
   HOBT 1-hydroxybenzotriazole;
   DIPEA di-isopropylethylamine;
   EDCI 1-ethyl-3-(3-dimethylaminopropyl)carbodiimide hydrochloride;
   EDAC 1-ethyl-3-(3-dimethylaminopropyl)carbodiimide hydrochloride;
   Et₂O/ether diethyl ether;
   THF tetrahydrofuran;
   DMF N,N-dimethylformamide;
   HATU O-(7-Azabenzotriazol-1-yl)-N,N,N′, N′-tetramethyluroniumhexafluorophosphate
   EDAC 1-(3-dimethylaminopropyl)-3-ethyl-carbodiimide hydrochloride
   TFA Trifluoroacetic acid
   DMTMM 4-(4,6-Dimethoxy-1,3,5-triazin-2-yl)-4-methylmorpholinium chloride
   DMA N,N-dimethylacetamide

### Example 1

2-Chloro-N-{(1R,2R)-1-[(methoxyacetyl)(methyl)amino]-2,3-dihydro-1H-inden-2-yl}-6H-thieno[2,3-b]pyrrole-5-carboxamide

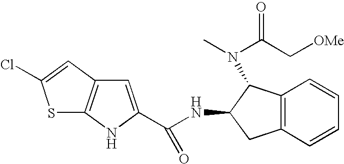

DIPEA (171 μL, 1.0 mmol), HOBT (54 mg, 0.4 mmol), methoxyacetic acid (31 μL, 0.4 mmol) and EDAC (96 mg, 0.5 mmol) were added to a suspension of 2-chloro-N-[(1R,2R)-1-(methylamino)-2,3-dihydro-1H-inden-2-yl]-6H-thieno[2,3-b]pyrrole-5-carboxamide hydrochloride (Intermediate 1, 150 mg, 0.4 mmol) in anhydrous DCM (5 mL). The reaction was stirred at ambient temperature for approximately 6 h. The volatiles were removed by evaporation under reduced pressure, the residue dissolved in EtOAc (20 mL), washed with saturated aqueous NaHCO₃ (20 mL), water (2×10 mL) then brine (10 mL) and dried (MgSO₄). The volatiles were removed by evaporation under reduced pressure and the residue triturated (EtOAc:hexane, 1:10), collected by filtration, washed with hexane (2×5 mL) and dried to give the title compound (126 mg, 75%) as a brown solid.

¹H NMR (300 MHz, d₆-DMSO) δ: 2.61 (s, 1.5H), 2.74 (s, 1.5H), 2.98 (m, 1H), 3.18 (dd, 1H), 3.19 (s, 1.5H), 3.22 (s, 1.5H), 3.99 (d, 0.5H), 4.13 (q, 1H), 4.31 (d, 0.5H), 4.79 (m, 1H), 5.41 (d, 0.5H), 6.09 (d, 0.5H), 7.12 (m, 6H), 8.52 (ap t, 1H), 11.81 (br s, 0.5H), 11.89 (br s, 0.5H); MS m/z 418, 420.

The following examples were prepared in a similar manner to Example 1 using 2-chloro-N-[(1R,2R)-1-(methylamino)-2,3-dihydro-1H-inden-2-yl]-6H-thieno[2,3-b]pyrrole-5-carboxamide hydrochloride (Intermediate 1) as the amine and the appropriate commercially available carboxylic acid as the coupling partner:

There is no Example with Example No 2.

### Example 3

2-Chloro-N-{(1R,2R)-1-[[3-hydroxy-2-(hydroxymethyl)propanoyl](methyl)amino]-2,3-dihydro-1H-inden-2-yl}-6H-thieno[2,3-b]pyrrole-5-carboxamide

### Example 4

Ethyl 3-[((1R,2R)-2-{[(2-chloro-6H-thieno[2,3-b]pyrrol-5-yl)carbonyl]amino}-2,3-dihydro-1H-inden-1-yl)(methyl)amino]-3-oxopropanoate

### Example 5

2-[((1R,2R)-2-{[(2-Chloro-6H-thieno[2,3-b]pyrrol-5-yl)carbonyl]amino}-2,3-dihydro-1H-inden-1-yl)(methyl)amino]-2-oxoethyl acetate

### Example 6

2-Chloro-N-{(1R,2R)-1-[glycoloyl(methyl)amino]-2,3-dihydro-1H-inden-2-yl}-6H-thieno[2,3-b]pyrrole-5-carboxamide

### Example 7

2-Chloro-N-{(1R,2R)-1-[glyceroyl(methyl)amino]-2,3-dihydro-1H-inden-2-yl}-6H-thieno[2,3-b]pyrrole-5-carboxamide

### Example 8

2-Chloro-N-{(1R,2R)-1-[[(2S)-2,3-dihydroxypropanoyl](methyl)amino]-2,3-dihydro-1H-inden-2-yl}-6H-thieno[2,3-b]pyrrole-5-carboxamide

### Example 9

2-Chloro-N-{(1R,2R)-1-[[(2R)-2,3-dihydroxypropanoyl](methyl)amino]-2,3-dihydro-1H-inden-2-yl}-6H-thieno[2,3-b]pyrrole-5-carboxamide

Note: Example 4 is an example of a pro-drug of a compound containing a carboxylic acid group and Example 5 is an example of a pro-drug of a compound containing a hydroxy group.

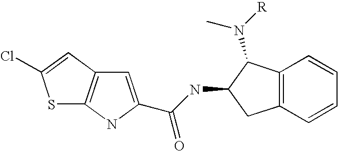

| Ex- | | | |
|---|---|---|---|
| am- | | | |
| ple | R | ¹H NMR (300 MHz, d₆-DMSO) | M/z |
| 3 | 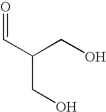 | δ: 2.61(s, 1.5H), 2.87(s, 1.5H), 2.99(m, 1H), 3.18(m, 2H), 3.41(m, 1H), 3.61(m, 2H), 4.44(t, 0.5H), 4.49(t, 0.5H), 4.66(m, 1H), 4.80(m, 1H), 5.74(d, 0.5H), 6.18(0.5H, d), 6.97(m, 2H), 7.21(m, 4H), 8.47(m, 1H), 11.78(br s, 0.5H), 11.84(br s, 0.5H) | 446, 448 (M − H)⁻ |
| 4 | 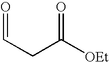 | δ: 1.07(t, 1.5H), 1.11(t, 1.5H), 2.61(s, 1.5H), 2.79(s, 1.5H), 2.97(m, 1H), 3.20(dd, 1H), 3.42(m, 1H), 3.71(d, 0.5H), 3.68(d, 0.5H), 4.00(q, 1H), 4.06(q, 1H), 4.79(m, 1H), 5.41(d, 0.5H), 6.11(d, 0.5H), 7.11(m, 6H), 8.53(m, 1H), 11.82(br s, 0.5H), 11.91(br s, 0.5H) | 460, 462 |
| 5 | 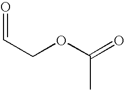 | δ: 2.02(s, 1.5H), 2.08(s, 1.5H), 2.61(s, 1.5H), 2.77(s, 1.5H), 2.98(dd, 1H), 3.21(dd, 1H, 4.75(m, 2H), 4.97(m, 1H), 5.33(d, 0.5H), 6.05(d, 0.5H), 7.08(m, 3H), 7.25(m, 3H), 8.55(dd, 1H), 11.82(s, 0.5H), 11.9(s, 0.5H). | 446, 448 |
| 6 | 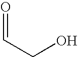 | δ: 2.65(s, 1.5H), 2.74(s, 1.5H), 3.0(m, 1H), 3.21(m, 1H), 4.17(m, 2H), 4.45(t, 0.5H), 4.65(m, 0.5H), 4.8(m, 1H), 5.34(d, 0.5H), 6.14(d, 0.5H), 7.05(m, 2H), 7.17(d, 1H), 7.28(m, 3H), 8.55(m, 1H), 11.87(d, 1H). | 402, 404 |
| 7 | 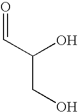 | δ(CDCl₃): 2.92(m, 4H), 3.35(m, 2H), 3.95(m, 2H), 4.78(m, 3H), 6.24(dd, 1H), 6.6(m, 3H), 7.1(m, 3.5), 7.8(m, 0.5H), 10.42(s, 1H), 10.86(s, 0.5H). | 456, 458 |
| 8 | 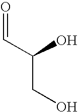 | 2.73(s, 1.5H), 2.97(s, 1.5H), 3.05(m, 1H), 3.3(m, 1H), 3.6(m, 2H), 4.75(m, 4H), 5.74(d, 0.5H), 6.18(d, 0.5H), 7.15(m, 3H), 7.35(m, 3H), 8.6(m, 1H), 11.83(s, 0.5H), 11.88(s, 0.5H) | 456, 458 |
| 9 | 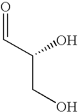 | δ: 2.7(s, 1.5H), 2.95(s, 1.5H), 3.05(m, 1H), 3.31(m, 1H), 3.6(m, 2H), 4.5(m, 1.5H), 4.77(d, 0.5H), 4.9(m, 1.5H), 5.38(d, 0.5H), 5.82(d, 0.5H), 6.24(d, 0.5H), 7.13(m, 3H), 3.34(m, 3H), 8.62(d, 1H), 11.86(s, 1H) | 456, 458 |

There is no Example with Example No 10.

### Example 11

2-Chloro-N-{(1R,2R)-1-[(3-hydroxypropanoyl)(methyl)amino]-2,3-dihydro-1H-inden-2-yl}-6H-thieno[2,3-b]pyrrole-5-carboxamide

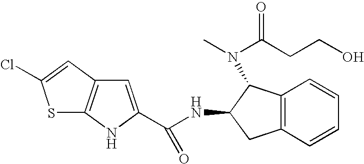

LiBH₄ (14 mg, 0.67 mmol) was added to a solution of ethyl 3-[((1R,2R)-2-{[(2-chloro-6H-thieno[2,3-b]pyrrol-5-yl)carbonyl]amino}-2,3-dihydro-1H-inden-1-yl)(methyl)amino]-3-oxopropanoate (Example 4; 103 mg, 0.22 mmol) at 0° C. The reaction was warmed and stirred at ambient temperature for 3 hours. Saturated aqueous NH₄Cl (10 mL) was added and the aqueous phase was extracted with EtOAc (3×20 mL). The combined organic phases were washed with HCl (2N, 20 mL), saturated aqueous NaHCO₃ (20 mL), water (20 mL) and brine (20 mL) then dried (MgSO₄) and the solvent was removed under reduced pressure. The crude product was purified by flash column chromatography (SiO₂, EtOAc eluent) to afford the title compound (62 mg, 67%) as a solid.

¹H NMR (300 MHz, d₆-DMSO) δ: 2.43 (m, 1H,), 2.60 (s, 1.5H), 2.70 (m, 1H), 2.80 (s, 1.5H), 2.97 (dd, 1H), 3.19 (dd, 1H), 3.62 (m, 2H), 4.46 (t, 0.5H), 4.52 (t, 0.5H), 4.78 (m, 1H), 5.55 (d, 0.5H), 6.14 (d, 0.5H), 7.12 (m, 6H), 8.49 (d, 0.5H), 8.54 (d, 0.5H), 11.82 (br s, 0.5H), 11.89 (br s, 0.5H); MS m/z 418, 420.

### Example 12

2-Chloro-N-{(1R,2R)-1-[glycoloyl(2-hydroxyethyl)amino]-2,3-dihydro-1H-inden-2-yl}-6H-thieno[2,3-b]pyrrole-5-carboxamide

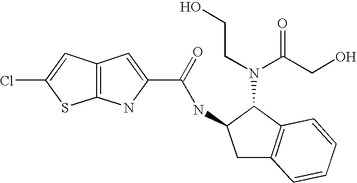

LiBH₄ (3.0 ml, 2M in THF, 6.0 mmol) was added to a solution of ethyl N-[(acetyloxy)acetyl]-N-((1R,2R)-2-{[(2-chloro-6H-thieno[2,3-b]pyrrol-5-yl)carbonyl]amino}-2,3-dihydro-1H-inden-1-yl)glycinate (Intermediate 11; 620 mg, 1.2 mmol) in dry THF (25 ml) at 5° C. and stirred for 30 mins. The reaction was allowed to warm to ambient temperature, stirred for 20 hours, HCl (1N, 10 ml) added and the aqueous phase extracted with EtOAc (50 ml). The combined organic phases were washed with water (20 mL) and brine (20 mL) then dried (MgSO₄) and the solvent was removed under reduced pressure. The crude product was purified by flash column chromatography (SiO₂, EtOAc:hexane eluent) to afford the title compound (230 mg, 44%) as a foam.

¹H NMR (300 MHz, d₆-DMSO) δ: 2.9 (m, 2H), 3.37 (m, 3H), 4.25 (m, 3H), 4.75 (m, 3H), 5.35 (d, 0.6H), 5.59 (m, 0.4H), 7.0 (s, 1H), 7.2 (m, 5H), 8.54 (m, 1H), 11.84 (m, 1H); MS m/z 432, 434.

### Example 13

2-Chloro-N-{(1R,2R)-1-[[(2R)-2-hydroxypropanoyl](methyl)amino]-2,3-dihydro-1H-inden-2-yl}-6H-thieno[2,3-b]pyrrole-5-carboxamide

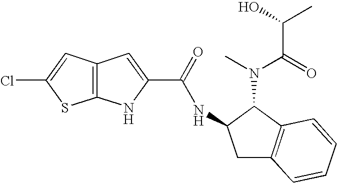

To a solution of 2-chloro-N-[(1R,2R)-1-(methylamino)-2,3-dihydro-1H-inden-2-yl]-6H-thieno[2,3-b]pyrrole-5-carboxamide hydrochloride (Intermediate 1; 191mg, 0.5 mmol), (R)-lactic acid (45 mg, 0.5 mmol) and DIPEA (171 μL, 1.0 mmol) in anhydrous DMA (5 mL) was added HOBT (68 mg, 0.5 mmol) and EDCI (120 mg, 0.625 mmol). The reaction was stirred at ambient temperature for approximately 24 h, water (25 mL) was added and the resulting precipitate filtered. The solid was purified by flash column chromatography (EtOAc:hexane, 1:1) to give the title compound (110 mg, 52%).

¹H NMR (mixture of rotamers) δ: 1.1 (d, 1.5H), 1.22 (d, 1.5H), 2.64 (s, 1.5H), 2.83 (s, 1.5H), 3.0 (m, 1H), 3.2 (m, 1H), 4.5 (m, 1H), 4.8 (m, 2H), 5.8 (d, 0.5H), 6.1 (d, 0.5H), 7.2 (m, 6H), 8.55 (m, 1H), 11.85 (m, 1H); MS m/z 416, 418 (M−H).

The following example was made by the process of Example 13, using 2-chloro-N-[(1R,2R)-1-(methylamino)-2,3-dihydro-1H-inden-2-yl]-6H-thieno[2,3-b]pyrrole-5-carboxamide hydrochloride (Intermediate 1) as the amine salt and the (S)-lactic acid as the carboxylic acid.

### Example 14

2-Chloro-N-{(1R,2R)-1-[[(2S)-2-hydroxypropanoyl](methyl)amino]-2,3-dihydro-1H-inden-2-yl}-6H-thieno[2,3-b]pyrrole-5-carboxamide

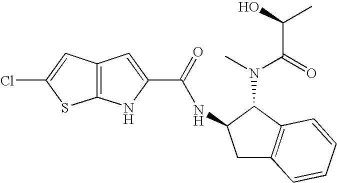

¹H NMR (mixture of rotamers) δ: 1.1 (d, 1.5H), 1.22 (d, 1.5H), 2.64 (s, 1.5H), 2.83 (s, 1.5H), 3.0 (m, 1H), 3.2 (m, 1H), 4.5 (m, 1H), 4.8 (m, 2H), 5.7 (d, 0.5H), 6.15 (d, 0.5H), 7.2 (m, 6H), 8.55 (m, 1H), 11.85 (m, 1H); MS m/z 416, 418 (M−H)⁻.

### Example 15

2,3-Dichloro-N-{(1R,2R)-1-[[(2R)-2,3-dihydroxypropanoyl](methyl)amino]-2,3-dihydro-1H-inden-2-yl}-4H-thieno[3,2-b]pyrrole-5-carboxamide

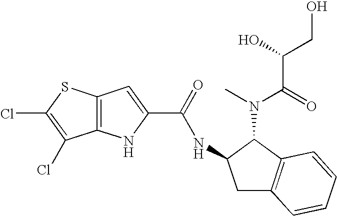

2,3-Dichloro-N-{(1R,2R)-1-[{[(4R)-2,2-dimethyl-1,3-dioxolan-4-yl]carbonyl}(methyl)amino]-2,3-dihydro-1H-inden-2-yl}-4H-thieno[3,2-b]pyrrole-5-carboxamide (Intermediate 20; 285 mg, 0.56 mmol) was dissolved in acetic acid (4 mL, 20% aqueous) and warmed to 70° C. for 3 h. The reaction was cooled, water (20 mL) added and the mixture filtered, the solid washed with water and dried in vacuo to give the title compound (200 mg, 76%) as a powder.

¹H NMR (mixture of rotamers) δ: 2.65 (s, 1.5H), 2.9 (s, 1.5H), 3.0 (m, 1H), 3.25 (m, 1H), 3.5 (m, 2H), 4.65 (m, 4H), 5.65 (d, 0.5H), 6.1 (d, 0.5H), 7.05 (m, 2H), 7.25 (m, 3H), 8.63 (m, 1H), 12.4 (m, 1H); MS m/z 466, 468 (M−H).

The following example was made by the process of Example 15, using 2,3-Dichloro-N-{(1R,2R)-1-[{[(4S)-2,2-dimethyl-1,3-dioxolan-4-yl]carbonyl}(methyl)amino]-2,3-dihydro-1H-inden-2-yl}-4H-thieno[3,2-b]pyrrole-5-carboxamide (Intermediate 21) as the acetonide.

### Example 16

2,3-Dichloro-N-{(1R,2R)-1-[[(2S)-2,3-dihydroxypropanoyl](methyl)amino]-2,3-dihydro-1H-inden-2-yl}-4H-thieno[3,2-b]pyrrole-5-carboxamide

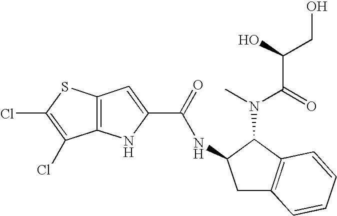

¹H NMR (mixture of rotamers) δ: 2.62 (s, 1.5H), 2.87 (s, 1.5H), 2.98 (m, 1H), 3.2 (m, 1H), 3.5 (m, 2H), 4.4 (m, 1.5H), 4.8 (m, 2H), 5.3 (m, 0.5H), 5.75 (d, 0.5H), 6.2 (d, 0.5H), 7.2 (m, 5H), 8.65 (d, 1H), 12.4 (m, 1H); MS m/z 466, 468 (M−H).

### Example 17

(2S)-N¹-((1R,2R)-2-{[(2-Chloro-6H-thieno[2,3-b]pyrrol-5-yl)carbonyl]amino}-2,3-dihydro-1H-inden-1-yl)-2-hydroxy-N¹-methylsuccinamide

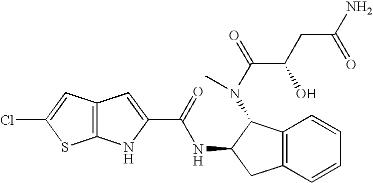

(2S)-4-Amino-2-hydroxy-4-oxobutanoic acid (CAS Reg. No.: 57229-74-0, 109 mg, 0.82 mmol), 2-chloro-N-[(1R,2R)-1-(methylamino)-2,3-dihydro-1H-inden-2-yl]-6H-thieno[2,3-b]pyrrole-5-carboxamide hydrochloride (Intermediate 1; 284 mg, 0.74 mmol), HOBT (111 mg, 0.82 mmol) and triethylamine (0.46 mL, 3.3 mmol) were suspended in DMF (5 mL) and stirred at ambient temperature. EDCI (157 mg, 0.82 mmol) was added and stirring was continued at ambient temperature for a further 18 hours. The reaction mixture was purified by reverse phase HPLC (5-95% acetonitrile/water gradient containing 0.2% TFA) to give the title compound (34 mg, 10%) as a white solid.

¹H NMR δ: 2.43 (m, 2H), 2.75 (d, 3H), 2.97 (m, 1H), 3.22 (m, 1H), 4.77 (m, 2H), 5.90 (dd, 1H), 6.81 (d, 1H), 7.16 (m, 7H), 8.58 (dd, 1H), 11.85 (s, 1H); MS m/z 461.1.

The following examples were made by the process of Example 17 using 2,3-dichloro-N-[(1R,2R)-1-(methylamino)-2,3-dihydro-1H-inden-2-yl]-4,6a-dihydro-3aH-thieno[3,2-b]pyrrole-5-carboxamide hydrochloride (Intermediate 16) as the amine source and the appropriate commercially available carboxylic acid.

### Example 18

(2S)-N¹-((1R,2R)-2-{[(2,3-Dichloro-4H-thieno[3,2-b]pyrrol-5-yl)carbonyl]amino}-2,3-dihydro-1H-inden-1-yl)-2-hydroxy-N¹-methylsuccinamide

### Example 19

2,3-dichloro-N-{(1R,2R)-1-[[(2S)-2-hydroxybutanoyl](methyl)amino]-2,3-dihydro-1H-inden-2-yl}-4H-thieno[3,2-b]pyrrole-5-carboxamide

### Example 20

2,3-Dichloro-N-{(1R,2R)-1-[[(2S)-2-hydroxy-3-methylbutanoyl](methyl)amino]-2,3-dihydro-1H-inden-2-yl}-4H-thieno[3,2-b]pyrrole-5-carboxamide

### Example 21

2,3-Dichloro-N-{(1R,2R)-1-[[(2S)-4-(1,3-dioxo-1,3-dihydro-2H-isoindol-2-yl)-2-hydroxybutanoyl](methyl)amino]-2,3-dihydro-1H-inden-2-yl}-4H-thieno[3,2-b]pyrrole-5-carboxamide

### Example 22

2,3-Dichloro-N-{(1R,2R)-1-[[(2R)-2-hydroxy-3-(methylthio)propanoyl](methyl)amino]-2,3-dihydro-1H-inden-2-yl}-4H-thieno[3,2-b]pyrrole-5-carboxamide

### Example 23

tert-Butyl {(2S)-3-[((1R,2R)-2-{[(2,3-dichloro-4H-thieno[3,2-b]pyrrol-5-yl)carbonyl]amino}-2,3-dihydro-1H-inden-1-yl)(methyl)amino]-2-hydroxy-3-oxopropyl}carbamate

### Example 24

2,3-dichloro-N-{(1R,2R)-1-[[(2S)-3-cyano-2-hydroxypropanoyl](methyl)amino]-2,3-dihydro-1H-inden-2-yl}-4H-thieno[3,2-b]pyrrole-5-carboxamide

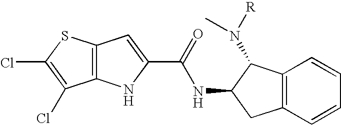

| Example | | | |
|---|---|---|---|
| No | R Group | ¹H NMR δ: | MS |
| 18 | 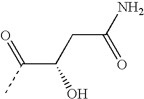 | 2.48(m, 2H), 2.75(d, 3H), 2.97(m, 1H), 3.22(m, 1H), 4.77(m, 2H), 5.90(dd, 1H), 6.81(d, 1H), 7.14(m, 6H), 8.67(dd, 1H), 12.39(s, 1H), MS m/z 493.2. | 493.2 |
| 19 | 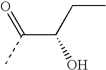 | 0.61(t, 3H), 0.90(t, 3H), 1.54(m, 2H), 2.73(d,3H), 3.00(m, 1H); 3.23(m, 1H), 4.26(m, 1H), 4.88(m, 1H), 5.91(dd, 1H), 6.98(m, 2H), 7.26(m, 3H), 8.69(dd, 1H), 12.44(d, 1H) | 466.0 |
| 20 | 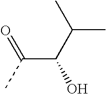 | 0.63(dd, 3H), 0.87(t, 3H), 1.94(m, 1H), 2.73(d, 3H), 3.00(m, 1H), 3.23(m, 1H), 4.05(dd, 1H), 4.88(m, 1H), 5.91(dd, 1H), 6.98(m, 2H), 7.26(m, 3H), 8.67(dd, 1H), 12.44(d, 1H) | 480.2 |
| 22 | 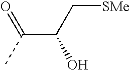 | 2.26(s, 3H), 2.91(m, 7H), 3.51(m, 1H), 4.75(m, 2H), 6.30(d, 1H), 6.54(s, 1H), 7.01(d, 1H), 7.22(m, 3H), 10.14(s, 1H) | 498.2 |
| Intermediate 30 | 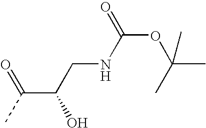 | 1.27(d, 9H), 2.73(d, 3H), 2.99(m, 1H), 3.19(m, 2H), 4.41(m, 1H), 4.85(m, 2H), 5.62(dd, 1H), 6.14(d, 1H), 7.08(m, 5H), 8.63(m, 1H), 12.40(d, 1H) | 564.5 |
| 24 | 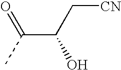 | 2.90(m, 4H), 3.07(s, 3H), 3.41(m, 1H), 4.88(m, 2H), 6.25(s, 1H), 6.37(d, 1H), 6.68(d, 1H) 7.03(d, 3H), 7.23(m, 2H), 10.58(s, 1H) | 475.1 |

There is no Example 21 or 23.

### Example 25

N-{(1R,2R)-1-[(N-Acetylseryl)(methyl)amino]-2,3-dihydro-1H-inden-2-yl}-2-chloro-6H-thieno[2,3-b]pyrrole-5-carboxamide

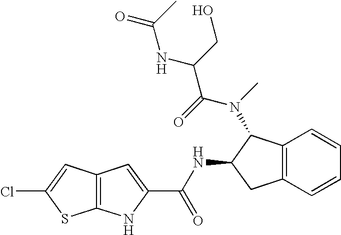

DIPEA (307 μL, 1.8 mmol), HOBT (74 mg, 0.55 mmol), N-acetylserine (74 mg, 0.5 mmol) and EDAC (115 mg, 0.6 mmol) were added to a suspension of 2-chloro-N-[(1R,2R)-1-(methylamino)-2,3-dihydro-1H-inden-2-yl]-6H-thieno[2,3-b]pyrrole-5-carboxamide hydrochloride (Intermediate 1; 191 mg, 0.5 mmol) in anhydrous DMF (4 mL). The reaction was stirred at ambient temperature for approximately 16 h and then diluted with water (20 mL). The resulting crude product was recovered by filtration and purified by reverse phase preparative HPLC (C18 ODS column, acetonitrile/water gradient 5-95% containing 0.2% TFA eluant) to give the title compound (35 mg, 8%).

¹H NMR δ: 1.8 (m, 3H), 3.0 (m, 7H), 4.9 (m, 2H), 5.7 (dd, 0.5H), 6.15 (dd, 0.5H), 7.1 (m, H), 8.25 (m, 2H), 11.8 (m, 1H); MS m/z 473, 475.

The following compound was prepared by the process of Example 25 using 2,3-dichloro-N-[(1R,2R)-1-(methylamino)-2,3-dihydro-1H-inden-2-yl]-4H-thieno[3,2-b]pyrrole-5-carboxamide hydrochloride (Intermediate 16) as the amine and N-acetylserine as the carboxylic acid.

### Example 26

N-{(1R,2R)-1-[(N-Acetylseryl)(methyl)amino]-2,3-dihydro-1H-inden-2-yl}-2,3-dichloro-4H-thieno[3,2-b]pyrrole-5-carboxamide

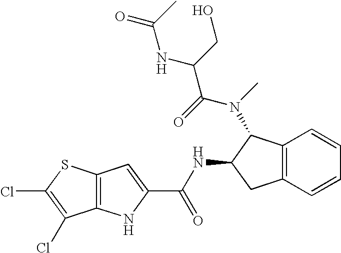

¹H NMR δ: 1.75 (m, 3H), 3.0 (m, 7H), 4.8 (m, 2H), 5.7 (dd, 0.5H), 6.11 (dd, 0.5H), 7.2 (m, 5H), 8.3 (m, 2H), 12.35 (m, 1H); MS m/z 507, 509.

### Example 27

2,3-Dichloro-N-{(1R,2R)-1-[methyl(L-seryl)amino]-2,3-dihydro-1H-inden-2-y}-4H-thieno[3,2-b]pyrrole-5-carboxamide hydrochloride

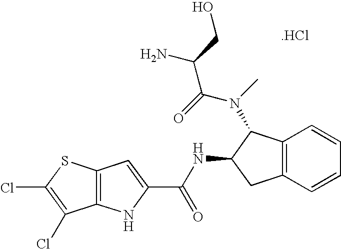

DIPEA (266 μL, 1.53 mmol), HOBT (101 mg, 0.75 mmol), N-(tert-butoxycarbonyl)-L-serine (103 mg, 0.5 mmol) and EDAC (119 mg, 0.62 mmol) were added to a suspension of 2,3-dichloro-N-[(1R,2R)-1-(methylamino)-2,3-dihydro-1H-inden-2-yl]-4H-thieno[3,2-b]pyrrole-5-carboxamide hydrochloride (Intermediate 16; 208 mg, 0.5 mmol) in anhydrous DMF (4 mL). The reaction was stirred at ambient temperature for approximately 16 h then diluted with water (20 mL) and the precipitated solid recovered by filtration and dried under vacuum. The crude material was purified by chromatography on silica gel eluting with an isohexane/EtOAc gradient (0-80%) and then dissolved in 4M HCl/Dioxan and left to stand for 1 h at ambient temperature. The volatiles were removed by evaporation under reduced pressure and the resulting gum triturated with ether to give the title compound (119 mg, 51%) as a white solid.

¹H NMR δ:2.7 (s, 1.5H), 2.9 (s, 1.5H), 3.1 (m, 1H), 3.25 (m, 1H), 3.8 (m, 2H), 4.4 (m, 1H), 5.0 (m, 1H), 5.6 (m, 1H), 5.75 (d, 0.5H), 6.15 (d, 0.5H), 7.1 (m, 5H), 8.1 (m, 3H), 8.8 (d, 0.5H), 9.3 (d, 0.5H), 12.52 (d, 1H); MS m/z 467.

The following example was prepared by the method of Example 27 using 2-chloro-N-[(1R,2R)-1-(methylamino)-2,3-dihydro-1H-inden-2-yl]-6H-thieno[2,3-b]pyrrole-5-carboxamide hydrochloride (Intermediate 1) as the amine and N-(tert-butoxycarbonyl)-L-serine as the carboxylic acid.

### Example 28

2-Chloro-N-{(1R,2R)-1-[methyl(L-seryl)amino]-2,3-dihydro-1H-inden-2-yl}-6H-thieno[2,3-b]pyrrole-5-carboxamide hydrochloride

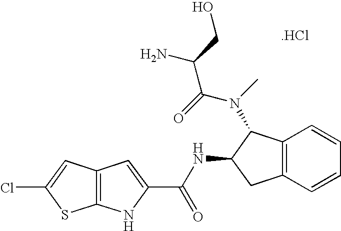

¹H NMR δ: 2.7 (s, 1.5H), 2.9 (s, 1.5H), 3.1 (m, 1H), 3.25 (m, 1H), 3.8 (m, 2H), 4.4 (m, 1H), 5.0 (m, 1H), 5.6 (m, 1H), 5.8 (d, 0.5H), 6.2 (d, 0.5H), 7.25 (m, 6H), 8.1 (m, 3H), 8.7 (d, 0.5H), 9.1 (d, 0.5H), 11.9 (d, 1H); MS m/z 433.

### Example 29

(2S)-N¹-((1R,2R)-2-{[(2-Chloro-6H-thieno[2,3-b]pyrrol-5-yl)carbonyl]amino}-2,3-dihydro-1H-inden-1-yl)-2-hydroxy-N¹-methylpentanediamide

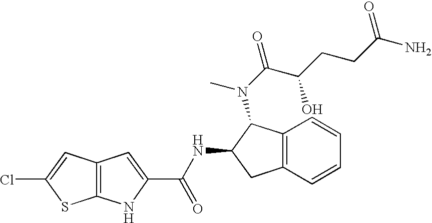

2-Chloro-N-[(1R,2R)-1-(methyl{[(2S)-5-oxotetrahydrofuran-2-yl]carbonyl}amino)-2,3-dihydro-1H-inden-2-yl]-6H-thieno[2,3-b]pyrrole-5-carboxamide (Intermediate 22; 100 mg, 0.22 mmol) was suspended in ammonia (5 mL, 2M in isopropanol, 2.5 mmol) and the mixture heated by microwave irradiation at 150° C. for 30 min. After evaporation of the reaction mixture the crude product by purified by reverse phase preparative HPLC (C18 ODS column, acetonitrile/water gradient 5-95% containing 0.2% TFA eluant) to give the title compound (52 mg, 49%).

¹H NMR δ: 1.65 (m, 1H), 2.0 (m, 1H), 2.2 (m, 2.0H), 2.65 (s, 1.5H), 2.9 (s, 1.5H), 3.0 (m, 1H), 3.3 (m, 1H), 4.35 (m, 1H), 4.7 (d, 0.5H), 4.9 (m, 1H), 5.3 (d, 0.5H), 5.7 (d, 0.5H), 6.2 (d, 0.5H), 6.8 (d, 1H), 7.0 (m, 1.5H), 7.1 (m, 0.5H), 7.2 (s, 1H), 7.35 (m, 4H), 8.65 (m, 1H), 11.9 (s, 1H); MS m/z 475.

The following example was prepared by the method of Example 29 using 2,3-dichloro-N-[(1R,2R)-1-(methyl{[(2S)-5-oxotetrahydrofuran-2-yl]carbonyl}amino)-2,3-dihydro-1H-inden-2-yl]4H-thieno[3,2-b]pyrrole-5-carboxamide (Intermediate 23) as the lactone.

### Example 30

(2S)-N¹-((1R,2R)-2-{[(2,3-Dichloro-4H-thieno[3,2-b]pyrrol-5-yl)carbonyl]amino}-2,3-dihydro-1H-inden-1-yl)-2-hydroxy-N¹-methylpentanediamide

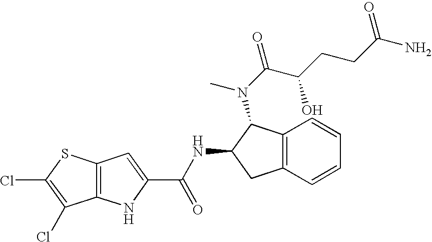

¹H NMR δ: 1.6 (m, 1H), 2.1 (m, 2H), 2.45 (m, 1H), 2.65 (s, 1.5H), 2.85 (s, 1.5H), 3.0 (m, 1H), 3.3 (m, 1H), 4.3 (m, 1H), 4.65 (d, 0.5H), 4.9 (m, 1H), 5.25 (d, 0.5H), 5.7 (d, 0.5H), 6.1 (d, 0.5H), 6.7 (m, 1H), 6.9 (m, 0.5H), 7.1 (m, 1.5H), 7.25 (m, 4H), 8.7 (m, 1H), 12.4 (s, 1H); MS m/z 509.

### Example 31

2-Chloro-N-{(1R,2R)-1-[[(2S)-2-hydroxy-3-methoxypropanoyl](methyl)amino]-2,3-dihydro-1H-inden-2-yl}-6H-thieno[2,3-b]pyrrole-5-carboxamide

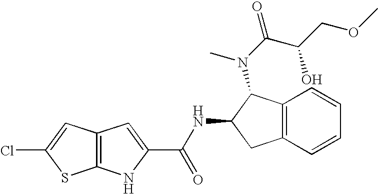

2-Chloro-N-((1R,2R)-1-{methyl[(2S)-oxiran-2-ylcarbonyl]amino}-2,3-dihydro-1H-inden-2-yl)-6H-thieno[2,3-b]pyrrole-5-carboxamide (Intermediate 24; 100 mg, 0.24 mmol) was suspended in sodium methoxide (5 mL, 0.5M solution in methanol) and heated under microwave irradiation at 100° C. for 5 min. Acetic acid (0.5 mL) was then added and the reaction mixture evaporated. The residue was then purified by reverse phase preparative HPLC (C18 ODS column, acetonitrile/water gradient 5-95% containing 0.2% TFA eluant) to give the title compound (33 mg, 32%).

¹H NMR δ: 2.6 (s, 1.5H), 2.8 (s, 1.5H), 3.0 (m, 1H), 3.05 (s, 1.5H), 3.2 (m, 1H), 3.3 (s, 1.5H), 3.45 (m, 2H), 4.5 (t, 1H), 4.85 (m, 1H), 5.7 (d, 0.5H), 6.15 (d, 0.5H), 7.0 (m, 1.5H), 7.1 (m, 0.5H), 7.2 (s, 1H), 7.25 (m, 3H), 8.6 (t, 1H), 11.86 (d, 1H); MS m/z 448.

The following example was prepared by the method of Example 31 using 2,3-dichloro-N-((1R,2R)-1-{methyl[(2S)-oxiran-2-ylcarbonyl]amino}-2,3-dihydro-1H-inden-2-yl)-4H-thieno[3,2-b]pyrrole-5-carboxamide (Intermediate 25) as the oxirane.

### Example 32

2,3-Dichloro-N-{(1R,2R)-1-[[(2S)-2-hydroxy-3-methoxypropanoyl](methyl)amino]-2,3-dihydro-1H-inden-2-yl}-4H-thieno[3,2-b]pyrrole-5-carboxamide

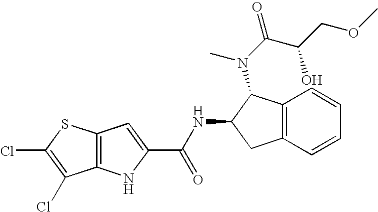

¹H NMR δ: 2.6 (s, 1.5H), 2.8 (s, 1.5H), 3.0 (m, 1H), 3.05 (s, 1.5H), 3.2 (m, 1H), 3.3 (s, 1.5H), 3.45 (m, 2H), 4.55 (t, 1H), 4.9 (m, 1H), 5.75 (d, 0.5H), 6.2 (d, 0.5H), 6.95 (m, 0.5H), 7.1 (m, 1.5H), 7.3 (m, 3H), 8.7 (t, 1H), 12.42 (d, 1H); MS m/z 482.

### Example 33

2,3-Dichloro-N-{(1R,2R)-1-[[(2R)-2-hydroxy-3-(methylsulfonyl)propanoyl](methyl)amino]-2,3-dihydro-1H-inden-2-yl}-4H-thieno[3,2-b]pyrrole-5-carboxamide

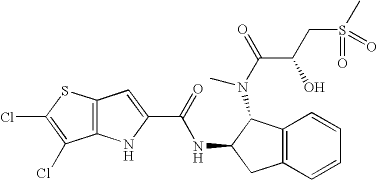

2,3-Dichloro-N-{(1R,2R)-1-[[(2R)-2-hydroxy-3-(methylthio)propanoyl](methyl)amino]-2,3-dihydro-1H-inden-2-yl}-4H-thieno[3,2-b]pyrrole-5-carboxamide (Example 22; 200 mg, 0.40 mmol) was dissolved in DMF (5 mL) at 0° C., and m-CPBA (218 mg, 70% strength, 0.88 mmol) was added. The reaction was stirred for 3 h at ambient temperature before purification by reverse phase HPLC (C18 ODS column, 5-95% acetonitrile/water gradient containing 0.2% TFA) to give the title compound (150 mg, 70%) as a white solid.

¹H NMR δ: 2.80 (d, 3H), 2.89 (d, 3H), 2.97 (m, 1H), 3.22 (m, 1H), 3.41 (dd, 2H), 4.88 (m, H), 5.90 (dd, 1H), 7.18 (m, 5H), 8.70 (dd, 1H), 12.39 (d, 1H); MS m/z 530.1.

### Example 34

N-{(1R,2R)-1-[[(2S)-3-Amino-2-hydroxypropanoyl](methyl)amino]-2,3-dihydro-1H-inden-2-yl}-2,3-dichloro-4H-thieno[3,2-b]pyrrole-5-carboxamide hydrochloride

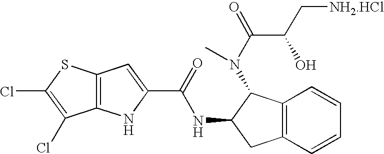

tert-Butyl {(2S)-3-[((1R,2R)-2-{[(2,3-dichloro-4H-thieno[3,2-b]pyrrol-5-yl)carbonyl]amino}2,3-dihydro-1H-inden-1-yl)(methyl)amino]-2-hydroxy-3-oxopropyl}carbamate (Intermediate 30; 291 mg, 0.51 mmol) was dissolved in DCM (15 mL), 4M HCl/dioxane (15 mL) was added and the reaction mixture stirred for 1 h. The solvent was evaporated, the residue slurried in ether (30 mL) and filtered to give the title compound (190 mg, 74%) as a white solid.

¹H NMR δ: 2.77 (d, 3H), 3.03 (m, 2H), 3.25 (m, 1H), 4.76 (m, 2H), 5.79 (d, 1H), 6.08 (d, 1H), 7.18 (m, 5H), 7.90 (s, 3H), 8.90 (dd, 1H), 12.48 (d, 1H); MS m/z 466.9.

### Example 35

(2S)-N¹-((1R,2R)-2-{[(2,3-Dichloro-4H-thieno[3,2-b]pyrrol-5-yl)carbonyl]amino}-2,3-dihydro-1H-inden-1-yl)-2-hydroxy-N¹,N⁴-dimethylsuccinamide

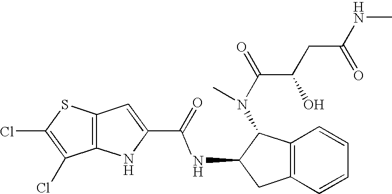

Methylamine (11 mL, 2M in THF, 22 mmol), HOBT (1.49 g, 11 mmol), [(4S)-2,2-dimethyl-5-oxo-1,3-dioxolan-4-yl]acetic acid (1.74 g, 10 mmol) and EDAC (2.11 g, 11 mmol) were dissolved in DCM (50 mL). The reaction was stirred at ambient temperature for approximately 16 h before evaporation and purification by flash column chromatography on silica gel eluting with 6-10% MeOH/DCM. The resulting gum was dissolved in 4M HCl/dioxane (30 ml) before adding water (1.5 ml) and stirring for 1 h. The solvent was evaporated to afford the crude acid. This acid (448 mg, 3.0 mmol), 2,3-dichloro-N-[(1R,2R)-1-(methylamino)-2,3-dihydro-1H-inden-2-yl]4,6a-dihydro-3aH-thieno[3,2-b]pyrrole-5-carboxamide hydrochloride (Intermediate 16; 423 mg, 1.0 mmol), HOBT (205 mg, 1.5 mmol), and triethylamine (0.62 ml, 4.5 mmol) were suspended in DMF (15 mL) and stirred at room temperature. EDCI (292 mg, 1.5 mmol) was added and stirring was continued for a further 18 hours. The reaction mixture was purified by reverse phase HPLC (C18 ODS column 5-95% acetonitrile/water gradient containing 0.2% TFA) to give the title compound (30 mg, 6%) as a white solid.

¹H NMR δ: 2.71 (m, 9H), 3.50 (m, 1H), 4.91 (m, 2H), 5.95 (dd, 1H), 6.85 (m, 2H), 7.24 (m, 5H), 8.10 (d, 1H), 10.36 (d, 1H); MS m/z 508.9.

The following example was made by the process of Example 35 using dimethylamine as the amine.

### Example 36

(2S)-N¹-((1R,2R)-2-{[(2,3-Dichloro-4H-thieno[3,2-b]pyrrol-5-yl)carbonyl]amino}-2,3-dihydro-1H-inden-1-yl)-2-hydroxy-N¹,N⁴,N⁴-trimethylsuccinamide

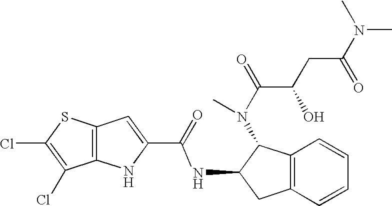

¹H NMR δ: 2.93 (m, 9H), 3.49 (s, 3H), 3.58 (m, 1H), 4.99 (m, 2H), 6.03 (dd, 1H), 7.00 (m, 7H), 8.39 (d, 1H), 10.17 (s, 1H); MS m/z 522.9.

### Example 37

2-Chloro-N-{(1R,2R)-1-[glyceroyl(2-hydroxyethyl)amino]-2,3-dihydro-1H-inden-2-yl}-6H-thieno[2,3-b]pyrrole-5-carboxamide

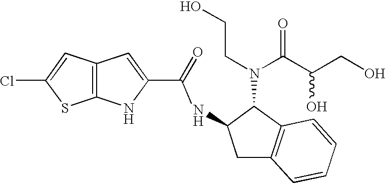

2-Chloro-N-((1R,2R)-1-{[(2,2-dimethyl-1,3-dioxolan-4-yl)carbonyl][2-(tetrahydro-2H-pyran-2-yloxy)ethyl]amino}-2,3-dihydro-1H-inden-2-yl)-6H-thieno[2,3-b]pyrrole-5-carboxamide (Intermediate 26; 250 mg, 0.472 mmol) was dissolved in acetic acid (6 mL, 20% aqueous) and warmed to 60° C. for 4 h. The reaction was cooled and the volatiles removed under reduced pressure. The residue was dissolved in EtOAc (15 mL), washed with saturated NaHCO₃ (5 mL), water (5 mL) and brine (5 mL), dried (MgSO₄) and the volatiles removed under reduced pressure. The residue was purified by flash column chromatography (EtOAc) to give the title compound (110 mg, 56%) as a foam.

¹H NMR (mixture of rotamers and diastereomers) δ: 2.8 (m, 2H), 3.4 (m, 6H), 4.8 (m, 5H), 5.68 (d, 0.5H), 5.8 (d, 0.5H), 7.2 (m, 6H), 8.55 (m, 1H), 11.9 (s, 1H); MS m/z 462, 464 (M−H).

The following examples were made by the process of Example 37, using 2-Chloro-N-((1R,2R)-1-{[((R)-2,2-dimethyl-1,3-dioxolan-4-yl)carbonyl][2-(tetrahydro-2H-pyran-2-yloxy)ethyl]amino}-2,3-dihydro-1H-inden-2-yl)-6H-thieno[2,3-b]pyrrole-5-carboxamide (Intermediate 27) or 2-chloro-N-((1R,2R)-1-[{((S)-2,2-dimethyl-1,3-dioxolan-4-yl)carbonyl][2-(tetrahydro-2H-pyran-2-yloxy)ethyl]amino}-2,3-dihydro-1H-inden-2-yl)-6H-thieno[2,3-b]pyrrole-5-carboxamide (Intermediate 28) as the protected triol.

### Example 38

2-Chloro-N-{(1R,2R)-1-[[(2R)-2,3-dihydroxypropanoyl](2-hydroxyethyl)amino]-2,3-dihydro-1H-inden-2-yl}-6H-thieno[2,3-b]pyrrole-5-carboxamide

### Example 39

2-Chloro-N-{(1R,2R)-1-[[(2S)-2,3-dihydroxypropanoyl](2-hydroxyethyl)amino]-2,3-dihydro-1H-inden-2-yl}-6H-thieno[2,3-b]pyrrole-5-carboxamide

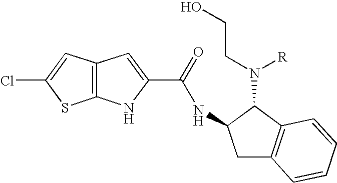

| Example | R | ¹H NMR δ | M/z |
|---|---|---|---|
| 38 | 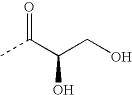 | 2.8(m, 2H), 3.4(m, 6H), 4.8(m, 5H), 5.68(d, 1H), 7.2(m, 6H), 8.55(m, 1H), 11.9(s, 1H) | 462, 464 (M − H) |
| 39 | 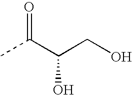 | 2.8(m, 2H), 3.4(m, 6H), 4.8(m, 5H), 5.8(d, 1H), 7.2(m, 6H), 8.55(m, 1H), 11.9(s, 1H) | 462, 464 (M − H) |

Intermediates:
Intermediate 1: 2-Chloro-N-[(1R,2R)-1-(methylamino)-2,3-dihydro-1H-inden-2-yl]-6H-thieno[2,3-b]pyrrole-5-carboxamide hydrochloride salt

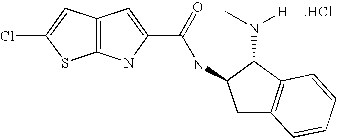

tert-Butyl ((1R,2R)-2-{[(2-chloro-6H-thieno[2,3-b]pyrrol-5-yl)carbonyl]amino}-2,3-dihydro-1H-inden-1-yl)methylcarbamate (Intermediate 2; 5.8 g, 13.0 mmol) was dissolved in HCL solution (4N in dioxane, 40 ml) and stirred at ambient temperature for 24 hours. The volatiles were removed by evaporation under reduced pressure and the residue dried in vacuo to give the title compound (3.0 g, 60%) as a powder.

¹H NMR δ: 2.7 (s, 3H), 3.05 (dd, 1H), 3.5 (dd, 1H), 4.82 (m, 2H), 7.09 (s, 1H), 7.15 (s, 1H), 7.35 (m, 3H), 7.78 (d, 1H), 8.9 (d, 1H), 9.55 (broad d, 2H), 11.95 (s, 1H); MS m/z 346, 348.
Intermediate 2: tert-Butyl ((1R,2R)-2-{[(2-chloro-6H-thieno[2,3-b]pyrrol-5-yl)carbonyl]amino}-2,3-dihydro-1H-inden-1-yl)methylcarbamate

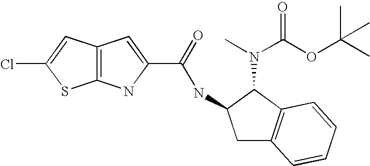

2-Chloro-6H-thieno[2,3-b]pyrrole-5-carboxylic acid (Intermediate 3; 3.5 g, 17.16 mmol), tert-butyl [(1R,2R)-2-amino-2,3-dihydro-H-inden-1-yl]methylcarbamate (Intermediate 5; 4.5 g, 17.16 mmol), DIPEA (2.9 ml, 17.16 mmol) and HOBT (2.32 g, 17.16 mmol) were dissolved in DCM (60 ml), stirred for 5 minutes, EDCI (4.11 g, 21.45 mmol) added and the mixture stirred at ambient temperature for 24 hours. The volatiles were removed by evaporation under reduced pressure and EtOAc (200 mL) added. The organic phase was washed with water (100 mL), brine (50 mL) and dried (MgSO₄), filtered and the solvent removed under reduced pressure. The residue was purified by column chromatography (SiO₂, EtOAc:Hexane) to afford the title compound (5.8 g, 76%) as an oil.

¹H NMR δ: 1.3 (s, 9H), 2.63 (s, 3H), 3.1 (m, 2H), 4.75 (m, 1H), 5.63 (m, 1H), 7.0 (m, 2H), 7.16 (s, 1H), 7.25 (m, 3H), 8.48 (m, 1H), 11.83 (s, 1H).
Intermediate 3: 2-Chloro-6H-thieno[2,3-b]pyrrole-5-carboxylic acid

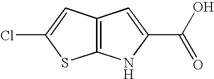

Sodium hydroxide (2N, 15 mL) was added to a methanol (50 mL) solution of 2-chloro-5-methoxycarbonyl-6H-thieno[2,3-b]pyrrole (Intermediate 4, 777 mg, 3.6 mmol) and the mixture heated at reflux for 5 hours. The reaction was cooled to ambient temperature, water (250 mL) added and the aqueous phase was washed with Et₂O (2×50 mL), acidified to pH 2 with HCl (2N) and extracted with EtOAc (3×50 mL). The combined organic phases were washed with water (2×50 mL), brine (50 mL), dried (MgSO₄) and the solvent removed under reduced pressure to afford the title compound (705 mg, 97%) as a pale pink solid.

¹H NMR (CDCl₃)δ: 12.6-12.7 (1H, b), 12.0-12.1 (1H, b), 7.15 (1H, s), 6.9 (1H, s); m/z 183, 185.
Intermediate 4: 2-Chloro-5-methoxycarbonyl-6H-thieno[2,3-b]pyrrole

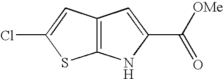

Sodium (659 mg, 28.7 mL) was added to dry methanol (20 mL) and the mixture stirred at ambient temperature for 30 mins before cooling to −20° C. 2-Chlorothiophene-3-carboxaldehyde (Gronowitz et al,. Tetrahedron Vol. 32 1976 p. 1403; 1.17 g, 7.2 mmol) and methyl azidoacetate (3.3 g, 28.7 mmol) were added as a methanol (10 mL) solution and the reaction was stirred from −20° C. to 10° C. over 16 hours. The reaction was poured on saturated NH₄Cl (300 mL) and extracted with DCM (3×100 mL). The combined organic phases were washed with water (2×100 mL), brine (100 mL), dried (MgSO₄) and the solvent removed under reduced pressure. The crude product was redissolved in xylene (50 mL) and added dropwise to refluxing xylene (150 mL) and stirred for at reflux for a further 30 mins after the addition was complete. The solvent was removed under reduced pressure to afford a yellow solid which was recrystallised (25:75, EtOAC:isohexane) to afford the title compound (1.06 g, 69%) as a solid.

¹H NMR (CDCl₃) δ: 9.4-9.2 (1H, br), 7.0 (1H, s), 6.9 (1H, s), 3.9 (3H, s); m/z 214, 216.
Intermediate 5: tert-Butyl [(1R,2R)-2-amino-2,3-dihydro-1H-inden-1-yl]methyl carbamate

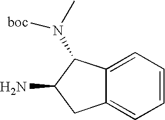

(1R,2S)-1-[(tert-Butoxycarbonyl)(methyl)amino]-2,3-dihydro-1H-inden-2-yl methanesulfonate (Intermediate 6; 3.0 g, 8.8 mmol) and sodium azide (2.3 g, 35.2 mmol) in dry DMA (30 mL) was heated to 90° C. for 7 hours. The reaction was cooled and ethyl acetate (100 mL) added. The mixture was washed with water (6×25 mL), brine (50 mL) and dried (MgSO₄). 10% Palladium on carbon (400 mg) was added to the organic solution which was stirred under a hydrogen atmosphere for 4 h, filtered through Celite and evaporated. The residue was purified by column chromatography (EtOAc and then DCM:MeOH 9:1) to afford the title compound (1.2 g, 55%) as a pale brown oil.

¹H NMR δ: 1.45 (m, 9H), 2.6 (s, 3H), 2.8 (m, 1H), 3.3 (m, 1H), 4.45 (m, 1H), 5.55 (dd, 1H), 7.26 (m, 4H); MS m/z 264.
Intermediate 6: (1R,2S)-1-[(tert-Butoxycarbonyl)(methyl)amino]-2,3-dihydro-1H-inden-2-yl methanesulfonate

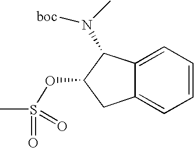

tert-Butyl [(1R,2S)-2-hydroxy-2,3-dihydro-1H-inden-1-yl]methylcarbamate (Intermediate 7; 3.0 g, 11.4 mmol) was dissolved in dry THF (40 mL) at 10° C. A solution of methane sulphonyl chloride (1.44 g, 12.55 mmol) in dry THF (10 mL) was added, the reaction allowed to warm to ambient temperature and stirred for 30 mins. The volatiles were removed by evaporation under reduced pressure and ethyl acetate (100 mL) added. The mixture was washed with water (2×50 mL), brine (50 mL) and the organic phase was dried (MgSO₄), filtered and evaporated. The residue was purified by column chromatography (EtOAc:Hexane) to afford the title compound (3.1 g, 80%) as a colourless syrup.

¹H NMR δ: 1.46 (s, 9H), 2.61 (s, 3H), 3.12 (m, 1H), 3.18 (s, 3H), 3.32 (m, 1H), 5.45 (m, 1H), 5.68 (m, 1H), 7.28 (m, 4H); MS m/z 342.
Intermediate 7: tert-Butyl [(1R,2S)-2-hydroxy-2,3-dihydro-1H-inden-1-yl]methylcarbamate

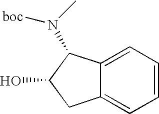

tert-Butyl methyl[(1R,2S)-2-(tetrahydro-2H-pyran-2-yloxy)-2,3-dihydro-1H-inden-1-yl]carbamate (Intermediate 8; 4.0 g, 11.5 mmol) was dissolved in methanol (50 mL), 4-toluene sulphonic acid added and the reaction stirred at ambient temperature for 2 hours. Saturated NaHCO₃ (50 mL), water (100 mL) was added and ethyl acetate (100 mL) was added and the mixture stirred for 30 mins. The organic phase was separated, washed with water (50 mL), brine (50 mL) and dried (MgSO₄). The volatiles were removed by evaporation under reduced pressure to give the title compound (3.0 g, 99%) as an oil.

¹H NMR δ: 1.45 (s, 9H), 2.6 (s, 3H), 2.75 (m, 1H), 3.05 (m, 1H), 4.5 (m, 1H), 5.05 (m, 1H), 5.34 (m, 1H), 7.03-7.3 (m, 4H).
Intermediate 8: tert-Butyl methyl[(1R,2S)-2-(tetrahydro-2H-pyran-2-yloxy)-2,3-dihydro-1H-inden-1-yl]carbamate

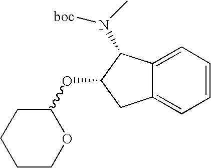

tert-Butyl [(1R,2S)-2-(tetrahydro-2H-pyran-2-yloxy)-2,3-dihydro-1H-inden-1-yl]carbamate (Intermediate 9; 4.0 g, 12.0 mmol) was dissolved in dry DMA (25 mL) at 5° C. 60% Sodium hydride (575 mg, 14.4 mmol) was added, the reaction stirred at 5° C. for 30 mins, allowed to warm to ambient temperature and stirred for a further 30 mins. Methyl iodide (896 μL, 14.4 mmol) was added and the reaction stirred at ambient temperature for 3 hours. The reaction was poured into water (100 mL) and extracted with ethyl acetate (2×50 ml). The organic extracts were washed with water (6×25 mL), brine (50 mL) and dried (MgSO₄). The volatiles were removed by evaporation under reduced pressure to give the title compound (4.1 g, 97%) as an oil.

¹H NMR δ: 1.4-1.9 (m, 6H), 1.5 (s, 9H), 2.7 (dd, 3H), 2.85-3.3 (m, 2H), 3.5 (m, 1H), 3.7-4.0 (m, 1H), 4.6-4.9 (m, 2H), 5.5-5.85 (m, 1H), 7.2 (s, 4H).
Intermediate 9: tert-Butyl [(1R,2S)-2-(tetrahydro-2H-pyran-2-yloxy)-2,3-dihydro-1H-inden-1-yl]carbamate

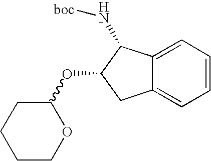

tert-Butyl [(1R,2S)-2-hydroxy-2,3-dihydro-1H-inden-1-yl]carbamate (Intermediate 10, 7.0 g, 28.1 mmol) and 3,4-dihydro-2H-pyran (4.7 g, 56.2 mmol) dissolved in DCM (50 mL). 4-Toluenesulphonic acid pyridinium salt (100 mg) was added and the reaction stirred for 4 hours at ambient temperature. The reaction was diluted with ethyl acetate (100 mL), washed with water (2×50 mL), brine (50 mL) and dried (MgSO₄). The volatiles were removed by evaporation under reduced pressure to give the title compound (8.9 g, 95%) as an oil.

¹H NMR δ: 1.25-1.85 (m, 6H), 1.45 (s, 9H), 2.85-3.1 (m, 2H), 3.4 (m, 1H), 3.8 (m, 1H), 4.35-5.1 (m, 3H), 6.8 (dd, 1H), 7.2(s, 1H).
Intermediate 10: tert-Butyl [(1R,2S)-2-hvdroxy-2,3-dihydro-1H-inden-1-yl]carbamate

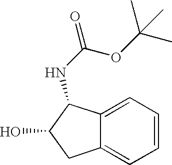

(1R,2S)-1-Amino-2,3-dihydro-1H-inden-2-ol (CAS Reg. No. 136030-00-7; 10 g, 67.1 mmol) was dissolved in DCM (550 mL) and Et₃N (18.7 mL, 134.2 mmol). Di-tert-butyl dicarbonate (18.3 g, 83.9 mmol) in DCM (50 mL) was added and the mixture stirred at ambient temperature for 20 hours, and then evaporated. EtOAc (200 mL) was added, the solution washed with water (200 mL), dried (MgSO₄) and the volatiles removed under reduced pressure. The crude product was purified by flash column chromatography (SiO₂, 4:1, iso-hexane:EtOAc eluent) to provide the title compound (16.1 g, 96%) as a white solid.

¹H NMR δ: 1.42 (m, 9H), 2.78 (dd, 1H), 3.00 (dd, 1H), 4.36 (m, 1H), 4.84 (m, 1H), 4.95 (m, 1H), 6.3 (d, 1H), 7.13 (m, 4H).
Intermediate 11: Ethyl N-[(acetyloxy)acetyl]-N-((1R,2R)-2-{[(2-chloro-6H-thieno[2,3-b]pyrrol-5-yl)carbonyl]amino}-2,3-dihydro-1H-inden-1-yl)glycinate

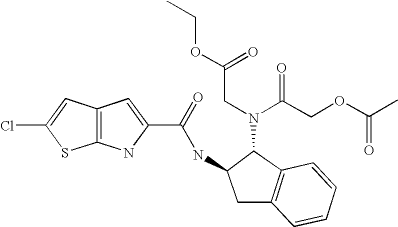

Ethyl N-((1R,2R)-2-{[(2-chloro-6H-thieno[2,3-b]pyrrol-5-yl)carbonyl]amino}-2,3-dihydro-1H-inden-1-yl)glycinate (Intermediate 12; 820 mg, 2.0 mmol), acetoxyacetic acid (236 mg, 2.0 mmol) and HOBT (270 mg, 2.0 mmol) were dissolved in DCM (20 ml), stirred for 5 minutes, EDCI (480 mg, 2.5 mmol) added and the mixture stirred at ambient temperature for 4 hours. The volatiles were removed by evaporation under reduced pressure and EtOAc (25 mL) added. The mixture was washed with water (2×10 mL), brine (10 mL) and dried (MgSO₄), filtered and solvent removed under reduced pressure. The residue was purified by column chromatography (SiO₂, EtOAc:Hexane) to afford the title compound (750 mg, 75%) as a foam.

¹H NMR δ: 1.1 (t, 3H), 2.02 (s, 1.5H), 2.08 (s, 1.5H), 2.9 (m, 1H), 3.25 (m, 1H), 3.68 (m, 1H), 4.0 (m, 3H), 4.68 (m, 1H), 5.0 (m, 2H), 5.45 (d, 0.7H), 5.9 (d, 0.3H), 7.2 (m, 6H), 8.45 (d, 0.3H), 8.55 (d, 0.7H), 11.8 (s, 0.3H), 11.84 (s, 0.7H).
Intermediate 12: Ethyl N-((1R,2R)-2-{[(2-chloro-6H-thieno[2,3-b]pyrrol-5-yl)carbonyl]amino}-2,3-dihydro-1H-inden-1-yl)glycinate

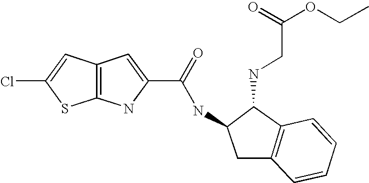

N-[(1R,2R)-1-Amino-2,3-dihydro-1H-inden-2-yl]-2-chloro-6H-thieno[2,3-b]pyrrole-5-carboxamide hydrochloride salt (Intermediate 13, 1.8 g, 5.0 mmol), DIPEA (856 μl, 5.0 mmol), ethyl glyoxalate (1.02 ml, 50% solution in toluene, 5.0 mmol) and acetic acid (300 μl, 5.0 mmol) were dissolved in anhydrous THF (40 ml) and stirred at ambient temperature for 1 hour. Sodium triacetoxy borohydride (1.6 g, 7.5 mmol) was added, the reaction stirred at ambient temperature for 4 hours. HCl (1N, 10 ml) and EtOAc (75 ml) were added and the phases separated. The organic phase was washed with saturated NaHCO₃ (25 ml), water (25 ml), brine (25 ml), dried (MgSO₄) filtered and the solvent removed under reduced pressure. The residue was purified by column chromatography (SiO₂; EtOAc:Hexane) to afford the title compound (1.7 g, 81%) as a solid.

¹H NMR δ: 1.27 (t, 3H), 1.80 (br s, 1H), 2.82 (dd, 1H), 3.7 (m, 3H), 4.22 (m, 3H), 4.5 (m, 1H), 6.78 (m, 2H0, 6.85 (s, 1H), 7.25 (m, 3H), 7.37 (m, 1H), 10.68 (s, 1H).
Intermediate 13: N-[(1R,2R)-1-Amino-2,3-dihydro-1H-inden-2-yl]-2-chloro-6H-thieno[2,3-b]pyrrole-5-carboxamide hydrochloride salt

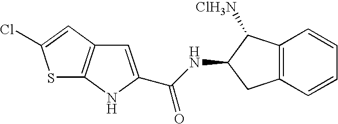

tert-Butyl ((1R,2R)-2-{[(2-chloro-6H-thieno[2,3-b]pyrrol-5-yl)carbonyl]amino}-2,3-dihydro-1H-inden-1-yl)carbamate (Intermediate 14; 3.5 g, 8.1 mmol) was suspended in DCM (10 mL). HCl (20 mL, 4N in dioxane, 80 mmol) was added and reaction stirred at ambient temperature for 48 h. The volatiles were removed under reduced pressure; the resulting solid was azeotroped with EtOAc (4×50 mL) and dried in vacuo to afford the title compound (2.91 g, 97%) as a brown solid.

¹H NMR δ: 3.04 (dd, 1H), 3.87 (dd, 1H), 4.65 (m, 1H), 4.83 (br s, 1H), 7.15 (m, 2H), 7.33 (m, 3H), 7.69 (d, 1H), 8.80 (br s, 3H), 8.96 (d, 1H), 12.00 (s, 1H). m/z 330, 332 [M−H]⁻.
Intermediate 14: tert-Butyl ((1R,2R)-2-{[(2-chloro-6H-thieno[2,3-b]pyrrol-5-yl)carbonyl]amino}-2,3-dihydro-1H-inden-1-yl)carbamate

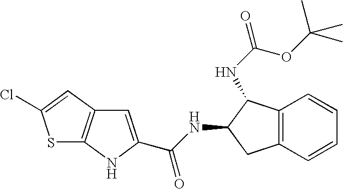

(1R, 2R)-2-Amino-1-[(1,1-dimethylethoxy)carbonylamino]indan (Intermediate 15; 3.7 g, 15 mmol), 2-chloro-6H-thieno[2,3-b]pyrrole-5-carboxylic acid (Intermediate 3; 3.0 g, 15 mmol), HOBT (2.03 g, 15 mmol) and DIPEA (2.83 mL, 16.5 mmol) were suspended in DCM (60 mL) and stirred at ambient temperature for 5 mins. EDAC (3.57 g, 18.75 mmol) was added and the mixture stirred at ambient temperature for 20 hours. The resulting precipitate was filtered, washed with DCM (2×20 mL) and dried to afford the title compound (5.35 g, 67%) as a yellow solid.

¹H NMR δ: 1.38 (s, 9H), 2.81 (dd, 1H), 3.17 (dd, 1H), 4.56 (m, 1H), 5.14 (m, 1H), 7.01 (s, 1H), 7.16 (m, 5H), 7.32 (d, 1H), 8.47 (d, 1H), 11.82 (s, 1H).
Intermediate 15: (1R,2R)-2-Amino-1-[(1,1-dimethylethoxy)carbonylamino]indan

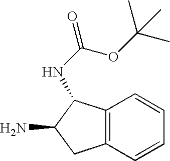

Cis-1-[(1,1-Dimethylethoxy)carbonylamino]-2-hydroxyindan (Intermediate 10; 14.0 g, 56.2 mmol) was dissolved in DCM (200 ml) and triethylamine (11.8 ml, 84.3 mmol). Methanesulfonyl chloride (7.1 g, 61.9 mmol) dissolved in DCM (20 ml) was added and the mixture stirred at room temperature for 3 hours. The mixture was evaporated and EtOAc (250 ml) added. After washing with water and drying over magnesium sulphate the organic solution was evaporated to yield cis-1-[(1,1-dimethylethoxy)carbonylamino]-2-methanesulphonyloxyindan (9.7 g, 98%) as a white solid.

¹H NMR : 1.45 (s, 9H), 3.15 (m, 2H), 3.18 (s, 3H), 5.20 (m, 1H), 5.35 (m, 1H), 7.15 (m, 4H), 7.45 (d, 1H).

Cis-1-[(1,1-dimethylethoxy)carbonylamino]-2-methanesulphonyloxyindan (18.1 g, 55.3 mmol) was dissolved in dry dimethyl acetamide (100 ml). Sodium azide (5.4 g, 83.0 mmol) was added and the mixture heated to 90° C. for 6 hours. The reaction was cooled, diluted with ethyl acetate (150 ml), washed with water (6×200 ml) and dried over magnesium sulphate. 10% Palladium on activated carbon was added and the mixture stirred under a hydrogen atmosphere for 24 hours. Filtration through celite followed by evaporation gave the title compound (2.6 g, 98%) as a white solid.

¹H NMR: 1.45 (s, 9H), 2.50 (dd, 1H), 3.05 (dd, 1H), 3.30 (m, 3H), 4.55 (m, 1H), 7.1 (m, 5H).
Intermediate 16: 2,3-Dichloro-N-[(1R,2R)-1-(methylamino)-2,3-dihydro-1H-inden-2-yl]-4,6a-dihydro-3aH-thieno[3,2-b]pyrrole-5-carboxamide hydrochloride

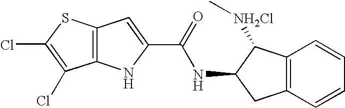

This intermediate was prepared by the method of Intermediate 1, using: tert-butyl ((1R,2R)-2-{[(2,3-dichloro-4,6a-dihydro-3aH-thieno[3,2-b]pyrrol-5-yl)carbonyl]amino}-2,3-dihydro-1H-inden-1-yl)methylcarbamate (Intermediate 17) as the carbamate.

¹H NMR δ: 2.69 (m, 3H), 3.04 (dd, 1H), 3.51 (m, 1H), 4.85 (m, 2H), 7.19 (s, 2H), 7.35 (m, 3H), 7.75 (d, 1H), 9.10 (d, 1H), 9.55 (d, 2H), 12.58 (s, 1H); MS m/z 378.2.
Intermediate 17: tert-butyl ((1R,2R)-2-{[(2,3-dichloro-4,6a-dihydro-3aH-thieno[3,2-b]pyrrol-5-yl)carbonyl]amino}-2,3-dihydro-1H-inden-1-yl)methylcarbamate

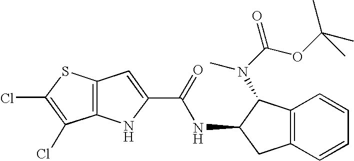

This intermediate was made by the process of Intermediate 2, using tert-Butyl [(1R,2R)-2-amino-2,3-dihydro-1H-inden-1-yl]methylcarbamate (Intermediate 5) as the amine and 5-Carboxy-2,3-dichloro-4H-thieno[3,2-b]pyrrole (Intermediate 18) as the carboxylic acid.

¹H NMR δ: 1.28 (d, 9H), 2.65 (s, 3H), 3.09 (m, 2H), 4.79 (m, 1H), 5.62 (dd, 1H), 7.05 (m, 2H), 7.24 (m, 3H), 8.59 (m, 1H), 12.42 (d, 1H); MS m/z 478.2.

Intermediate 18: 5-Carboxy-2,3-dichloro-4H-thieno[3,2-b]pyrrole

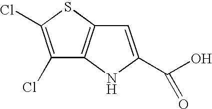

2,3-Dichloro-5-methoxycarbonyl-4H-thieno[3,2-b]pyrrole (Intermediate 19; 1.22 g, 5.66 mmol) was taken up in MeOH (10 mL) and was heated under reflux. Aqueous lithium hydroxide (3.0 mL, 2.0 M, 6.0 mmol) was added portionwise over 45 mins. The mixture was heated at reflux for 1 h before being cooled and concentrated. Water (15 mL) was added and the solution was neutralised using 2 M aqueous HCl (5.66 mL). The solution was extracted using EtOAc (3×20 mL), and the combined organic layers were concentrated to afford the title compound (1.18 g, 100%) as a yellow solid

¹H NMR (CDCl₃) δ: 7.0 (1H, s); MS m/z 234.
Intermediate 19: 2,3-Dichloro-5-methoxycarbonyl-4H-thieno[3,2-b]pyrrole

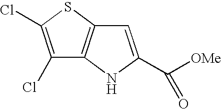

Sodium methoxide solution (5 mL, 28% w/v solution, 25.9 mmol) was diluted with MeOH (10 mL) and was cooled to −25° C. under nitrogen. A solution of 3,4-dichloro-5-carbonyl-4H-thieno[3,2,b]pyrrole (DE 2814798; 1.2 g. 6.63 mmol) and methyl azidoacetate (3.0 g, 26.09 mmol) in MeOH (15 mL) was added dropwise, maintaining the temperature at −25° C. On completion of addition the solution was allowed to warm to 5° C. over a period of approximately 16 h. The solution was added to saturated aqueous ammonium chloride (250 mL) and the mixture was extracted using DCM. The combined organic layers were concentrated at ambient temperature. The residue was taken up in xylene (30 mL) and this solution was added dropwise to xylene (120 mL) under reflux. The solution was heated under reflux for 30 mins before being cooled and concentrated. The title compound was purified by crystallisation (EtOAc/isohexane) to afford the title compound (1.08 g, 65%) as a solid.

¹H NMR (CDCl₃) δ: 9.2 (1H, br), 7.0 (1H, s), 3.9 (3H, s); MS m/z 248.
Intermediate 20: 2,3-Dichloro-N-{(1R,2R)-1-[{[(4R)-2,2-dimethyl-1,3-dioxolan-4-yl]carbonyl}(methyl)amino]-2,3-dihydro-1H-inden-2-yl}-4H-thieno[3,2-b]pyrrole-5-carboxamide

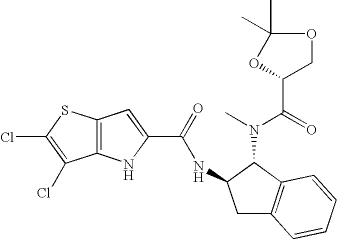

2,3-Dichloro-N-[(1R,2R)-1-(methylamino)-2,3-dihydro-1H-inden-2-yl]-4H-thieno[3,2-b]pyrrole-5-carboxamide hydrochloride (Intermediate 16; 320 mg, 0.768 mmol), (4R)-2,2-dimethyl-1,3-dioxolane-4-carboxylic acid potassium salt (212 mg, 1.15 mmol) and DIPEA (131 μL, 0.768 mmol) in anhydrous DMA (5 ml) was added HOBT (104 mg, 0.768 mmol) and EDCI (184 mg, 0.96 mmol). The reaction was stirred at ambient temperature for 1.5 h, water (25 ml) added and the mixture filtered. The solid was washed with water (2×20 ml), dried in vacuo and purified by flash chromatography (EtOAc:hexane 1:1) to give the title compound (285 mg, 73%) as a powder.

¹H NMR (mixture of rotamers) δ: 1.2 (m, 6H), 2.65 (s, 1.5H), 2.85 (m, 1.5H), 3.05 (m, 1H), 3.2 (m, 1H), 4.1 (m, 2H), 4.9 (m, 2H), 5.55 (d, 0.5H), 6.1 (d, 0.5H), 7.1 (m, 2H), 7.25 (m, 3H), 8.5 (d, 0.5H), 8.6 (d, 0.5H), 12.4 (m, 1H); MS m/z 506, 508 (M−H).

The following intermediate was made by the process of Intermediate 20, using 2,3-dichloro-N-[(1R,2R)-1-(methylamino)-2,3-dihydro-1H-inden-2-yl]-4H-thieno[3,2-b]pyrrole-5-carboxamide hydrochloride (Intermediate 16) as the amine and (4S)-2,2-dimethyl-1,3-dioxolane-4-carboxylic acid potassium salt as the carboxylate source.
Intermediate 21: 2,3-Dichloro-N-{(1R,2R)-1-[{[(4S)-2,2-dimethyl-1,3-dioxolan-4-yl]carbonyl}(methyl)amino]-2,3-dihydro-1H-inden-2-yl}-4H-thieno[3,2-b]pyrrole-5-carboxamide

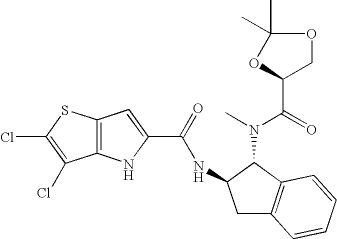

¹H NMR (mixture of rotamers): 1.2 (m, 6H), 2.65 (s, 1.5H), 2.85 (s, 1.5H), 3.0 (m, 1H), 3.25 (m, 1H), 4.1 (m, 1H), 4.25 (m, 1H), 4.9 (m, 2H), 5.73 (d, 0.5H), 6.1 (d, 0.5H), 7.1 m, 2H), 7.25 (m, 3H), 8.62 (d, 0.5H), 8.75 (d, 0.5H), 12.42 (m, 1H); MS m/z 506, 508 (M−H).
Intermediate 22: 2-Chloro-N-[(1R,2R)-1-(methyl{[(2S)-5-oxotetrahydrofuran-2-yl]carbonyl}amino)-2,3-dihydro-1H-inden-2-yl]-6H-thieno[2,3-b]pyrrole-5-carboxamide

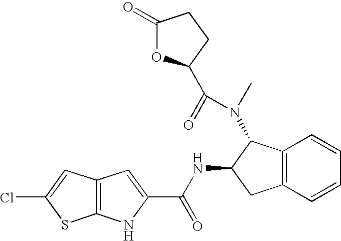

DIPEA (173 μL, 1.0 mmol), (2S)-5-oxotetrahydrofuran-2-carboxylic acid (260 mg, 2 mmol) and EDAC (328 mg, 2.0 mmol) were added to a suspension of 2-chloro-N-[(1R,2R)-1-(methylamino)-2,3-dihydro-1H-inden-2-yl]-6H-thieno[2,3-b]pyrrole-5-carboxamide hydrochloride (intermediate 1; 381.5 mg, 1.0 mmol) in anhydrous DMF (5 mL). The reaction was stirred at ambient temperature for 3 h. and then diluted with EtOAc (50 mL). The solution was washed with water (4×20 mL), dried (MgSO₄) and evaporated to give a gum which was triturated with ether to give the title compound (330 mg, 72%) as a white solid.

¹H NMR δ: 2.2 (m, 2H), 2.5 (, 2H), 2.7 (s, 1.5H), 2.8 (s, 1.5H), 3.0 (m, 1H), 3.3 (m, 1H), 4.85 (m, 1H), 5.5 (m, 1.5H), 6.0 (d, 0.5H), 7.0 (m, 1.5H), 7.1 (m, 0.5H), 7.2 (d, 1H), 7.3 (m, 3H), 8.6 (m, 1.0H), 11.9 (d, 1H); MS m/z 456, 458.

The following compound prepared by the process of Intermediate 22 using 2,3-dichloro-N-[(1R,2R)-1-(methylamino)-2,3-dihydro-1H-inden-2-yl]-4H-thieno[3,2-b]pyrrole-5-carboxamide hydrochloride (Intermediate 16) as the amine and (2S)-5-oxotetrahydrofuran-2-carboxylic acid as the acid.
Intermediate 23: 2,3-Dichloro-N-[(1R,2R)-1-(methyl{[(2S)-5-oxotetrahydrofuran-2-yl]carbonyl}amino)-2,3-dihydro-1H-inden-2-yl]-4H-thieno[3,2-b]pyrrole-5-carboxamide

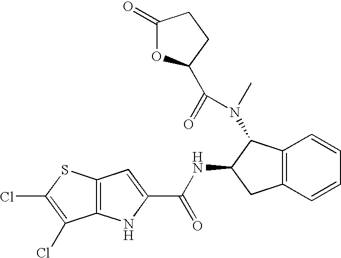

¹H NMR δ: 2.2 (m, 2H), 2.5 (m, 2H), 2.7 (s, 1.5H), 3.0 (s, 1.0H), 3.3 (m, 1H), 4.9 (m, 1H), 5.5 (m, 1.5H), 6.05 (d, 0.5H), 7.0 (m, 0.5H), 7.1 (m, 1.5H), 7.3 (m, 3H), 8.7 (m, 1H), 12.44 (d, 1.0H); MS m/z 490, 492.
Intermediate 24: 2-Chloro-N-((1R,2R)-1-{methyl[(2S)-oxiran-2-ylcarbonyl]amino}-2,3-dihydro-1H-inden-2-yl)-6H-thieno[2,3-b]pyrrole-5-carboxamide

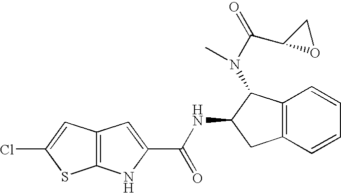

EDAC (573 mg, 3 mmol) was added to a stirred suspension of 2-chloro-N-[(1R,2R)-1-(methylamino)-2,3-dihydro-1H-inden-2-yl]-6H-thieno[2,3-b]pyrrole-5-carboxamide hydrochloride (Intermediate 1; 381.5 mg, 1.0 mmol) and potassium (2S)-oxirane-2-carboxylate (378 mg, 3 mmol) in DMF (5 mL). After stirring for 2 h at ambient temperature 20 water (20 mL) was added and the resulting solid precipitate was collected by filtration, washed with water and dried under vacuum. MS m/z 416.

The following intermediate was prepared by the method of Intermediate 24 using 2,3-dichloro-N-[(1R,2R)-1-(methylamino)-2,3-dihydro-1H-inden-2-yl]-4H-thieno[3,2-b]pyrrole-5-carboxamide hydrochloride (Intermediate 16) as the amine and potassium (2S)-oxirane-2-carboxylate as the carboxylic acid.
Intermediate 25: 2,3-Dichloro-N-((1R,2R)-1-{methyl[(2S)-oxiran-2-ylcarbonyl]amino}-2,3-dihydro-1H-inden-2-yl)-4H-thieno[3,2-b]pyrrole-5-carboxamide

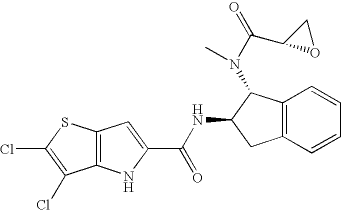

MS m/z 482.
Intermediate 26: 2-Chloro-N-((1R,2R)-1-{[(2,2-dimethyl-1,3-dioxolan-4-yl)carbonyl][2-(tetrahydro-2H-pyran-2-yloxy)ethyl]amino}-2,3-dihydro-1H-inden-2-yl)-6H-thieno[2,3-b]pyrrole-5-carboxamide

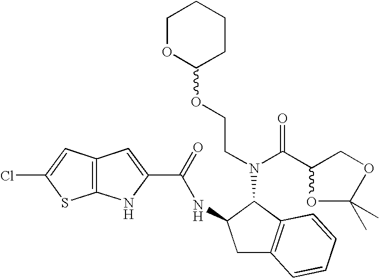

To a solution of 2-chloro-N-((1R,2R)-1-{[2-(tetrahydro-2H-pyran-2-yloxy)ethyl]amino}-2,3-dihydro-1H-inden-2-yl)-6H-thieno[2,3-b]pyrrole-5-carboxamide (230 mg, 0.5 mmol) 2,2-dimethyl-1,3-dioxolane-4-carboxylic acid potassium salt (92 mg, 0.5 mmol) in anhydrous DMA (5 ml) was added HOBT (68 mg, 0.5 mmol) and EDCI (120 mg, 0.625 mmol). The reaction was stirred at ambient temperature for approximately 24 h, water (30 ml) added and the mixture filtered and dried in vacuo to give the title compound (270 mg, 91%) as a powder.

MS m/z 586, 588 (M−H).

The following intermediates were made by the process of Intermediate 26, using 2-Chloro-N-((1R,2R)-1-{[2-(tetrahydro-2H-pyran-2-yloxy)ethyl]amino}-2,3-dihydro-1H-inden-2-yl)-6H-thieno[2,3-b]pyrrole-5-carboxamide (Intermediate 29) as the amine and (R) or (S)-2,2-dimethyl-1,3-dioxolane-4-carboxylic acid potassium salt as the carboxylate source.

Intermediate 27: 2-Chloro-N-((1R,2R)-1-{[((R)-2,2-dimethyl-1,3-dioxolan-4-yl)carbonyl][2-(tetrahydro-2H-pyran-2-yloxy)ethyl]amino}-2,3-dihydro-1H-inden-2-yl)-6H-thieno[2,3-b]pyrrole-5-carboxamide

Intermediate 28: 2-Chloro-N-((1R,2R)-1-{[((S)-2,2-dimethyl-1,3-dioxolan-4-yl)carbonyl][2-(tetrahydro-2H-pyran-2-yloxy)ethyl]amino}-2,3-dihydro-1H-inden-2-yl)-6H-thieno[2,3-b]pyrrole-5-carboxamide

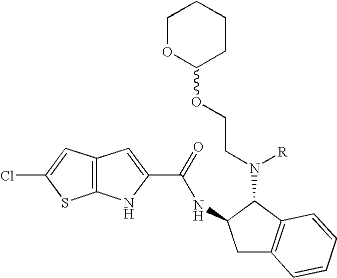

| Intermediate | | |
|---|---|---|
| No | R | MS m/z |
| 27 | 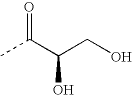 | 586, 588 |
| 28 | 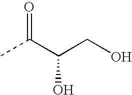 | 586, 588 |

Intermediate 29: 2-Chloro-N-((1R,2R)-1-{[2-(tetrahydro-2H-pyran-2-yloxy)ethyl]amino}-2,3-dihydro-1H-inden-2-yl)-6H-thieno[2,3-b]pyrrole-5-carboxamide

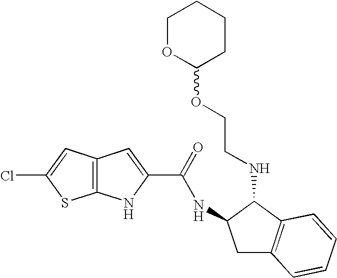

N-[(1R,2R)-1-amino-2,3-dihydro-1H-inden-2-yl]-2-chloro-6H-thieno[2,3-b]pyrrole-5-carboxamide hydrochloride (6.6 g, 20 mmol), 2-(2-iodoethoxy)tetrahydro-2H-pyran (5.12 g, 20 mmol) and DIPEA (6.85 ml, 40 mmol) in anhydrous DMA (10 ml) was heated at 60° C. for approximately 24 h. The reaction was cooled, EtOAc (150 ml) added and washed with water (6×50 ml), brine (50 ml), dried (MgSO₄) and the volatiles removed under reduced pressure. The residue was purified by flash column chromatography (EtOAc) to give the title compound (3.65 g, 40%) as a foam.

¹H NMR δ: 1.5 (m, 6H), 2.15 (m, 1H), 2.8 (m, 3H), 3.3 (m, 3H), 3.65 (m, 2H), 4.25 (d, 1H), 4.5 (m, 2H), 7.0 (s, 1H), 7.15 (s, 1H), 7.2 (m, 3H), 7.3 (m, 1H), 8.4 (d, 1H), 11.82 (m, 1H); MS m/z 458, 460 (M−H).

1. A compound of formula (1): 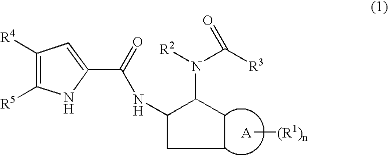
wherein:
R⁴ and R⁵ together are either —S—C(R⁶)═C(R⁷)— or —C(R⁷)═C(R⁶)—S—;
R⁶ and R⁷ are independently selected from hydrogen, halo, nitro, cyano, hydroxy, fluoromethyl, difluoromethyl, trifluoromethyl, trifluoromethoxy, carboxy, carbamoyl, (1-4C)alkyl, (2-4C)alkenyl, (2-4C)alkynyl, (1-4C)alkoxy and (1-4C)alkanoyl;
A is phenylene or heteroarylene;
n is 0, 1 or 2;
R¹ is independently selected from halo, nitro, cyano, hydroxy, carboxy, carbamoyl, N-(1-4C)alkylcarbamoyl, N,N-((1-4C)alkyl)₂carbamoyl, sulphamoyl, N-(1-4C)alkylsulphamoyl, N,N-((1-4C)alkyl)₂sulphamoyl, —S(O)_{b}(1-4C)alkyl (wherein b is 0, 1, or 2), —OS(O)₂(1-4C)alkyl, (1-4C)alkyl, (2-4C)alkenyl, (2-4C)alkynyl, (1-4C)alkoxy, (1-4C)alkanoyl, (1-4C)alkanoyloxy, hydroxy(1-4C)alkyl, fluoromethyl, difluoromethyl, trifluoromethyl, trifluoromethoxy and —NHSO₂(1-4C)alkyl;
or, when n is 2, the two R¹ groups, together with the carbon atoms of A to which they are attached, may form a 4 to 7 membered saturated ring, optionally containing 1 or 2 heteroatoms independently selected from O, S and N, and optionally being substituted by one or two methyl groups;
one of R² and R³ is selected from R_{N}a, and the other is selected from R_{N}b;
R_{N}a: (1-3C)alkyl, halo(1-3C)alkyl, dihalo(1-3)alkyl, trifluoromethyl, hydroxy(1-3C)alkyl, dihydroxy(2-3C)alkyl, cyano(1-3C)alkyl (optionally substituted on alkyl with hydroxy), methoxymethyl, ethoxymethyl, methoxyethyl, methoxymethoxymethyl, dimethoxyethyl, (hydroxy)(methoxy)ethyl, 5- and 6-membered acetals and mono- and di-methyl derivatives thereof, (amino)(hydroxy)(2-3C)alkyl, (aminocarbonyl)(hydroxy)(2-3C)alkyl, (methylaminocarbonyl)(hydroxy)(2-3C)alkyl, (dimethylaminocarbonyl)(hydroxy)(2-3C)alkyl, (methylcarbonylamino)(hydroxy)(2-3C)alkyl, (methylS(O)ₚ-)(hydroxy)(2-3C)alkyl (wherein p is 0, 1 or 2);
R_{N}b: (1-4C)alkyl, halo(1-4C)alkyl, dihalo(1-4C)alkyl, trifluoromethyl, hydroxy(1-4C)alkyl, dihydroxy(2-4C)alkyl, trihydroxy(3-4C)alkyl, cyano(1-4C)alkyl (optionally substituted on alkyl with hydroxy), (1-4C)alkoxy(1-4C)alkyl, (1-4C)alkoxy(1-4C)alkoxy(1-4C)alkyl, di[(1-4C)alkoxy](2-4C)alkyl, (hydroxy)[(1-4C)alkoxy](2-4C)alkyl, 5- and 6-membered acetals and mono- and di-methyl derivatives thereof, (amino)(hydroxy)(2-4C)alkyl, (aminocarbonyl)(hydroxy)(2-4C)alkyl, ((1-4C)alkylaminocarbonyl)(hydroxy)(2-4C)alkyl, (di(1-4C)alkylaminocarbonyl)(hydroxy)(2-4C)alkyl, ((1-4C)alkylcarbonylamino)(hydroxy)(2-4C)alkyl, ((1-4C)alkylS(O)ₚ-)(hydroxy)(2-4C)alkyl (wherein p is 0, 1 or 2);
wherein any alkyl or alkoxy group within any group in R_{N}a and R_{N}B may also optionally be substituted on an available carbon atom with a hydroxy group (provided that said carbon atom is not already substituted by a group linked by a heteroatom);
provided that if R² is (1-3C)alkyl or (1-4C)alkyl then R³ is not (1-4C)alkyl or (1-3C)alkyl;
or a pharmaceutically acceptable salt or pro-drug thereof.

2. A compound of formula (1) as claimed in claim 1, or a pharmaceutically acceptable salt or pro-drug thereof, wherein R² is selected from R_{N}a, and R³ is selected from R_{N}b, wherein R_{N}a and R_{N}b are as defined in claim 1.

3. A compound of formula (1) as claimed in claim 1, or a pharmaceutically acceptable salt or pro-drug thereof, wherein A is phenylene.

4. A compound of formula (1) as claimed in claim 1, or a pharmaceutically acceptable salt or pro-drug thereof, wherein n is 0.

5. A compound of formula (1) as claimed in claim 1, or a pharmaceutically acceptable salt or pro-drug thereof, wherein R⁶ and R⁷ are independently selected from hydrogen and halo.

6. A compound of formula (1) as claimed in claim 1, or a pharmaceutically acceptable salt or pro-drug thereof, wherein R⁶ and R⁷ are independently selected from hydrogen and chloro.

7. A compound of formula (1) as claimed in claim 1, or a pharmaceutically acceptable salt or pro-drug thereof, wherein R_{N}a is selected from (1-4C)alkyl, hydroxy(1-4C)alkyl, and (1-4C)alkoxy(1-4C)alkyl.

8. A compound of formula (1) as claimed in claim 1, or a pharmaceutically acceptable salt or pro-drug thereof, which is a compound of formula (1A): 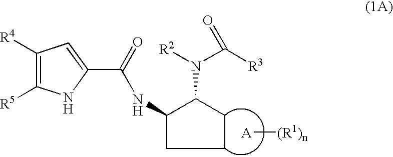
wherein R¹ to R⁷ and n are as defined in claim 1.

9. A pro-drug of a compound of formula (1) as claimed in claim 1, which pro-drug is an in-vivo hydrolysable ester.

10. A pharmaceutical composition which comprises a compound of the formula (1), or a pharmaceutically acceptable salt or in-vivo hydrolysable ester thereof, as claimed in claim 1 in association with a pharmaceutically-acceptable diluent or carrier.

11-15. (canceled)

16. A process for the preparation of a compound of formula (1) as claimed in claim 1, which process comprises: reacting an acid of the formula (2): 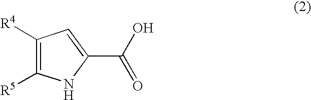
or an activated derivative thereof; with an amine of formula (3): 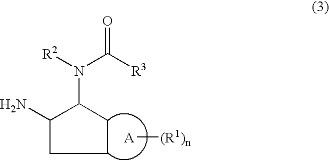
and thereafter if necessary:
i) converting a compound of the formula (1) into another compound of the formula (1);
ii) removing any protecting groups;
iii) forming a pharmaceutically acceptable salt or in vivo hydrolysable ester.

17. A compound of formula (1), or a pharmaceutically acceptable salt or pro-drug thereof, selected from:
2-chloro-N-{(1R,2R)-1-[(methoxyacetyl)(methyl)amino]-2,3-dihydro-1H-inden-2-yl}-6H-thieno[2,3-b]pyrrole-5-carboxamide;
2-chloro-N-{(1R,2R)-1-[[3-hydroxy-2-(hydroxymethyl)propanoyl](methyl)amino]-2,3-dihydro-1H-inden-2-yl}-6H-thieno[2,3-b]pyrrole-5-carboxamide;
ethyl 3-[((1R,2R)-2-{[(2-chloro-6H-thieno[2,3-b]pyrrol-5-yl)carbonyl]amino}-2,3-dihydro-1H-inden-1-yl)(methyl)amino]-3-oxopropanoate;
2-[((1R,2R)-{[(2-chloro-6H-thieno[2,3-b]pyrrol-5-yl)carbonyl]amino}-2,3-dihydro-1H-inden-1-yl)(methyl)amino]-2-oxoethyl acetate;
2-chloro-N-{(1R,2R)-1-[glycoloyl(methyl)amino]-2,3-dihydro-1H-inden-2-yl}-6H-thieno[2,3-b]pyrrole-5-carboxamide;
2-chloro-N-{(1R,2R)-1-[glyceroyl(methyl)amino]-2,3-dihydro-1H-inden-2-yl}-6H-thieno[2,3-b]pyrrole-5-carboxamide;
2-chloro-N-{(1R,2R)-1-[[(2S)-2,3-dihydroxypropanoyl](methyl)amino]-2,3-dihydro-1H-inden-2-yl}-6H-thieno[2,3-b]pyrrole-5-carboxamide;
2-chloro-N-{(1R,2R)-1-[[(2R)-2,3-dihydroxypropanoyl](methyl)amino]-2,3-dihydro-1H-inden-2-yl}-6H-thieno[2,3-b]pyrrole-5-carboxamide;
2-chloro-N-{(1R,2R)-1-[(3-hydroxypropanoyl)(methyl)amino]-2,3-dihydro-1H-inden-2-yl}6H-thieno[2,3-b]pyrrole-5-carboxamide;
2-chloro-N-{(1R,2R)-1-[glycoloyl(2-hydroxyethyl)amino]-2,3-dihydro-1H-inden-2-yl}-6H-thieno[2,3-b]pyrrole-5-carboxamide;
2-chloro-N-{(1R,2R)-1-[[(2R)-2-hydroxypropanoyl](methyl)amino]-2,3-dihydro-1H-inden-2-yl}-6H-thieno[2,3-b]pyrrole-5-carboxamide;
2-chloro-N-{(1R,2R)-1-[[(2S)-2-hydroxypropanoyl](methyl)amino]-2,3-dihydro-1H-inden-2-yl}-6H-thieno[2,3-b]pyrrole-5-carboxamide;
2,3-dichloro-N-{(1R,2R)-1-[[(2R)-2,3-dihydroxypropanoyl](methyl)amino]-2,3-dihydro-1H-inden-2-yl}-4H-thieno[3,2-b]pyrrole-5-carboxamide
2,3-dichloro-N-{(1R,2R)-1-[[(2S)-2,3-dihydroxypropanoyl](methyl)amino]-2,3-dihydro-1H-inden-2-yl}-4H-thieno[3,2-b]pyrrole-5-carboxamide;
(2S)-N¹-((1R,2R)-2-{[(2-chloro-6H-thieno[2,3-b]pyrrol-5-yl)carbonyl]amino}-2,3-dihydro-1H-inden-1-yl)-2-hydroxy-N¹-methylsuccinamide;
(2S)-N¹-((1R,2R)-2-{[(2,3-dichloro-4H-thieno[3,2-b]pyrrol-5-yl)carbonyl]amino}-2,3-dihydro-1H-inden-1-yl)-2-hydroxy-N¹-methylsuccinamide;
2,3-dichloro-N-{(1R,2R)-1-[[(2S)-2-hydroxybutanoyl](methyl)amino]-2,3-dihydro-1H-inden-2-yl}-4H-thieno[3,2-b]pyrrole-5-carboxamide;
2,3-dichloro-N-{(1R,2R)-1-[[(2S)-2-hydroxy-3-methylbutanoyl](methyl) amino]-2,3-dihydro-1H-inden-2-yl}-4H-thieno[3,2-b]pyrrole-5-carboxamide;
2,3-dichloro-N-{(1R,2R)-1-[[(2S)-4-(1,3-dioxo-1,3-dihydro-2H-isoindol-2-yl)-2-hydroxybutanoyl](methyl)amino]-2,3-dihydro-1H-inden-2-yl}-4H-thieno[3,2-b]pyrrole-5-carboxamide;
2,3-dichloro-N-{(1R,2R)-1-[[(2R)-2-hydroxy-3-(methylthio)propanoyl](methyl)amino]-2,3-dihydro-1H-inden-2-yl}4H-thieno[3,2-b]pyrrole-5-carboxamide;
tert-butyl{(2S)-3-[((1R,2R)-2-[(2,3-dichloro-4H-thieno[3,2-b]pyrrol-5-yl)carbonyl]amino)2,3-dihydro-1H-inden-1-yl)(methyl)amino]-2-hydroxy-3-oxopropyl}carbamate;
2,3-dichloro-N-{(1R,2R)-1-[[(2S)-3-cyano-2-hydroxypropanoyl](methyl)amino]-2,3-dihydro-1H-inden-2-yl}-4H-thieno[3,2-b]pyrrole-5-carboxamide;
N-{(1R,2R)-1-[(N-acetylseryl)(methyl)amino]-2,3-dihydro-1H-inden-2-yl}-2-chloro-6H-thieno[2,3-b]pyrrole-5-carboxamide;
N-{(1R,2R)-1-[(N-acetylseryl)(methyl)amino]-2,3-dihydro-1H-inden-2-yl}-2,3-dichloro-4H-thieno[3,2-b]pyrrole-5-carboxamide;
2,3-dichloro-N-{(1R,2R)-1-[methyl(L-seryl)amino]-2,3-dihydro-1H-inden-2-yl}-4H-thieno[3,2-b]pyrrole-5-carboxamide hydrochloride;
2-chloro-N-{(1R,2R)-1-[methyl(L-seryl)amino]-2,3-dihydro-1H-inden-2-yl}-6H-thieno[2,3-b]pyrrole-5-carboxamide hydrochloride;
(2S)-N¹-((1R,2R)-2-{[(2-chloro-6H-thieno[2,3-b]pyrrol-5-yl)carbonyl]amino}-2,3-dihydro-1H-inden-1-yl)-2-hydroxy-N¹-methylpentanediamide;
(2S)-N¹-((1R,2R)-2-{[(2,3-dichloro-4H-thieno[3,2-b]pyrrol-5-yl)carbonyl]amino}-2,3-dihydro-1H-inden-1-yl)-2-hydroxy-N¹-methylpentanediamide;
2-chloro-N-{(1R,2R)-1-[[(2S)-2-hydroxy-3-methoxypropanoyl](methyl) amino]-2,3-dihydro-1H-inden-2-yl}-6H-thieno[2,3-b]pyrrole-5-carboxamide;
2,3-dichloro-N-{(1R,2R)-1-[[(2S)-2-hydroxy-3-methoxypropanoyl](methyl)amino]-2,3-dihydro-1H-inden-2-yl}4H-thieno[3,2-b]pyrrole-5-carboxamide;
2,3-dichloro-N-{(1R,2R)-1-[[(2R)-2-hydroxy-3-(methylsulfonyl)propanoyl](methyl)amino]-2,3-dihydro-1H-inden-2-yl}-4H-thieno[3,2-b]pyrrole-5-carboxamide;
N-{(1R,2R)-1-[[(2S)-3-amino-2-hydroxypropanoyl](methyl)amino]-2,3-dihydro-1H-inden-2-yl}-2,3-dichloro-4H-thieno[3,2-b]pyrrole-5-carboxamide hydrochloride;
(2S)-N¹-((1R,2R)-2-{[(2,3-dichloro-4H-thieno[3,2-b]pyrrol-5-yl)carbonyl]amino}-2,3-dihydro-1H-inden-1-yl)-2-hydroxy-N¹,N⁴-dimethylsuccinamide;
(2S)-N¹-((1R,2R)-2-{[(2,3-dichloro-4H-thieno[3,2-b]pyrrol-5-yl)carbonyl]amino}-2,3-dihydro-1H-inden-1-yl)-2-hydroxy-N¹,N⁴,N⁴-trimethylsuccinamide;
2-chloro-N-{(1R,2R)-1-[glyceroyl(2-hydroxyethyl)amino]-2,3-dihydro-1H-inden-2-yl}-6H-thieno[2,3-b]pyrrole-5-carboxamide;
2-chloro-N-{(1R,2R)-1-[[(2R)-2,3-dihydroxypropanoyl](2-hydroxyethyl) amino]-2,3-dihydro-1H-inden-2-yl}-6H-thieno[2,3-b]pyrrole-5-carboxamide;
2-chloro-N-{(1R,2R)-1-[[(2S)-2,3-dihydroxypropanoyl](2-hydroxyethyl)amino]-2,3-dihydro-1H-inden-2-yl}-6H-thieno[2,3-b]pyrrole-5-carboxamide.

18. A method of producing a glycogen phosphorylase inhibitory effect in a warm-blooded animal, such as man, in need of such treatment which comprises administering to said animal an effective amount of a compound of formula (1) as claimed in claim 1.

19. A method of treating type 2 diabetes, insulin resistance, syndrome X, hyperinsulinaemia, hyperglucagonaemia, cardiac ischaemia or obesity in a warm-blooded animal, such as man, in need of such treatment which comprises administering to said animal an effective amount of a compound of formula (1) as claimed in claim 1.

20. A method of treating type 2 diabetes in a warm-blooded animal, such as man, in need of such treatment which comprises administering to said animal an effective amount of a compound of formula (1) as claimed in claim 1.
